(19) United States
(12) Patent Application Publication
(10) Pub. No.: US 2020/0345862 A1
(43) Pub. Date: Nov. 5, 2020

(54) TRANSITION METAL-BASED FUNCTIONAL MOIETIES FOR PREPARING CELL TARGETING CONJUGATES

(71) Applicant: LINXIS B.V., Amsterdam (NL)

(72) Inventors: Eugen Merkul, Amsterdam (US); Niels Jurriaan Sijbrandi, Utrecht (NL); Joey Armand Muns, Hoofddorp (NL); Augustinus Antonius Maria Silvester Van Dongen, Utrecht (NL); Paulus Johannes Gerardus Maria Steverink, Oss (NL); Hendrik Jan Houthoff, Amsterdam (NL)

(21) Appl. No.: 16/956,472

(22) PCT Filed: Dec. 19, 2018
(86) PCT No.: PCT/NL2018/050858

§ 371 Date: Jun. 19, 2020

(30) Foreign Application Priority Data
Dec. 19, 2017 (NL) 2020121

(51) Int. Cl. A61K 47/68

(52) U.S. Cl. CPC A61K 47/6855; A61K 47/6889; A61K 47/6803

(57) ABSTRACT

The disclosure relates to secondary functional moieties comprising a transition metal-based linker and a primary functional moiety bound thereto. The disclosure also relates to cell targeting conjugates comprising a linker of the invention. The disclosure further relates to a medicament comprising the cell targeting conjugate and to the use of the cell targeting conjugates in the diagnosis and treatment of cancer.

### TECHNICAL FIELD OF THE INVENTION

The present invention relates to secondary functional moieties comprising a transition metal-based linker and a primary functional moiety bound thereto. The invention also relates to cell targeting conjugates comprising a linker of the invention. The present invention further relates to a medicament comprising said cell targeting conjugate and to the use of the cell targeting conjugates in the diagnosis and treatment of cancer.

### BACKGROUND OF THE INVENTION

Cell targeting conjugates, also known as antibody-drug conjugates (ADCs), are a relatively new class of biotherapeutics that have the potency to combine the pharmacokinetics, specificity, and biodistribution of an immunoglobulin with the cell killing properties of a small-molecule drug. Delivery of drugs linked to an immunoglobulin molecule, such as an antibody, that, with preference, specifically targets a cancerous cell only, is considered a valuable tool to improve therapeutic efficacy and to reduce the systemic toxicity of drugs used for the treatment of cancer. Whereas non-targeted drug compounds typically reach their intended target cells via whole-body distribution and passive diffusion or receptor-mediated uptake over the cell membrane, targeted drugs home-in and concentrate mainly at the targeted tissues. Consequently, targeted drugs require smaller dosages while still allowing the drug to reach therapeutically effective levels inside the target cells and thus improving the therapeutic window. The targeting of drugs to specific cells is therefore a conceptually attractive method to enhance specificity, to decrease systemic toxicity, and to allow for the therapeutic use of compounds that are less suitable or unsuitable as systemic drugs.

Although the general concept of cell targeting conjugates is simple, their successful clinical use depends on many factors such as the choice of the immunoglobulin, of the cytotoxic drug and, importantly, of the method of linking the cytotoxic drug to the immunoglobulin since the pharmacokinetics, specificity, biodistribution, and toxicity of the cell targeting conjugates can be impacted by any of these building blocks. Linkers are an essential part of antibody-drug conjugates and they account for stability in circulation, pharmacokinetics, and the release of toxic drugs at the site of interest. The linker system can thus considerably affect the properties of cell targeting conjugates, and therefore it is of key importance for the efficacy and toxicity of cell targeting conjugates.

Most linking technologies make use of the covalent coupling of organic linkers to immunoglobulins via a reactive ester or a maleimide functional group, allowing the coupling to lysine or cysteine residues of the immunoglobulin, respectively. However, it is recognized that the cell targeting conjugates comprising the above mentioned covalent linker technologies are associated with e.g. a suboptimal therapeutic window. Recently, we described a pioneering approach using ethylenediamineplatinum(II) as a linker in bioconjugation reactions to develop ADCs. In a first step, ethylenediamineplatinum(II) can be coordinated to drugs bearing non-conventional functionalities such as an N-heterocyclic ligand to provide storable “semi-final products”. In a second step, a linker-drug semi-final product can be conjugated directly, specifically, and efficiently to immunoglobulins. The use of transition metal complexes has been shown to provide for a facile, elegant, and robust means to produce effective cell targeting conjugates (WO2013/103301). Based on these characteristics, transition metal based linkers, such as platinum-based linker technology, can pave the way to a modular plug-and-play ADC development platform, in which mAbs and drugs can be easily varied. The potential of said linker technology was recently demonstrated in the preparation of auristatin F-conjugated trastuzumab (trastuzumab-Lx-AF). A single dose of trastuzumab-Lx-AF outperformed its maleimide benchmark trastuzumab-mal-AF and the FDA-approved ado-trastuzumab emtansine in a xenograft mouse model of gastric cancer (NCI-N87) and of ado-trastuzumab emtansine-resistant breast cancer (JIMT-1).

Due to their unique chemical features, transition metal complexes can overcome challenges often encountered in the field of cell targeting conjugates such as the absence of chemically reactive groups for conventional conjugation chemistry or the presence of unwanted chemically reactive groups on the payload. Moreover, the aggregate formation of immunoglobulins following drug conjugation readily encountered when using classical linker systems for the generation of cell targeting conjugates can be diminished.

Additionally, the modification of the immunoglobulin, e.g. the reduction of the disulfide bridges of the hinge region of the immunoglobulin in order to liberate cysteines or the introduction of cysteines by genetic engineering, as is required in most current organic linker technologies, is not required for the present method wherein transition metal complexes are used as linkers.

Using transition metal complexes to link toxic drugs to immunoglobulins renders highly stable cell targeting conjugates having pharmacokinetic properties, specificity, and biodistribution profiles similar to the native immunoglobulin. This is particularly important because only if features such as the immunoreactivity of the cell binding moiety (e.g. an immunoglobulin) remains sufficiently high and its biodistribution profile remains unaltered, it will be possible to deliver the conjugated drug as a therapeutic compound to the place of interest in the body. Whereas cell targeting conjugates have hit the “tipping point” with the recent approvals of Adcetris® and Kadcyla®, these should be regarded as first-generation therapies in the field of cell targeting conjugates. At the current state of technology, in order to achieve a stable coupling of a drug to an antibody, ADCs need to be developed according to, often complex, stepwise conjugation routes for every particular clinical application. This approach is inefficient with respect to i.a. development time and the use of resources and has resulted in ADCs with limited applicability in terms of e.g. their balance between efficacy and toxicity (therapeutic window). The next wave of innovation in ADC development, therefore, requires cell targeting conjugates using a more versatile linker technology, the potential for greater efficacy, and a vast improvement of their therapeutic window. Hence, there is a clear need for a more rapid, efficient, and systematic development, characterization, and production of clinically relevant cell targeting conjugates.

### SUMMARY OF THE INVENTION

The current invention allows for an efficient and modular approach to ADC development and production. The invention foresees the use of primary functional moieties bound to a transition metal complex, thus forming secondary functional moieties, for ADC development. These secondary functional moieties or semi-final products can be produced easily and efficiently according to GMP, stored, and coupled to for example an unmodified antibody of interest or other applicable cell binding moieties in a facile and efficient way.

A first aspect of the present invention relates to a secondary functional moiety according to the following formula I

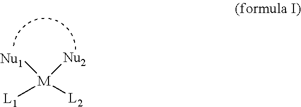

wherein M is a transition metal complex, preferably platinum (II) complex, one of the ligands L₁ or L₂ is chosen from iodide, bromide or chloride and the other ligand is a primary functional moiety; Nu is a nucleophilic group wherein Nu₁ and Nu₂ can be the same groups or different groups and which together form a bidentate ligand, under the proviso that said bidentate ligand is not ethane-1,2-diamine.

The inventors of the present secondary functional moieties have found that they are particularly useful for the preparation of cell targeting conjugates. It has further been found that for a subsequent binding of the said secondary functional moiety to a cell binding moiety (such as an antibody), thereby providing a cell targeting conjugate, it is advantageous that the second ligand is a leaving ligand preferably selected from iodide or bromide, albeit chloride may also be used but is considered less advantageous. In case chloride is used as a leaving group in the aforementioned secondary functional moiety, the chloride is preferably exchanged for bromide or iodide, preferably iodide, prior to or during the conjugation to a cell targeting moiety. It has been found that the use of iodide or bromide as a leaving ligand has a considerable and unexpected effect on the efficiency of conjugating the secondary functional moiety to the cell binding moiety and on the increased hydrolytical stability of the secondary functional moiety. Due to this increased conjugation efficiency and considering the high costs of a typical cytotoxic compound used in the ADC field, the costs of production of a cell targeting conjugate can be considerably lower.

The secondary functional moieties according to the present invention comprise a transition metal complex, such as a cis-platinum(II) complex, which complex has a primary functional moiety (e.g. an unmodified or modified cytotoxic drug) as a first ligand and iodide, bromide or chloride as a second ligand. It has been found that secondary functional moieties comprising an iodide or bromide group as a leaving ligand, in particular an iodide group as a leaving ligand, show an even improved binding efficiency to cell binding moieties (e.g. antibodies). Furthermore, the secondary functional moieties containing iodide or bromide as a leaving ligand are hydrolytically considerably more stable compared with secondary functional moieties containing chloride as a leaving ligand.

A second aspect of the present invention relates to a cell targeting conjugate comprising a reacted secondary functional moiety according to any of the previous claims, wherein the halide ligand L₁ or L₂ of the secondary functional moiety according to formula I has been displaced by a cell binding moiety.

A third aspect of the present invention relates to a pharmaceutical composition comprising a cell targeting conjugate of the invention.

### FIGURES

FIG. 1. Conjugation efficiencies depending on the leaving group of the SFM; no NaI was present in the conjugation mixture.

FIG. 2. Conjugation efficiencies depending on the leaving group of the SFM; NaI was added into the conjugation mixture.

FIG. 3. Conjugation efficiencies depending on the leaving group of the SFM; an optimal concentration of the corresponding halide salt was added into the conjugation mixture in order to stabilize the SFM.

FIG. 4. Stability of the SFM Cl-Lx-DFO(Fe) depending on the concentration of NaCl under the conjugation conditions.

FIG. 5. Stability of the SFM Br-Lx-DFO(Fe) depending on the concentration of NaBr under the conjugation conditions.

FIG. 6. Stability of the SFM I-Lx-DFO(Fe) depending on the concentration of NaI under the conjugation conditions.

### DEFINITIONS

The term “cell targeting conjugate” as used herein has its conventional meaning and refers to a primary functional moiety, such as a therapeutic compound, diagnostic compound, chelating agent, dye, or any model compound coupled to a cell binding moiety, such as an antibody, via a linker. Cell targeting conjugates involving antibodies are also referred to as antibody-drug conjugates. However, it is noted that within the realm of the present invention other types of cell binding moieties other than antibodies may be used.

The term “cell binding moiety” as used herein has its conventional meaning and refers to a member of a specific binding pair, i.e. a member of a pair of molecules wherein one of the pair of molecules has an area on its surface, or a cavity which specifically binds to, and is therefore defined as complementary with, a particular spatial and polar organization of the other molecule, so that the molecule pair has the property of binding specifically to each other. Examples of cell binding moieties according to the present invention are antibodies and antibody fragments.

The term “primary functional moiety” (PFM) as used herein refers to a molecule which has the structural ability to form a coordination bond with a transition metal complex. Typical primary functional moieties are therapeutic compounds (i.e. drugs) or diagnostic compounds (i.e. tracers or dyes) having or being equipped with a suitable coordination group which is able to make a coordinative bond to the metal center such as Pt(II).

The term “secondary functional moiety” (SFM) or “semi-final product” as used herein refers to a molecule comprising a transition metal complex, such as a platinum complex, having a first ligand and a second ligand, wherein the first ligand is a “primary functional moiety” (e.g. a modified or unmodified cytotoxic drug) as defined above, and the second ligand is iodide, bromide or chloride, preferably iodide or bromide. When allowing the secondary functional moiety to bind to a cell binding moiety, the second ligand (e.g. iodide or bromide) is substituted by the cell binding moiety. Hence, if the primary functional moiety (e.g. a modified or unmodified cytotoxic drug) and the cell binding moiety (e.g. an antibody) are bound to each other, the transition metal complex functions as a linker between them.

The term “linker” as used herein has its conventional meaning and refers to a chemical moiety which forms a bridge-like structure between a cell binding moiety and a primary functional moiety, such that the latter two are bound to each other.

The term “ligand” as used herein has its conventional meaning and refers to an ion (such as halide) or a molecule (such as a primary functional moiety) that binds to a central metal ion or atom to form a coordination complex.

The term “transition metal complex” as used herein has its conventional meaning and refers to a central transition metal atom or ion, which is called the coordination center, and a surrounding array of bound molecules or ions that are known as ligands or complexing agents. A specific example of a preferred transition metal complex used in this invention is a platinum(II) complex.

The term “Lx” as used herein refers to a structural fragment of a transition metal complex M(Nu₁-Nu₂) comprising a combination of a metal center with a bidentate ligand:

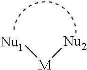

wherein M represents a metal ion or atom, which preferably is Pt(II), and Nu is a nucleophilic group wherein Nu₁ and Nu₂ can be structurally the same group or different groups and which together with the dotted line between Nu₁ and Nu₂ represent a bidentate ligand.

### DETAILED DESCRIPTION OF THE INVENTION

A first aspect of the present invention relates to a secondary functional moiety according to the following formula I

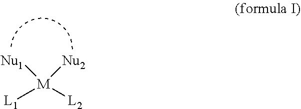

wherein M is a transition metal complex, one of the ligands L₁ or L₂ is chosen from iodide, bromide or chloride and the other ligand is a primary functional moiety; Nu is a nucleophilic group wherein Nu₁ and Nu₂ can be the same groups or different groups and which together form a bidentate ligand, under the proviso that said bidentate ligand is not ethane-1,2-diamine.

Examples of bidentate ligands as referred to in formula I are: propane-1,2-diamine (2), butane-2,3-diamine (3), 2-methylpropane-1,2-diamine (4), 2,3-diaminobutane-1,4-diol (5), 2,3-diaminopropanoic acid (6), 2,3-diaminosuccinic acid (7), 3,4-diaminobutanoic acid (8), N¹,N²-dimethylethane-1,2-diamine (9), N¹-methylethane-1,2-diamine (10), N¹,N¹-dimethylethane-1,2-diamine (11), N¹,N¹,N²-trimethylethane-1,2-diamine (12) N¹,N¹,N¹,N²,N²-tetramethylethane-1,2-diamine (13), N¹,N²-diethylethane-1,2-diamine (14), N¹,N²-dipropylethane-1,2-diamine (15), N¹,N²-diisopropylethane-1,2-diamine (16), 2-((2-aminoethyl)amino)ethan-1-ol (17), 2,2′-(ethane-1,2-diylbis(azanediyl))bis(ethan-1-ol) (18), 2,2′-(ethane-1,2-diylbis(azanediyl))bis(butan-1-ol) (19), 2,2′,2″,2′″-(ethane-1,2-diylbis(azanetriyl))tetrakis(ethan-1-ol) (20), 3-((2-aminoethyl)amino)propan-1-ol (21), (2-aminoethyl)glycine (22), 3-((2-aminoethyl)amino)propanoic acid (23), 2,2′-(ethane-1,2-diylbis(azanediyl))diacetic acid (24), 3,3′-(ethane-1,2-diylbis(azanediyl))dipropionic acid (25), 3-((2-aminoethyl)amino)propane-1-sulfonic acid (26), N¹-(2-aminoethyl)ethane-1,2-diamine (27), N¹-(2-aminoethyl)-N¹-methylethane-1,2-diamine (28), N¹,N¹-bis(2-aminoethyl)ethane-1,2-diamine (29), piperazine (30), decahydroquinoxaline (31), decahydroquinoxaline-6-carboxylic acid (32), (decahydroquinoxalin-6-yl)methanol (33), pyrrolidin-2-ylmethanamine (34), 1-(pyrrolidin-2-yl)ethan-1-amine (35), 2,2′-bipyrrolidine (36), piperidin-2-ylmethanamine (37), 1-(piperidin-2-yl)ethan-1-amine (38), 2,2′-bipiperidine (39), pyrrolidin-3-amine (40), 4-aminopyrrolidin-3-ol (41), pyrrolidin-3-ylmethanamine (42), cyclohexane-1,2-diamine (43), 4-methylcyclohexane-1,2-diamine (44), N¹,N²-dimethylcyclohexane-1,2-diamine (45), N¹,N¹,N²,N²-tetramethylcyclohexane-1,2-diamine (46), cyclohex-4-ene-1,2-diamine (47), (3R,4R,5S,6R)-3,4-diamino-6-(hydroxymethyl)tetrahydro-2H-pyran-2,5-diol (48), (4aR,6R,7R,8R,8aS)-6-methoxy-2-phenylhexahydropyrano[3,2-d][1,3]dioxine-7,8-diamine (49), cyclopentane-1,2-diamine (50), cyclobutane-1,2-diamine (51), cyclopropane-1,2-diamine (52), 1-benzylpyrrolidine-3,4-diamine (53).

Further examples of bidentate ligands as referred to in formula I are: propane-1,3-diamine (54), butane-1,3-diamine (55), butane-1,3-diamine (56), 2,4-diaminobutanoic acid (57), 2,4-diaminopentanedioic acid (58), 2,2-dimethylpropane-1,3-diamine (59), cyclobutane-1,1-diyldimethanamine (60), (tetrahydro-2H-pyran-4,4-diyl)dimethanamine (61), 2,2-bis(aminomethyl)propane-1,3-diol (62), cyclohexane-1, l-diyldimethanamine (63), 2-methylpropane-1,3-diamine (64), 1,3-diaminopropan-2-ol (65), 2-(aminomethyl)-2-methylpropane-1,3-diamine (66), 1,3-diaminopropan-2-one (67), N¹-methylpropane-1,3-diamine (68), 1,3-bis(dimethylamino)propan-2-ol (69), 1,3-bis(methylamino)propan-2-ol (70), (3-aminopropyl)glycine (71), 2-((3-aminopropyl)amino)ethan-1-ol (72), 2,2′-(propane-1,3-diylbis(azanediyl))bis(ethan-1-ol) (73), 1,4-diazepane (74), 1-amino-3-((2-hydroxyethyl)amino)propan-2-ol (75), 2,2′-((2-hydroxypropane-1,3-diyl)bis(azanediyl))bis(ethan-1-ol) (76), N¹-(3-aminopropyl)butane-1,4-diamine (77), N¹,N¹-(butane-1,4-diyl)bis(propane-1,3-diamine) (78).

Even further examples of bidentate ligands as referred to by formula I are: butane-1,4-diamine (79), 2,5-diaminopentanoic acid (80), 2-methylbutane-1,4-diamine (81), 1,4-diaminobutane-2,3-diol (82), (1,3-dioxolane-4,5-diyl)dimethanamine (83), (2-methyl-1,3-dioxolane-4,5-diyl)dimethanamine (84), (2-ethyl-1,3-dioxolane-4,5-diyl)dimethanamine (85), (2-propyl-1,3-dioxolane-4,5-diyl)dimethanamine (86), (2-isopropyl-1,3-dioxolane-4,5-diyl)dimethanamine (87), (2-phenyl-1,3-dioxolane-4,5-diyl)dimethanamine (88), (2-(2-fluorophenyl)-1,3-dioxolane-4,5-diyl)dimethanamine (89), (2-(3-fluorophenyl)-1,3-dioxolane-4,5-diyl)dimethanamine (90), (2-(4-fluorophenyl)-1,3-dioxolane-4,5-diyl)dimethanamine (91), (2-(thiophen-2-yl)-1,3-dioxolane-4,5-diyl)dimethanamine (92), (2-(furan-2-yl)-1,3-dioxolane-4,5-diyl)dimethanamine (93), cyclobutane-1,2-diyldimethanamine (94), (1s,4s)-cyclohexane-1,4-diamine (95), N¹,N^{1′}-(butane-1,4-diyl)bis(propane-1,3-diamine) (96).

A preferred bidentate ligand of a secondary functional moiety according to the present invention is represented by structures 17, 18, 21, 43, 48, 49, 54, 62, 65, 72, 73, 75, 76, 82, 87, 94 as referred to above. Even more preferred bidentate ligands of a secondary functional moiety according to the present invention are propane-1,3-diamine (54) and 1,3-diaminopropan-2-ol (65).

The inventors of the present secondary functional moieties of the invention have also found that for binding a primary functional moiety to a cell binding moiety (such as an antibody) through the linkers of the invention, it is advantageous if the second ligand L₁ or L₂ of the corresponding secondary functional moiety is iodide or bromide, preferably iodide. It has been found that the use of iodide or bromide, especially iodide, as a leaving ligand has a considerable and unexpected effect on the efficiency of conjugation of the secondary functional moiety to the cell targeting moiety and on the increased hydrolytical stability of the secondary functional moiety. Due to this increased conjugation efficiency and considering the high costs of a typical cytotoxic compound used in the ADC field, the costs of production of a cell targeting conjugate can be considerably lower.

The secondary functional moieties of the present invention having a primary functional moiety as one ligand L₁ or L₂ and iodide, bromide or chloride as the other ligand L₁ or L₂ can be conveniently prepared and stored as ready-to-use building blocks for a conjugation reaction with a cell targeting moiety or in case the leaving ligand L₁ or L₂ is iodide or bromide they can also be generated from the secondary functional moiety having chloride as a leaving ligand L₁ or L₂ in situ during the conjugation reaction with a cell targeting moiety by the addition of an iodide or a bromide releasing agent into the conjugation mixture.

In an embodiment of the present invention the platinum(II) complex of the secondary functional moiety may comprise a spacer. In such a case the primary functional moiety (e.g. an unmodified or modified cytotoxic drug) may be bound via said spacer to the platinum(II) complex rather than be bound directly to the metal center of the platinum(II) complex.

Examples of spacers are substituted or unsubstituted unbranched or branched aliphatic or heteroaliphatic chains bearing a saturated or unsaturated heterocyclic moiety, an amine or other donor group capable to bind to the metal center of the platinum(II) complex.

Furthermore, secondary functional moieties are preferably provided in an isolated form, preferably as a lyophilizate or a lyophilizate containing an excipient such as the corresponding halide salt, or they may be provided in the form of a solution, e.g. in water or water/organic solvent mixtures or in a corresponding halide salt solution. They may be stored prior to being subsequently used in a method for conjugation of a secondary functional moiety to a cell binding moiety, according to the invention.

Preferred embodiments of the secondary functional moieties according to the present invention are secondary functional moieties wherein the primary functional moiety is selected from the group consisting of a therapeutic compound, a diagnostic compound, a chelating agent, a dye or a model compound, preferably the primary functional moiety is a cytotoxic compound.

Embodiments of bidentate ligands used in secondary functional moieties of the present invention are provided above, represented by formulas 2-96 but are not restricted to. Preferred embodiments of the secondary functional moieties of the invention are secondary functional moieties wherein the therapeutic compound is a cytotoxic drug, a diagnostic compound, such as a fluorescent dye or a radiotracer ligated to a chelating compound, or a model compound.

It is particularly preferred that the cytotoxic drug is a therapeutic compound that interferes with the cytoskeleton, alkylates the DNA or intercalates into the DNA double helix, inhibits RNA polymerase II or III or inhibits a signal transduction cascade in a cellular system. Most preferably, the primary functional moiety is a cytotoxic compound. Preferred primary toxic moieties are numerous. Several examples of preferred primary functional moieties hereof are compounds chosen from the group of auristatins, dolastatins, symplostatins, maytansinoids, tubulysins, HTI-286, calicheamycins, duocarmycins, pyrrolobenzodiazepines (PBDs), indolino-benzodiazepines (IGNs), camptothecins, anthracyclines, azonafides, amanitins, cryptophycins, rhizoxins, epothilones, spliceostatins, thailanstatins, colchicines, aplyronines, taxoids, methotrexate, aminopterin, virzca alkaloids. Also preferred toxic moieties are proteinaceous toxins such as a fragment of Pseudomonas exotoxin-A, statins, ricin A, gelonin, saporin, interleukin-2, interleukin-12, viral proteins such as E4, f4, apoptin or NS1, and non-viral proteins such as HAMLET, TRAIL or mda-7.

The primary functional moiety may also be a diagnostic compound. Alternatively, the functional moiety is a fluorescent dye, such as IRDye800CW, DY-800, ALEXA FLUOR®750, ALEXA FLUOR®790, indocyanine green, FITC, BODIPY dyes such as BODIPY FL and rhodamines such as rhodamine B.

Other diagnostic compounds which may be used in the disclosure as a functional moiety are radionuclides, PET-imageable agents, SPECT-itnageable agents or MRI-imageable agents. It is also possible to couple chelating agents, such as EDTA, DPTA, and deferoxamine (Desferal® or DFO) or the macrocyclic agents DOTA or p-SCN-Bn-DOTA as a functional moiety to the metal ion complex and in a subsequent step load those chelators with therapeutic or diagnostic radionuclides such as the beta emitting agents such as ⁹⁰Y, ¹⁷⁷Th, and alpha emitters ²¹¹At or PET itosope ⁸⁹Zr and SPECT istope ^{99m}Tc, or non-radioactive metals. Alternatively, more than one kind of functional moiety can be used. In this way, it is possible to bind different functional moieties, e.g. different useful combinations of therapeutic compounds or different combinations of useful diagnostic compounds or different combinations of both, to one targeting moiety. By doing this, a preferred combination of therapeutic compounds can be delivered to the tissue of interest.

A second aspect of the present invention relates to a cell targeting conjugate comprising a secondary functional moiety as described above and in the present claims, wherein one of the ligands L₁ or L₂ of said secondary functional moiety according to formula I is a primary functional moiety and the other ligand is a cell binding moiety.

Preferred cell targeting conjugates of the invention are cell targeting conjugates wherein the bidentate ligand of the secondary functional moiety according to formula I is selected from the ligands represented by any of the formulas 2-96 as referred to above and in the claims.

Preferred embodiments of the cell targeting conjugates of the invention are cell targeting conjugates, wherein the cell binding moiety is an antibody, a single-chain antibody, an antibody fragment that specifically binds to a target cell, a monoclonal antibody, an engineered monoclonal antibody, a single-chain monoclonal antibody or monoclonal antibody that specifically binds to a target cell, a chimeric antibody, a chimeric antibody fragment that specifically binds to the target cell, and non-traditional protein scaffolds such as affibodies, anticalins, adnectins, darpins, Bicycles®, or folic acid derivatives that specifically bind to the target cells.

The cell binding moieties comprised by the cell targeting conjugates of the present invention are preferably antibodies. However, different types of antibodies may be used, such as single chain antibodies, antibody fragments that specifically bind to a target cell, monoclonal antibodies, engineered monoclonal antibodies, single chain monoclonal antibodies or monoclonal antibodies that specifically bind to a target cell, chimeric antibodies, chimeric antibody fragments that specifically bind to a target cell, and non-traditional protein scaffolds (e.g affibodies, anticalins, adnectins, darpins) that specifically bind to the target cells.

Preferably, the cell binding moiety is an antibody selected from the group of immunoglobulins targeting Her2, Her1, CD30, CD20, CD79b, CD19, EGFR, EGFRvIII or PSMA, antibodies directed against intracellular targets (such as HLA-MAGE antigen complexes) of aberrant cells (such as tumor cells).

More preferably, the cell binding moiety is an antibody selected from the group of immunoglobulins comprising trastuzumab, cetuximab, brentuximab, rituximab, ofatumumab or obinutuzumab, perferably trastuzumab.

The present invention further relates to cell targeting conjugates for the specific targeting and killing of aberrant cells, wherein the cytotoxic moiety is linked to a cell binding moiety, e.g. an antibody, via a transition metal complex, preferably a platinum(II) complex, more preferably a platinum(II) complex having a bidentate ligand represented by any of the formulas 2-96. In one embodiment, cell targeting conjugates are provided for the specific targeting and killing of aberrant cells, wherein a toxic moiety is linked to a cell binding moiety (antibody) via a transition metal complex.

In a preferred embodiment, a cell targeting conjugate according to the present invention is selected from the group consisting of: trastuzumab-Pt((1R,2R)-cyclohexane-1,2-diamine)-auristatin F, trastuzumab-Pt((1S,2S)-cyclohexane-1,2-diamine)-auristatin F, trastuzumab-Pt((1R,2S)-cyclohexane-1,2-diamine)-auristatin F, trastuzumab-Pt(N¹,N²-dimethylethane-1,2-diamine)-auristatin F, trastuzumab-Pt(propane-1,3-diamine)-auristatin F, trastuzumab-Pt(1,3-diaminopropan-2-ol)-auristatin F, trastuzumab-Pt((1R,2R)-cyclobutane-1,2-diyl)dimethanamine)-auristatin F, trastuzumab-Pt((3R,4R,5S,6R)-3,4-diamino-6-(hydroxymethyl)tetrahydro-2H-pyran-2,5-diol)-auristatin F, trastuzumab-Pt((4aR,6R,7R,8R,8aS)-6-methoxy-2-phenylhexahydropyrano[3,2-d][1,3]dioxine-7,8-diamine)-auristatin F, trastuzumab-Pt(2-((2-aminoethyl)amino)ethan-1-ol)-auristatin F, trastuzumab-Pt(2,2′-(ethane-1,2-diylbis(azanediyl))bis(ethan-1-ol))-auristatin F.

In another preferred embodiment, the cell targeting conjugates according to the present invention are selected from the group comprising anti-EGFRvIII antibody-Pt(1,3-diaminopropan-2-ol)-PNU-159682, anti-MAGE-HLA peptide complex antibody-Pt(1,3-diaminopropan-2-ol)-alfa-amanitin, MAGE-HLA peptide complex antibody-Pt(1,3-diaminopropan-2-ol)-PBD, and brentuximab-Pt(1,3-diaminopropan-2-ol)-alfa-amanitin.

In a particular preferred embodiment, the cell targeting conjugate comprises as the transition metal complex a platinum (II) complex, as a cell binding moiety trastuzumab and as the primary functional moiety an auristatin (such as auristatin F, auristatin E, monomethyl auristatin F or monomethyl auristatin E); preferably, auristatin F is used.

A further aspect of the present invention relates to a cell targeting conjugate as described above for use in the treatment of cancer in mammals, in particular humans.

Preferably, the cell targeting conjugate for use in the treatment of cancer according to the invention is for use in the treatment of colorectal cancer, breast cancer, pancreatic cancer, and non-small cell lung carcinomas.

In a further embodiment, the cell targeting conjugate for use in the treatment of cancer according to the invention is for use in the treatment of breast cancer, wherein said breast cancer has a low expression level of Her2.

The present invention further relates to a composition comprising cell targeting conjugates of the invention further comprising a radionuclide such as ^{195m}Pt in the secondary functional moiety. The use of ^{195m}Pt allows the characterization and validation of Lx-based cell targeting conjugates in vivo by using a dual-labeling approach combining ^{195m}Pt counting and ⁸⁹Zr-immuno-PET imaging. The combined use of ⁸⁹Zr and ^{195m}Pt provides the capability of sensitive and direct detection of the Lx linker apart from the antibody and the primary functional moiety, i.a. a drug or a diagnostic agent. The dual labeling strategy can thus demonstrate the in vivo stability of cell targeting conjugates, the in vivo uptake and retention of cell targeting conjugates in tumors and normal organs as a function of the DAR, and the sequestration of the platinum-based linker (Lx) in the body.

The present invention will now be elucidated further by means of the following non-limiting examples.

### EXAMPLES

### Example 1: Example of LxCl₂ Complex Used for the Synthesis of Cl-Lx-PFM Complexes (Chlorido Lx-“Semi-Final Products”)

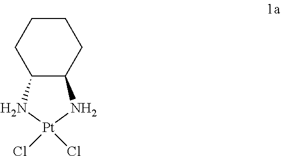

Compound 1a was purchased from Sigma-Aldrich, product code 404322, [52691-24-4].

### Example 2: Example of LxBr₂ Complex Used for the Synthesis of Br-Lx-PFM Complexes (Bromido Lx-“Semi-Final Products”)

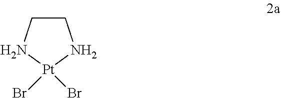

### 2.1. Synthesis and Analytical Characterization of PtBr₂(Ethane-1,2-Diamine) (2a)

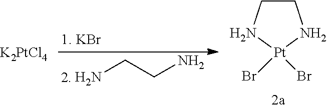

KBr (2.38 g, 20 mmol) was added to a solution of K₂PtCl₄ (415 mg, 1.0 mmol) in water (25 mL). The mixture was stirred at room temperature for 24 h, then the resulting brown mixture was filtered, ethane-1,2-diamine (81 μL, 1.2 mmol) was added to the filtrate, and the mixture was stirred at room temperature for 18 h. The precipitate was collected by filtration, thoroughly washed with water, and dried first under suction on the filter for 1 h. Then, the filter cake (335 mg of a yellow solid) was transferred into a flask and slurry-washed in MeOH (5 mL) for 1 h, collected by filtration, the filter cake was washed with MeOH, and then dried under reduced pressure for 12 h to obtain a yellow solid (298 mg, 72% yield).

Elemental analysis calc for C₂H₈Br₂N₂Pt: C, 5.79; H, 1.94; N, 6.75; found: C, 5.90; H, 1.87; N, 6.63. ¹⁹⁵Pt-NMR (86 MHz, DMF-d₇): δ −2628.

### Example 3: Examples of LxI₂ Complexes Used for the Synthesis of I-Lx-PFM Complexes (Iodido Lx-“Semi-Final Products”)

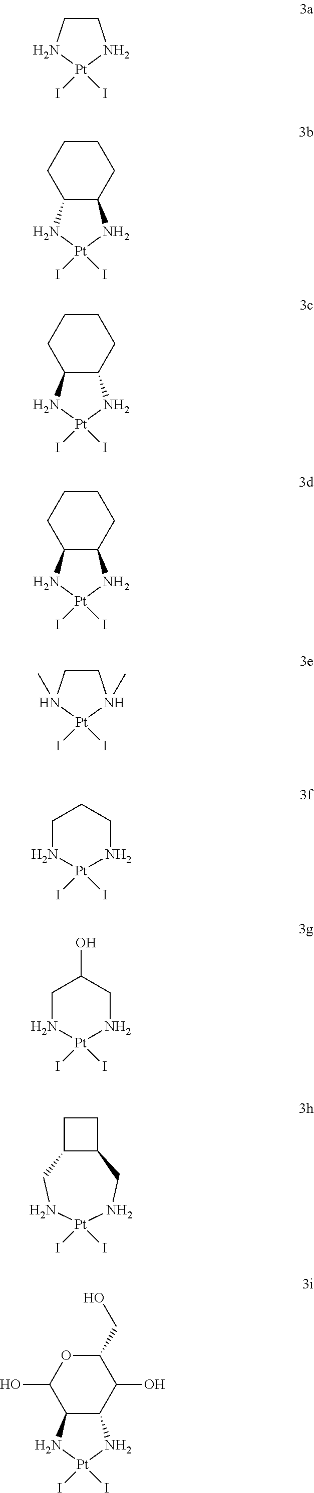

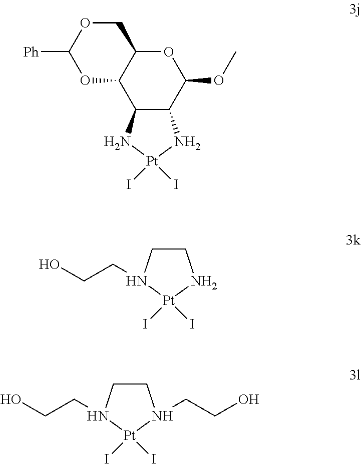

### 3.1. General Synthesis of Complexes PtI₂(Bidentate Ligand) 3a-h and 3j-1 (Exemplified for the Complex 3a) and Analytical Data of the Complex Pt(Ethane-1,2-Diamine)I₂ (3a)

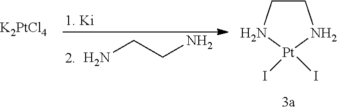

KI (33.2 g, 0.2 mol) was added to a solution of K₂PtCl₄ (4.15 g, 10 mmol) in water (200 mL). The mixture was stirred at room temperature for 22 h, then the resulting dark mixture was filtered, ethane-1,2-diamine (800 μL, 12 mmol) was added to the filtrate, and the mixture was stirred at room temperature for 23 h. A yellow precipitate started to form immediately upon addition of ethane-1,2-diamine. The precipitate was collected by filtration, thoroughly washed with water, and dried first under suction on the filter for 3-4 h and then under reduced pressure for 12 h to obtain a yellow solid (4.85 g, 95% yield).

Elemental analysis calc for C₂H₈I₂N₂Pt: C, 4.72; H, 1.58; N, 5.50; found: C, 4.68; H, 1.44; N, 5.30. ¹⁹⁵Pt-NMR (86 MHz, DMF-d₇): δ −3450. Lit (Inorg. Chem. 1992, 31, p. 5447): −3450.

HPLC (Grace Alltima C18, 25×4.6 mm, 5 μm) indicated that the product was 100% pure (retention time 9.8 min; gradient: 5 to 50% MeCN/0.1% TFA in water/0.1% TFA in 18 min measured at a wavelength of 273 nm).

Following complexes Pt(bidentate ligand)I₂ 3 were obtained in a similar way:

TABLE 1
Obtained complexes Pt(bidentate ligand)I₂ 3

| Com- | | | | Color of |
|---|---|---|---|---|
| plex | Amount of | Amount of bidentate | | obtained |
| 3 | K₂PtCl₄ | ligand | Isolated yield | solid |
| 3b | 830 mg (2.0 mmol) | 280 mg (2.4 mmol) | 1.09 g, 97% | Yellow |
| 3c | 830 mg (2.0 mmol) | 280 mg (2.4 mmol) | 1.08 g, 96% | Yellow |
| 3d | 830 mg (2.0 mmol) | 294 μL (2.4 mmol) | 1.07 g, 95% | Yellow |
| 3e | 830 mg (2.0 mmol) | 261 μL (2.4 mmol) | 1.04 g, 97% | Yellow |
| 3f | 830 mg (2.0 mmol) | 202 μL (2.4 mmol) | 986 mg, 94% | Yellow |
| 3g | 415 mg (1.0 mmol) | 223 mg (2.4 mmol) | 404 mg, 75% | Yellow |
| 3h | 830 mg (2.0 mmol) | 248 μL (2.0 mmol) | 1.03 g, 91% | Beige- |
| | | | | yellow |
| 3j | 74 mg (0.18 mmol) | 50 mg (0.18 mmol)¹ | 123 mg, 95% | Orange |
| 3k | 830 mg (2.0 mmol) | 252 mg (2.4 mmo1)² | 1.02 g, 92% | Yellow |
| 3l | 830 mg (2.0 mmol) | 367 mg (2.4 mmol) | 960 mg, 80% | Yellow- |
| | | | | orange |

¹dissolved in MeOH before addition
²dissolved in water before addition

### 3.1.1. Analytical Data of the Complex Pt((1R,2R)-cyclohexane-1,2-diamine)I₂ (3b)

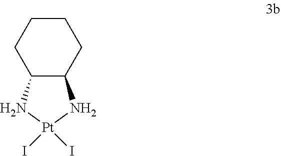

Elemental analysis calc for C₆H₁₄I₂N₂Pt: C, 12.80; H, 2.51; N, 4.98; found: C, 12.77; H, 2.42; N, 4.79. ¹⁹⁵Pt-NMR (86 MHz, DMF-d₇): δ −3421.

### 3.1.2. Analytical Data of the Complex Pt((1S,2S)-cyclohexane-1,2-diamine)I₂ (3c)

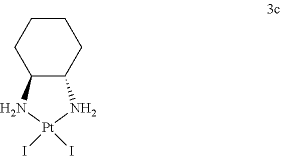

Elemental analysis calc for C₆H₁₄I₂N₂Pt: C, 12.80; H, 2.51; N, 4.98; found: C, 12.71; H, 2.35; N, 4.85.

### 3.1.3. Analytical Data of the Complex Pt((1R,2S)-cyclohexane-1,2-diamine)I₂ (3d)

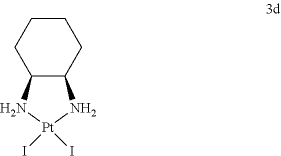

Elemental analysis calc for C₆H₁₄I₂N₂Pt: C, 12.80; H, 2.51; N, 4.98; found: C, 12.90; H, 2.36; N, 4.78. ¹⁹⁵Pt-NMR (86 MHz, DMF-d₇): δ −3399.

### 3.1.4. Analytical Data of the Complex Pt(N¹,N²-dimethylethane-1,2-diamine)I₂ (3e)

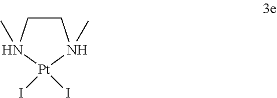

Elemental analysis calc for C₄H₁₂I₂N₂Pt: C, 8.95; H, 2.25; N, 5.22; found: C, 8.83; H, 2.08; N, 5.06. ¹⁹⁵Pt-NMR (86 MHz, DMF-d₇): δ −3431.

### 3.1.5. Analytical Data of the Complex PtI₂(propane-1,3-diamine) (3f)

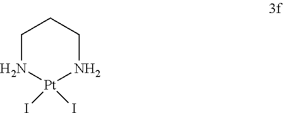

After isolation and initial drying step, the material was additionally slurry-washed in MeOH, filtered, washed with MeOH, and dried.

Elemental analysis calc for C₃H₁₀I₂N₂Pt: C, 6.89; H, 1.93; N, 5.36; found: C, 6.91; H, 1.85; N, 5.13. ¹⁹⁵Pt-NMR (86 MHz, DMF-d₇): δ −3330.

HPLC (Grace Alltima C18, 25×4.6 mm, 5 μm) indicated that the product was 100% pure (retention time 13.6 min; gradient: 5 to 50% MeCN/0.1% TFA in water/0.1% TFA in 18 min measured at a wavelength of 223 nm).

### 3.1.6. Analytical Data of the Complex Pt(1,3-diaminopropan-2-ol)I₂ (3g)

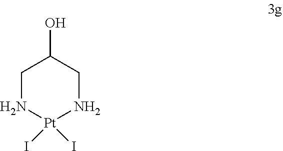

After isolation and initial drying step, the material was additionally slurry-washed in MeOH, filtered, washed with MeOH, and dried.

Elemental analysis calc for C₃H₁₀I₂N₂OPt: C, 6.68; H, 1.87; N, 5.20; found: C, 6.76; H, 1.78; N, 4.91. ¹⁹⁵Pt-NMR (86 MHz, DMF-d₇): δ −3354.

HPLC (Grace Alltima C18, 25×4.6 mm, 5 μm) indicated that the product was 100% pure (retention time 12.1 min; gradient: 5 to 50% MeCN/0.1% TFA in water/0.1% TFA in 18 min measured at a wavelength of 273 nm).

### 3.1.7. Analytical Data of the Complex Pt((1R,2R)-cyclobutane-1,2-diyl)dimethanamine)I₂ (3h)

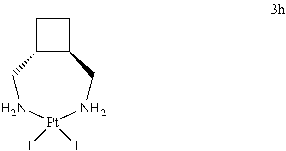

After isolation and initial drying step, the material was additionally slurry-washed in MeOH, filtered, washed with MeOH, and dried.

Elemental analysis calc for C₆H₁₄I₂N₂Pt: C, 12.80; H, 2.51; N, 4.98; found: C, 12.99; H, 2.43; N, 4.68. ¹⁹⁵Pt-NMR (86 MHz, DMF-d₇): δ −3325.

### 3.1.8. Analytical Data of the Complex Pt((4aR,6R,7R,8R,8aS)-6-methoxy-2-phenylhexahydropyrano[3,2-d][1,3]dioxine-7,8-diamine)I₂ (3j)

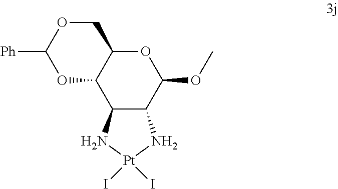

Elemental analysis calc for C₁₄H₂₀I₂N₂O₄Pt: C, 23.06; H, 2.76; N, 3.84; found: C, 23.09; H, 2.65; N, 3.73. ¹⁹⁵Pt-NMR (86 MHz, DMF-d₇): δ −3434.

### 3.1.9. Analytical Data of the Complex Pt(2-((2-aminoethyl)amino)ethan-1-ol)I₂ (3k)

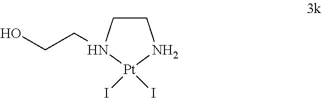

Elemental analysis calc for C₄H₁₂I₂N₂OPt: C, 8.69; H, 2.19; N, 5.07; found: C, 8.69; H, 2.06; N, 4.88. ¹⁹⁵Pt-NMR (86 MHz, DMF-d₇): δ −3438.

HPLC (Grace Alltima C18, 25×4.6 mm, 5 μm) indicated that the product was 100% pure (retention time 11.2 min; gradient: 5 to 50% MeCN/0.1% TFA in water/0.1% TFA in 18 min measured at a wavelength of 273 nm).

### 3.1.10. Analytical Data of the Complex Pt(2,2′-(ethane-1,2-diylbis(azanediyl))bis(ethan-1-ol))I₂ (3l)

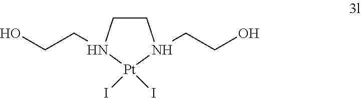

Elemental analysis calc for C₆H₁₆I₂N₂O₂Pt: C, 12.07; H, 2.70; N, 4.69; found: C, 12.03; H, 2.58; N, 4.44. ¹⁹⁵Pt-NMR (86 MHz, DMF-d₇): δ −3443.

### 32. Synthesis of the Complex Pt((3R,4R,5S,6R)-3,4-diamino-6-(hydroxymethyl)tetrahydro-2H-pyran-2,5-diol)I₂ (3i)

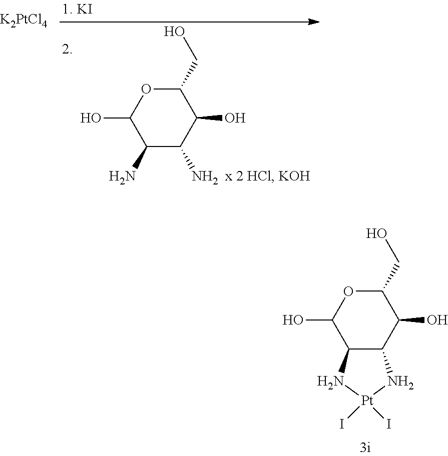

Prepared according to Berger et al., ChemMedChem 2007, 2, 505-514.

KI (531 mg, 3.2 mmol) was added to a solution of K₂PtCl₄ (266 mg, 0.64 mmol) in water (1.3 mL). The mixture was stirred at room temperature for 30 min, then the resulting dark mixture was filtered, and a solution of (3R,4R,5S,6R)-3,4-diamino-6-(hydroxymethyl)tetrahydro-2H-pyran-2,5-diol dihydrochloride (250 mg, 1.0 mmol) and KOH (98 mg, 1.5 mmol) in water (400 μL) filtered through a pad of Celite, was added to the filtrate. The mixture was stirred at room temperature for 22 h. A precipitate started to form immediately upon addition of the solution of (3R,4R,5S,6R)-3,4-diamino-6-(hydroxymethyl)tetrahydro-2H-pyran-2,5-diol. The precipitate was collected by filtration, washed with cold water (1.5 mL), followed by cold acetone (1 mL), and dried first under suction on the filter for 1 h and then under reduced pressure for 12 h to obtain a dark brown solid (162 mg, 43% yield).

¹⁹⁵Pt-NMR (86 MHz, DMF-d₇): δ −3423, −3430 (mixture of epimers).

### Example 4: Examples of Chlorido Lx-“Semi-Final Products” Cl-Lx-PFM (Chlorido SFMs)

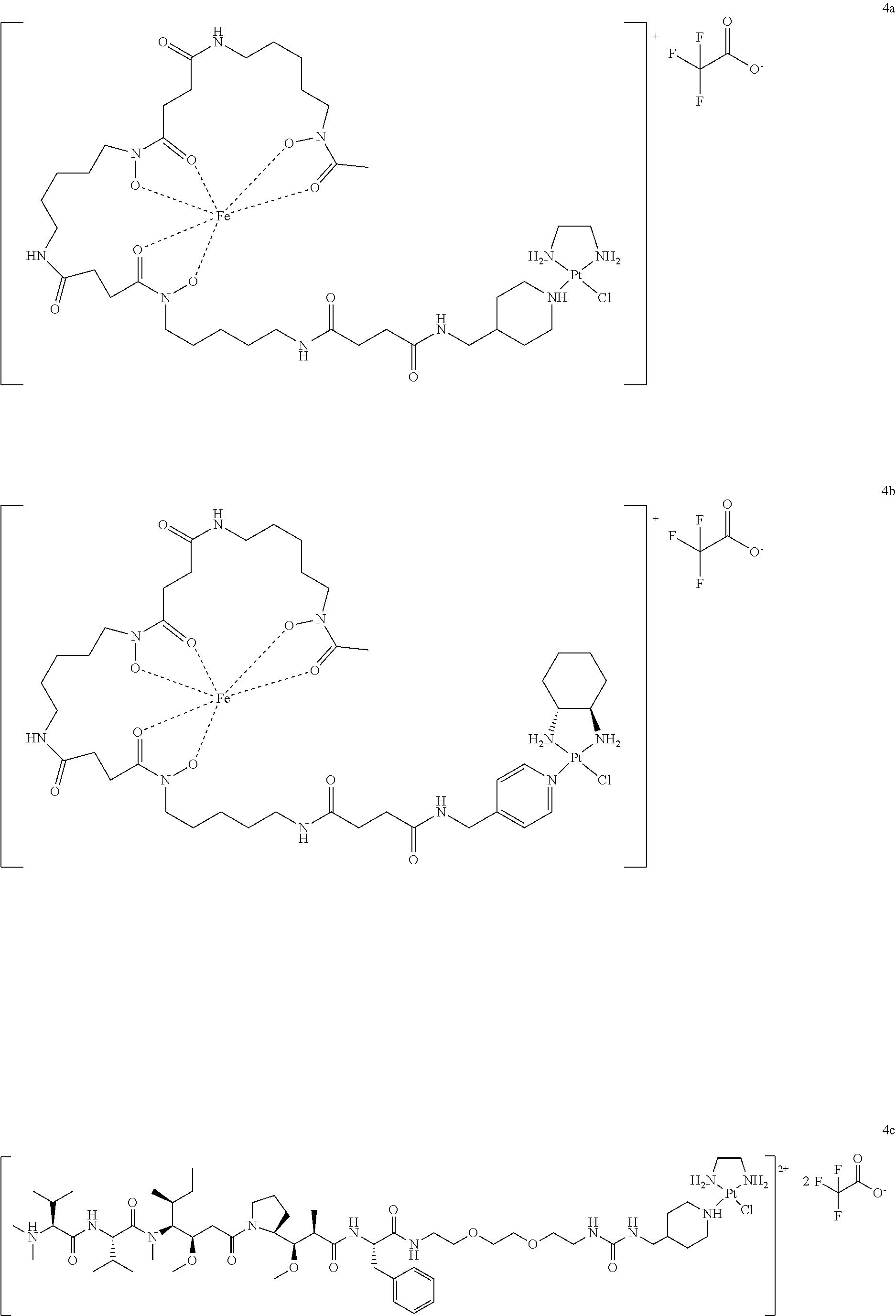

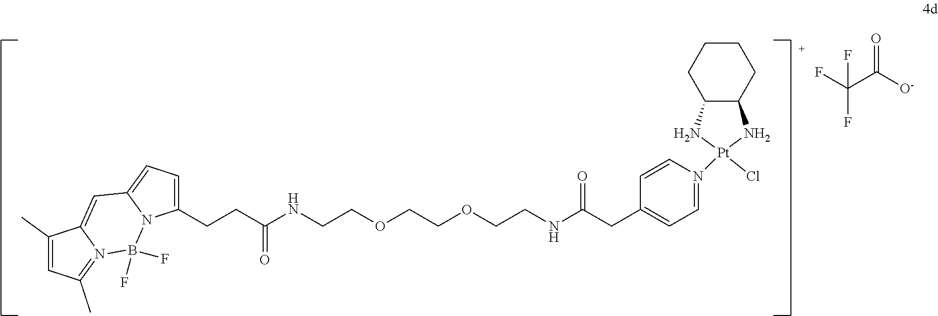

### 4.1. Synthesis and Analytical Characterization of [PtCl((Fe)DFO-pip)(ethane-1,2-diamine)]⁺ TFA⁻ (4a) is Described in Sijbrandi et al., Cancer Res. 2017, 72, 257-267

### 4.2. Synthesis and Analytical Characterization of [PtCl((Fe)DFO-suc-py)((1R,2R)-(−)-1,2-diaminocyclohexane)]⁺ TFA⁻ (4b)

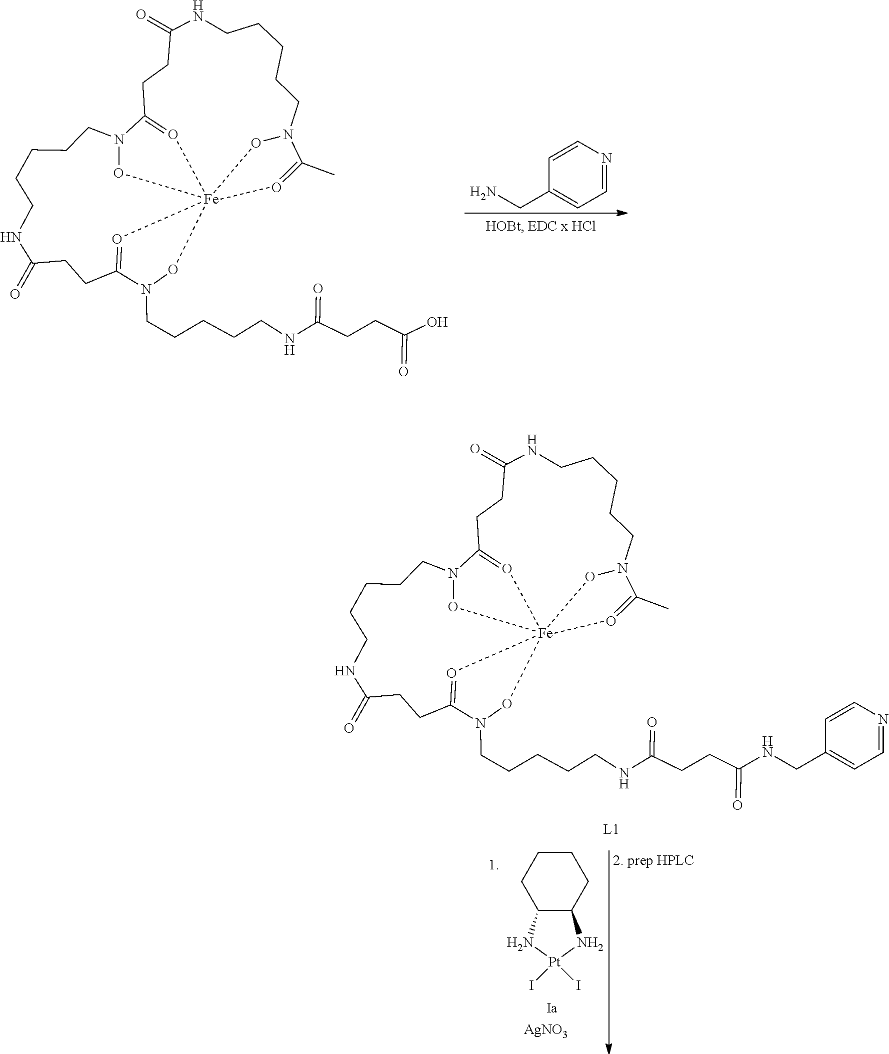

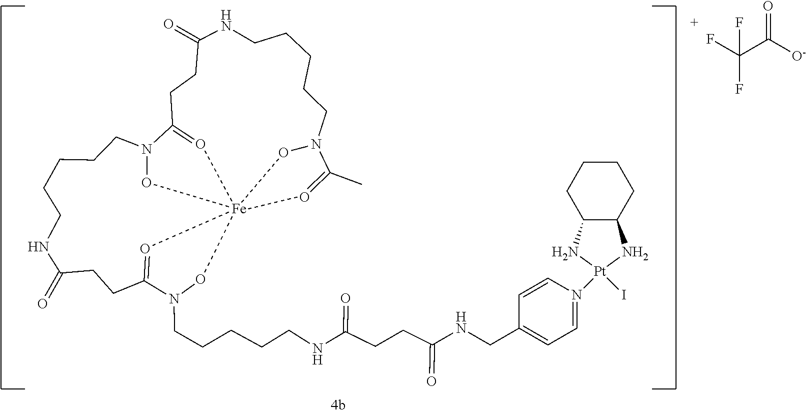

### 4.2.1. Synthesis of the Ligand (Fe)DFO-suc-py (L1)

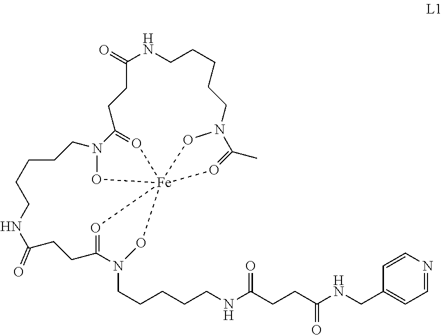

Prepared according to Verel et al., J. Nucl. Med. 2003, 44, 1271-1281.

N-Succinyl Desferal-Fe(III) ((Fe)DFO-suc; 89 mg, 124 μmol) was dissolved in DMF (1.2 mL) and HOBt (25.2 mg, 186 μmol), EDC×HCl (35.7 mg, 186 μmol), DIPEA (43 μL, 248 μmol) and pyridin-4-ylmethanamine (14 μL, 137 μmol) were sequentially added. The mixture was stirred for 20 h, concentrated, and the residue was dissolved in water and purified by Sep-Pak C18 Plus columns. The product was eluted from the columns and lyophilized resulting in a dark red solid (124 mg, 83% yield).

HRMS (ESI⁺) C₃₅H₅₆FeN₈O₁₀ [M+H]⁺ calc 804.3463, found 804.3516.

### 4.2.2. Synthesis of the Complex [PtCl((Fe)DFO-suc-py)((1R,2R)-(−)-1,2-diaminocyclohexane)]⁺ TFA⁻ (4b)

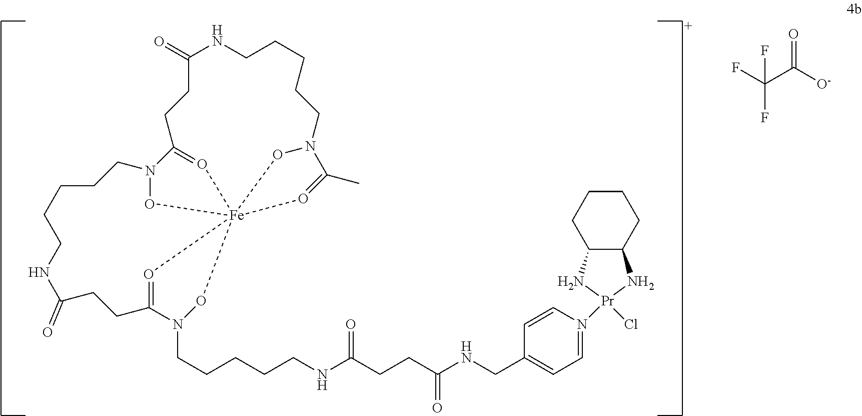

AgNO₃ (41 mg, 0.241 mmol) was added to a suspension of PtCl₂((1R,2R)-(−)-1,2-diaminocyclohexane) (1a) (87 mg, 0.229 mmol) in DMF (1 mL). After stirring for 24 h, the grey precipitate was filtered through Celite, which was then rinsed with DMF (2×0.5 mL). Then, 357 μL of this solution (1.1 eq. of activated Pt-complex) were added to (Fe)DFO-suc-py (L1) (30 mg, 0.037 mmol). The mixture was stirred for 24 h under argon after which HPLC indicated full conversion. The solvent was evaporated under reduced pressure, after which the residue was dissolved in a mixture of water and methanol. Purification was performed by preparative reverse-phase HPLC (Grace Alltima C18 5 μm column, 22×250 mm; gradient: 15 to 25% MeCN/0.1% TFA in water/0.1% TFA in 36 min). Product fractions were collected on ice and immediately frozen and lyophilized resulting in a dark red solid (10 mg, 21% yield).

HRMS (ESI⁺) C₄₁H₆₉³⁵ClFeN₁₀O₁₀¹⁹⁵Pt [M]⁺ calc 1147.3885, found 1147.3672; C₄₁H₆₉³⁵ClFeN₁₀NaO₁₀¹⁹⁵Pt [M+Na]²⁺ calc 585.1891, found 585.1771.

HPLC (Grace Alltima C18 5 column, 25×4.6 mm) indicated that the product was 97.2% pure (retention time 14.2 min; gradient: 5 to 50% MeCN/0.1% TFA in water/0.1% TFA in 18 min measured at a wavelength of 430 nm).

### 4.3. Synthesis and Analytical Characterization of Pt(auristatin F-(4-(12-amino-3-oxo-7,10-dioxa-2,4-diazadodecyl)piperidine))Cl(ethane-1,2-diamine) (4c) is Described in Sijbrandi et at, Cancer Res. 2017, 72, 257-267

### 4.4. Synthesis and Analytical Characterization of [BODIPY FL-PEG₂-py-PtCl((1R,2R)-(−)-1,2-diaminocyclohexane)]⁺ TFA⁻ (4d)

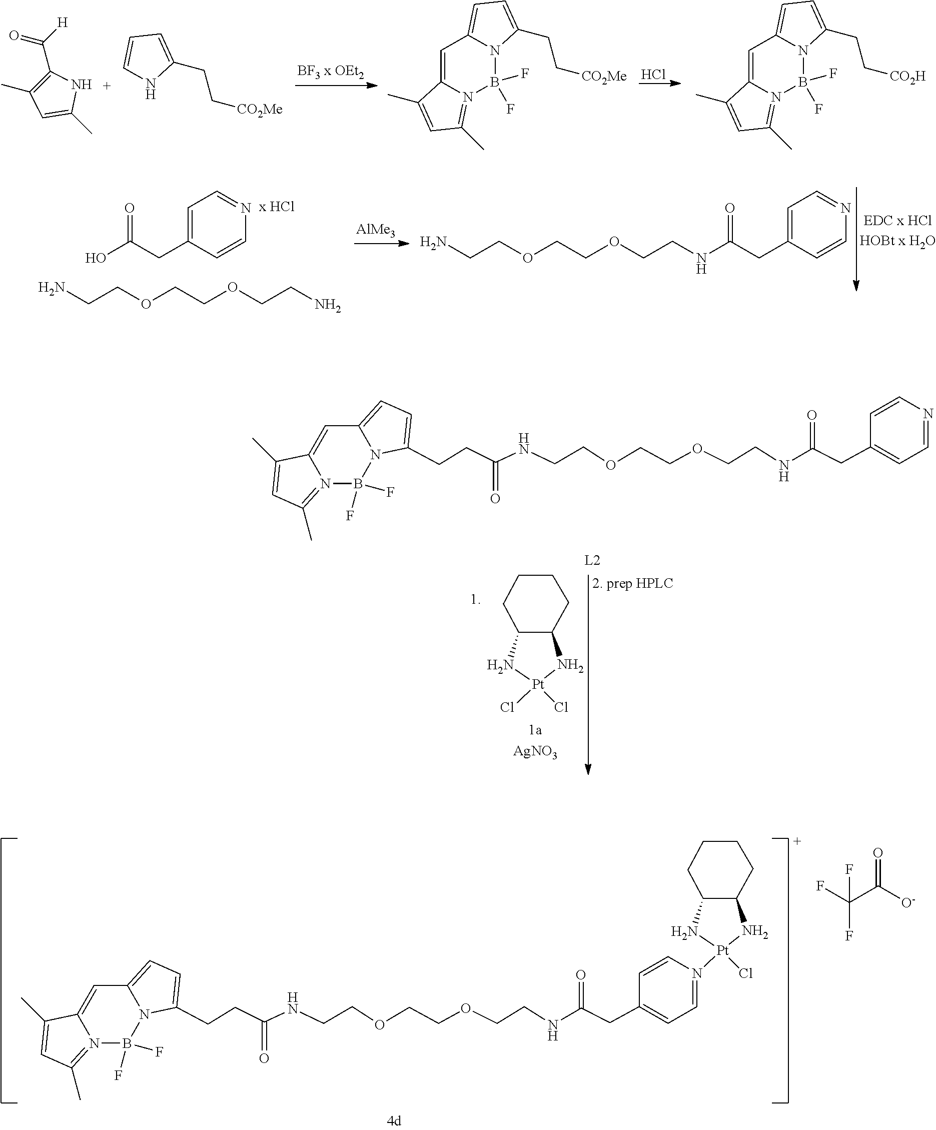

### 4.4.1. Synthesis of BODIPY FL Methyl Ester

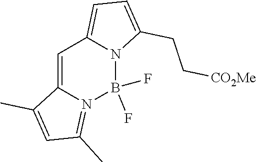

Prepared according to Gießler et al., Eur. J. Org. Chem 2010, 3611-3620.

Methyl 3-(1H-pyrrol-2-yl)propanoate (780 mg, 4.84 mmol, 1.0 eq.) and 3,5-dimethyl-1H-pyrrole-2-carbaldehyde (690 mg, 5.32 mmol, 1.1 eq.) were dissolved in DCM (50 mL) and cooled to 0° C. To this mixture, a solution of POCl₃ (500 μL, 5.36 mmol, 1.1 eq.) in DCM (5 mL) was added dropwise. The reaction mixture was stirred for 30 min at 0° C. and for 6 h at room temperature. The resulting black solution was again cooled to 0° C. and treated with BF₃×OEt₂ (2.4 mL, 19.5 mmol, 4.0 eq.) and DIPEA (3.5 mL, 20.1 mmol, 4.2 eq.) and stirred for 12 h with gradual warming to room temperature. Then, the mixture was cooled to 0° C. and water (100 mL) was added. The mixture was filtered through Celite which was rinsed with DCM (4×25 mL), the filtrate phases were separated and the aqueous layer was extracted with DCM (3×50 mL). The combined organic layers were dried with sodium sulfate and the solvents were removed under reduced pressure. The residue was absorbed on Celite and purified by column chromatography (eluent: 10-0% petroleum ether/DCM) to afford a red solid (1.00 g, 68% yield).

¹H NMR (400 MHz, CDCl₃): δ 7.08 (s, 1H), 6.88 (d, J=3.4 Hz, 1H), 6.26 (d, J=3.6 Hz, 1H), 6.11 (s, 1H), 3.69 (s, 3H), 3.29 (t, J=7.6 Hz, 2H), 2.77 (t, J=7.6 Hz, 2H), 2.56 (s, 3H), 2.25 (s, 3H).

### 4.4.2. Synthesis of BODIPY FL

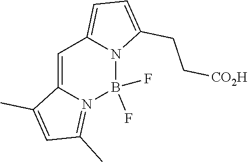

Prepared according to Gießler et al., Eur. J. Org. Chem 2010, 3611-3620.

The BODIPY methyl ester (494 mg, 1.61 mmol) was dissolved in THE (75 mL) and 4.5 M HCl (75 mL). This mixture was stirred for 47 h at room temperature. Subsequently, DCM (300 mL) was added and the phases were separated. The aqueous layer was extracted with DCM (100 mL), the combined organic layers were dried with sodium sulfate and the solvents were removed under reduced pressure. The residue was purified by column chromatography (eluent: 0-0.5% MeOH/DCM+0.1% AcOH), followed by precipitation with n-pentane to afford a red solid (276 mg, 59% yield).

¹H NMR (400 MHz, CDCl₃): δ 10.1 (br s, 1H), 7.09 (s, 1H), 6.88 (d, J=3.4 Hz, 1H), 6.29 (d, J=3.6 Hz, 1H), 6.12 (s, 1H), 3.30 (t, J=7.6 Hz, 2H), 2.83 (t, J=7.6 Hz, 2H), 2.57 (s, 3H), 2.25 (s, 3H).

### 4.4.3. Synthesis of N-(2-(2-(2-aminoethoxy)ethoxy)ethyl)-2-(pyridin-4-yl)acetamide (PEG₂-py Spacer)

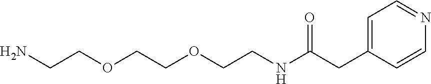 [SMILES: NCCOCCOCCNC(=O)Cc1ccncc1]

2-(Pyridin-4-yl)acetic acid hydrochloride (183 mg, 1.0 mmol, 1.0 eq.) and 2,2′-(ethane-1,2-diylbis(oxy))diethanamine (747 μL, 5.0 mmol, 5.0 eq.) were dissolved in dry and degassed toluene (5 mL). Subsequently, a 2 M solution of AlMe₃ in toluene (0.5 mL, 1.0 mmol, 1.0 eq.) was added and the resulting reaction mixture was stirred for 1 h at 90° C. The reaction mixture was then allowed to cool to room temperature over the course of 1 h and was cooled further to 0° C., followed by the addition of isopropanol (1 mL) and a 7 M solution of NH₃ in MeOH (0.14 mL), and warmed to room temperature. The yellow mixture was filtered and the solvents were removed under reduced pressure to give a green oil. This oil was dissolved in DCM and the formed precipitate was again removed by filtration. The solvent was removed under reduced pressure, after which the residue was purified by column chromatography (eluent: DCM/MeOH/NH_{3aq.} 100:9:1 to 100:9:1.5) to afford a pale yellow oil (129 mg, 48% yield).

HRMS (ESI⁺) C₁₃H₂₂N₃O₃ [M+H]⁺ calc 268.1656, found 268.1645.

¹H NMR (400 MHz, CDCl₃): δ 8.55-8.52 (m, 2H), 7.25-7.22 (m, 2H), 6.67 (s, 1H), 3.59-3.56 (m, 4H), 3.55-3.47 (m, 6H), 3.47-3.42 (m, 2H), 2.88-2.83 (m, 2H), 1.76 (s, 2H).

HPLC (Grace Alltima C18 5 μm column, 25×4.6 mm) indicated that the product was 100% pure (retention time 15.2 min; gradient: 20 to 100% MeCN/0.1% TFA in water/0.1% TFA in 20 min measured at a wavelength of 210 nm).

### 4.4.4. Synthesis of BODIPY FL-PEG₂-py Ligand (L2)

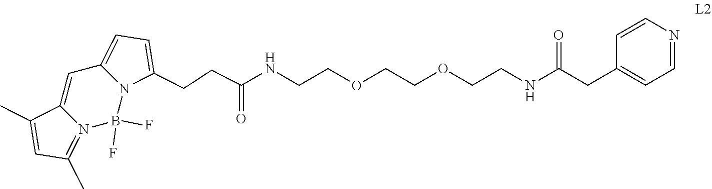

BODIPY FL (33 mg, 112 μmol, 1.0 eq.), EDC×HCl (24 mg, 123 μmol, 1.1 eq.), and HOBt hydrate (19 mg, 123 μmol, 1.1 eq.) where dissolved in DCM (1 mL) and stirred for 5 min. To this mixture PEG₂-py spacer (30 mg, 112 μmol, 1.0 eq.) was added, followed by DIPEA (41.0 μL, 236 μmol, 2.1 eq.), and the mixture was stirred for 18 h at room temperature. Subsequently, the mixture was diluted with DCM (25 mL) and washed with 0.14 M NaOH (32 mL). The two phases were separated, the aqueous layer was extracted with DCM (5×5 mL), and the combined organic layers were dried with sodium sulfate. The solvent was removed under reduced pressure and the residue was purified by column chromatography (eluent: 1-5.5% MeOH in DCM) to obtain a red oil (30 mg, 49% yield).

HRMS (ESI⁺) C₂₇H₃₅BF₂N₅O₄ [M+H]⁺ calc 542.2745, found 542.2755.

¹H NMR (250 MHz, CDCl₃): δ 8.5 (br s, 2H), 7.23-7.18 (m, 2H), 7.06 (s, 1H), 6.89-6.85 (m, 1H), 6.49-6.40 (m, 1H), 6.30-6.26 (m, 2H), 6.11 (s, 1H), 3.54-3.36 (m, 14H), 3.27 (t, J=7.6 Hz, 2H), 2.66-2.58 (m, 2H), 2.53 (s, 3H), 2.24 (s, 3H).

HPLC (Grace Alltima C18 5 μm column, 25×4.6 mm) indicated that the product was 100% pure (retention time 10.2 min; gradient: 20 to 100% MeCN/0.1% TFA in water/0.1% TFA 20 min measured at a wavelength of 488 nm).

### 4.4.5. Synthesis of [BODIPY FL-PEG₂-py-PtCl((1R,2R)-(−)-1,2-diaminocyclohexane)]⁺ TFA⁻ (4d)

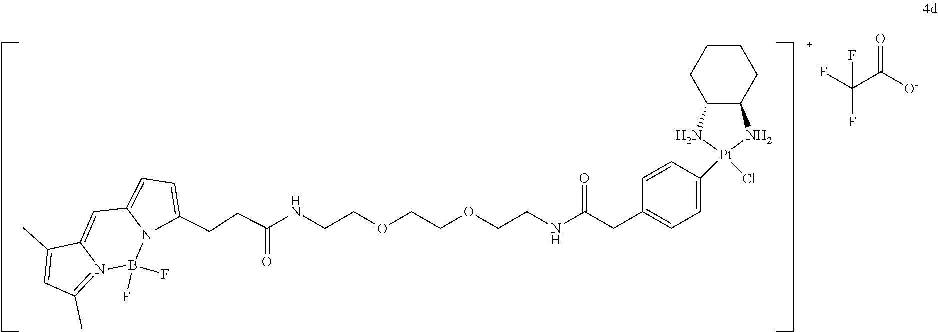

PtCl₂((1R,2R)-(−)-1,2-diaminocyclohexane) (1a) (50 mg, 131 μmol) and AgNO₃ (26 mg, 153 μmol) were dissolved in dry DMF (10 mL) under argon atmosphere and stirred for 22 h at room temperature under light exclusion (the reaction flask has been darkened). Subsequently, the mixture was filtered through a 0.2 μm syringe filter, to give a 13.2 mM stock solution of activated Pt-complex. Then, to the solution of BODIPY FL-PEG₂-py (L2) (14 mg, 26 μmol, 1.0 eq.) in DMF (200 μL), the 13.2 mM stock solution of activated Pt-complex (5.20 mL, 68.4 μmol, 2.6 eq.) was added, followed by triethylamine (7.21 μL, 52 μmol, 2.0 eq.), and the course of the reaction was followed by HPLC. The reaction mixture was stirred for 5 h at room temperature under light exclusion (the reaction flask has been darkened). At this moment, the reaction mixture contained 64.7% product and no starting material.

The mixture was concentrated under reduced pressure, diluted with water/MeOH (2.5:1, 2.5 mL), and filtered through a 0.2 μm syringe filter. Purification was performed by preparative reverse-phase HPLC (Grace Alltima C18 5 μm column, 22×250 mm; gradient: 35 to 85% MeOH/0.1% TFA in water/0.1% TFA in 36 min). Product fractions were lyophilized resulting in a bright orange solid (13 mg, 50% yield).

HRMS (ESI⁺) C₃₃H₄₈B₃₅ClF₂N₇O₄¹⁹⁵Pt [M]⁺ calc 885.3160, found 885.3162. HPLC (Grace Alltima C18 5 μm column, 25×4.6 mm) indicated that the product was 93.6% pure (retention time 12.2 min; gradient: 20 to 100% MeCN/0.1% TFA in water/0.1% TFA in 20 min measured at a wavelength of 488 nm).

### Example 5: Examples of Bromido Lx-“Semi-Final Products” Br-Lx-PFM (Bromido SFMs)

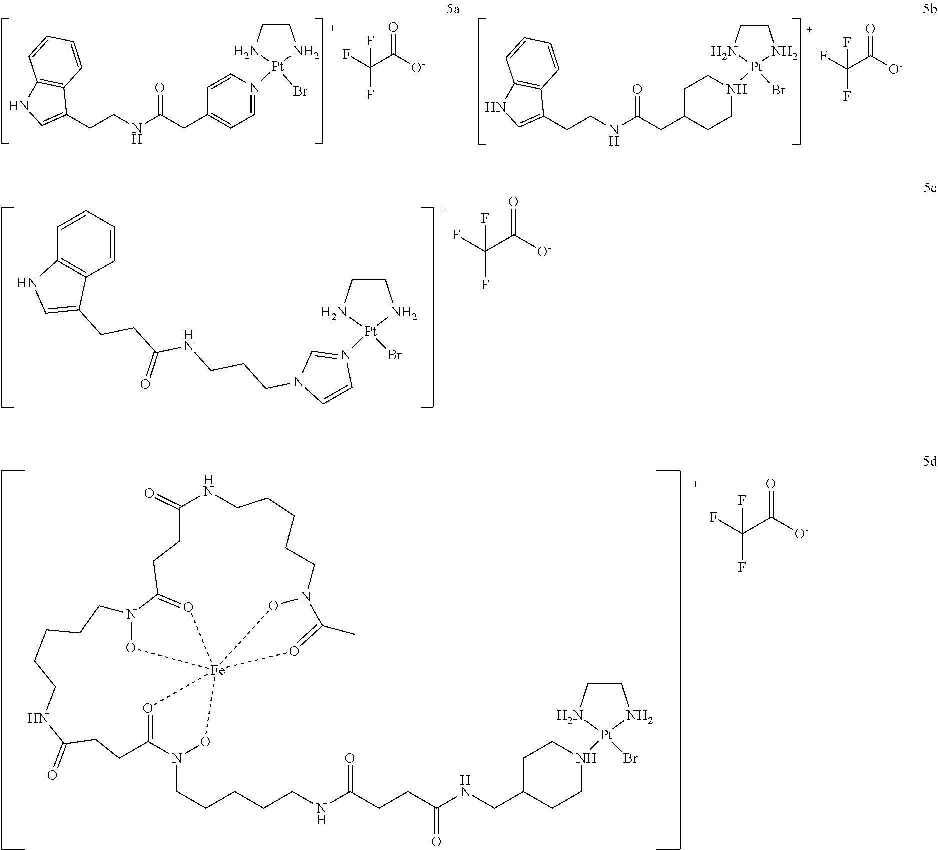

### 5.1. Synthesis and Analytical Characterization of [ind-py-PtBr(ethane-1,2-diamine)]⁺ TFA⁻ (5a)

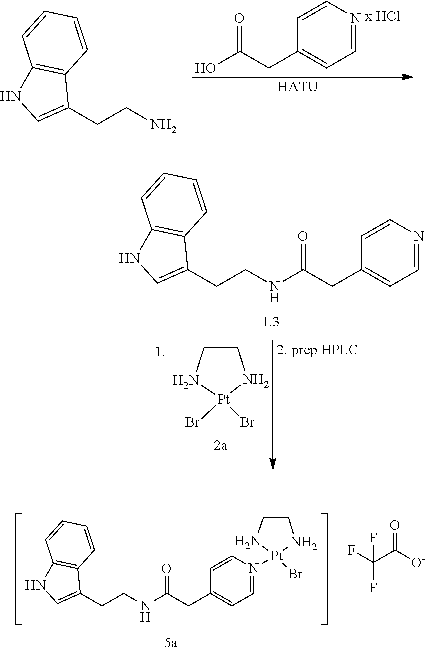

### 5.1.1. Synthesis of the Ligand N-(2-(1H-indol-3-yl)ethyl)-2-(pyridin-4-yl)acetamide (ind-py, L3)

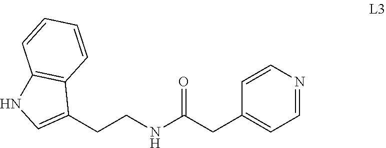 [SMILES: O=C(Cc1ccncc1)NCCc1c[nH]c2ccccc12]

2-(Pyridin-4-yl)acetic acid hydrochloride (365 mg, 2.0 mmol) was suspended in dry DMF (5 mL) and tryptamine (392 mg, 2.4 mmol) was added, followed by the addition of HATU (1.16 g, 4.0 mmol) and DIPEA (1.4 mL, 8 mmol). After stirring at room temperature for 24 h, the mixture was diluted with water, extracted with DCM, and after removal of solvents under reduced pressure the residue was absorbed on Celite and purified chromatographically on silica (eluent: DCM/MeOH/NH_{3aq.}=100:1:1 to 100:2:1 to 100:3:1). After drying, an orange glass (388 mg, 70% yield) was obtained.

HRMS (ESI⁺) C₁₇H₁₈N₃O [M+H]⁺ calc 280.1460, found 280.1444.

HPLC (Grace Alltima C18 5 μm column, 25×4.6 mm) indicated that the product was 98.5% pure (retention time 14.9 min; gradient: 5 to 50% MeCN/0.1% TFA in water/0.1% TFA in 18 min measured at a wavelength of 273 nm).

### 5.1.2. Synthesis of the Complex [ind-py-PtBr(ethane-1,2-diamine)]⁺ TFA⁻ (5a)

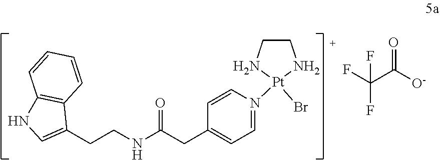

N-(2-(1H-Indol-3-yl)ethyl)-2-(pyridin-4-yl)acetamide (L3) (ind-py; 14.0 mg, 50 μmol, 1.0 eq.) and PtBr₂(ethane-1,2-diamine) (2a) (31.1 mg, 75 μmol, 1.5 eq.) were dissolved in dry DMF (500 μL) under argon atmosphere. Triethylamine (10.5 μL, 75 μmol, 1.5 eq.) was added and the course of the reaction was followed by HPLC. The reaction mixture was stirred at 60° C. for 42 h, then the temperature was increased to 70° C. and the reaction mixture was stirred for an additional 20 h. At this moment, the reaction mixture contained 94.4% product and 1.2% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (12.9 mg, 35.5% yield).

HRMS (ESI⁺) C₁₉H₂₅⁷⁹BrN₅O¹⁹⁵Pt [M]⁺ calc 613.0886, found 613.0877. HPLC (Grace Alltima C18 5 μm column, 25×4.6 mm) indicated that the product was 98.8% pure (retention time 17.8 min; gradient: 5 to 50% MeCN/0.1% TFA in water/0.1% TFA in 18 min measured at a wavelength of 223 nm).

### 5.2. Synthesis and Analytical Characterization of ind-pip-PtBr(ethane-1,2-diamine) (5b)

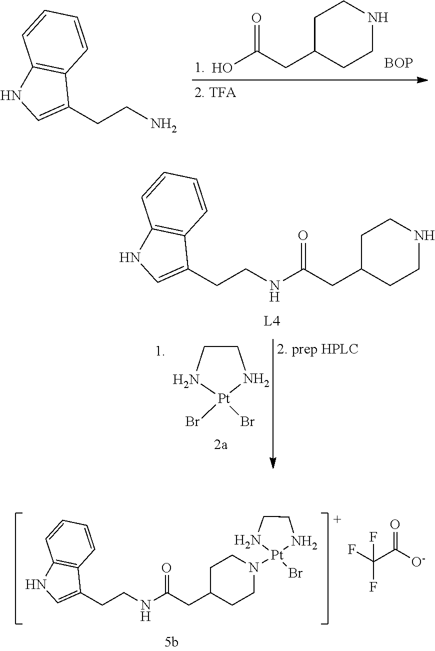

### 5.2.1. Synthesis of the Ligand N-(2-(1H-indol-3-yl)ethyl)-2-(piperidin-4-yl)acetamide (ind-pip, L4)

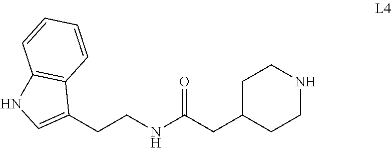 [SMILES: O=C(CC1CCNCC1)NCCc1c[nH]c2ccccc12]

Tryptamine (491 mg, 3.0 mmol, 1.0 eq.) was dissolved in DMF (5 mL). BOP (1.37 g, 3.0 mmol, 1.0 eq.), dissolved in DMF (5 mL), and DIPEA (523 μL, 3.0 mmol, 1.0 eq.) were added, followed by the addition of a solution of 2-(1-(tert-butoxycarbonyl)piperidin-4-yl)acetic acid (745 mg, 3.0 mmol, 1.0 eq.) in DMF (5 mL). After stirring at room temperature for 24 h, the mixture was diluted with water (15 mL), extracted with DCM (3×15 mL), and after removal of solvents under reduced pressure the residue was absorbed on Celite and purified chromatographically on silica using ethyl acetate/cyclohexane 1:1 as an eluent. After drying under reduced pressure, a brown oil (˜2.1 g) was obtained.

TFA (5 mL) was added to the material and the mixture was stirred at room temperature for 30 min, after which it was added slowly into an ice/water cooled 1 N NaOH (50 mL) solution. DCM was added and the mixture was stirred at 0° C. After addition of a small amount of MeOH the phases were separated and the aqueous layer was extracted with dichloromethane (9×25 mL). After evaporation, the residue (˜1.2 g of a brown oil) was absorbed on Celite and purified chromatographically on silica (eluent: isopropanol/NH_{3aq.}=100:1 to 100:2 to 100:3 to 100:4). The obtained material was then recrystallized from MeOH/dichloromethane/n-pentane and after drying a colorless solid (204 mg, 24% yield) was obtained.

HRMS (ESI⁺) C₁H₂₄N₃O [M+H]⁺ calc 286.1914, found 286.1920.

¹H NMR (400 MHz, DMSO-d₆): δ 10.80 (s, 1H, NH), 7.93-7.87 (m, 1H, NH), 7.55-7.50 (m, 1H), 7.35-7.31 (m, 1H), 7.12 (d, J=1.7 Hz, 1H), 7.09-7.03 (m, 1H), 7.00-6.94 (m, 1H), 3.36-3.28 (m, 2H), 2.94-2.84 (m, 2H), 2.84-2.77 (m, 2H), 2.48-2.38 (m, 2H), 2.00-1.93 (m, 2H), 1.85-1.66 (m, 1H), 1.58-1.46 (m, 2H), 1.15-0.94 (in, 2H).

HPLC (Grace Alltima C18 5 μm column, 25×4.6 mm) indicated that the product was 100% pure (retention time 15.1 min; gradient: 5 to 50% MeCN/0.1% TFA in water/0.1% TFA in 18 min measured at a wavelength of 273 nm).

### 5.2.2. Synthesis of the Complex [ind-pip-PtBr(ethane-1,2-diamine)]⁺ TFA⁻ (5b)

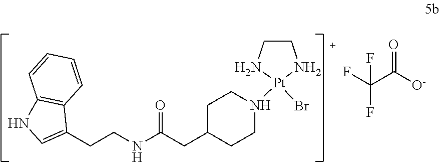

N-(2-(1H-Indol-3-yl)ethyl)-2-(piperidin-4-yl)acetamide (L4) (ind-pip; 14.3 mg, 50 μmol, 1.0 eq.) and PtBr₂(ethane-1,2-diamine) (2a) (20.8 mg, 50 μmol, 1.0 eq.) were dissolved in dry DMF (333 μL) under argon atmosphere. Triethylamine (6.98 μL, 50 μmol, 1.0 eq.) was added and the course of the reaction was followed by HPLC. The reaction mixture was stirred at 60° C. for 42 h. At this moment, the reaction mixture contained 88.6% product and maximally 2.6% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (9.0 mg, 24.5% yield). HRMS (ESI⁺) C₁₉H₃₁⁷⁹BrN₅O₁₉₅Pt [M]⁺ calc 619.1355, found 619.1353.

HPLC (Grace Alltima C18 5 μm column, 25×4.6 mm) indicated that the product was 95.6% pure (retention time 17.4 min; gradient: 5 to 50% MeCN/0.1% TFA in water/0.1% TFA in 18 min measured at a wavelength of 223 nm).

### 5.3. Synthesis and Analytical Characterization of ind-imi-PtBr(ethane-1,2-diamine) (5c)

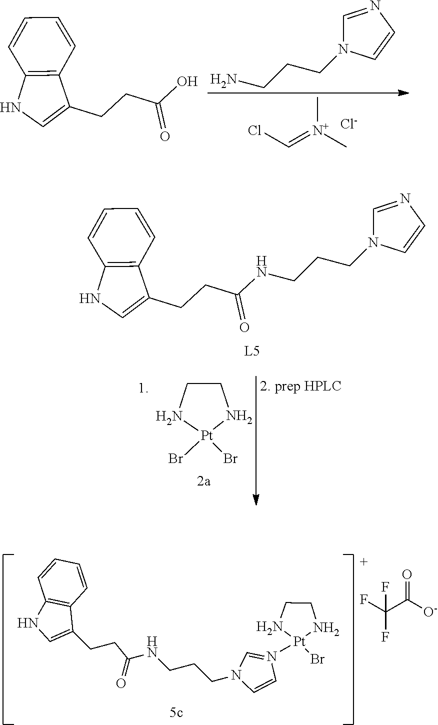

### 5.3.1. Synthesis of the Ligand N-(3-(1H-imidazol-1-yl)propyl)-3-(1H-indol-3-yl)propanamide (ind-imi, L5)

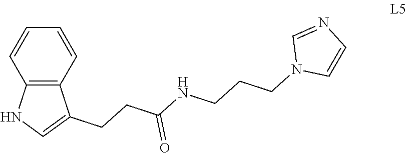 [SMILES: O=C(CCc1c[nH]c2ccccc12)NCCCn1ccnc1]

3-(1H-Indol-3-yl)propanoic acid (398 mg, 2.0 mmol, 1.0 eq.) was dissolved in dry DMF (5 mL) and N-(chloromethylene)-N-methylmethanaminium chloride (267 mg, 2.0 mmol, 1.0 eq.) was added at room temperature and stirred for 30 min at 40° C. Then, after cooling to room temperature and stirring for 1.5 h, 3-(1H-imidazol-1-yl)propan-1-amine (243 μL, 2.0 mmol, 1.0 eq.) was added, followed by the addition of DIPEA (1.7 mL, 10.0 mmol, 5.0 eq.). After stirring at room temperature for 22 h, the mixture was diluted with water, extracted with DCM, and after removal of solvents under reduced pressure the residue was absorbed on Celite and purified chromatographically on silica (eluent: DCM/MeOH/NH_{3aq.}=100:1:1 to 100:2:1 to 100:3:1 to 100:4:1) as an. After drying, a yellow oil (383 mg, 65% yield) was obtained.

HRMS (ESI⁺) C₁₇H₂₁N₄O [M+H]⁺ calc 297.1710, found 297.1697.

¹H NMR (400 MHz, DMSO-d₆): δ 10.77 (s, 1H, NH), 7.92-7.86 (m, 1H, NH), 7.56 (s, 1H), 7.55-7.51 (m, 1H), 7.34-7.30 (m, 1H), 7.12 (s, 1H), 7.11-7.08 (m, 1H), 7.08-7.02 (m, 1H), 6.99-6.94 (m, 1H), 6.87 (s, 1H), 3.85 (t, J=6.9 Hz, 2H), 3.04-2.96 (m, 2H), 2.93 (t, J=7.6 Hz, 2H), 2.45 (t, J=7.6 Hz, 2H), 1.77 (quint, J=6.8 Hz, 2H).

HPLC (Grace Alltima C18 5 μm column, 25×4.6 mm) indicated that the product was 100% pure (retention time 14.5 min; gradient: 5 to 50% MeCN/0.1% TFA in water/0.1% TFA in 18 min measured at a wavelength of 273 nm).

### 5.3.2. Synthesis of the Complex [ind-imi-PtBr(ethane-1,2-diamine)]⁺ TFA⁻ (5c)

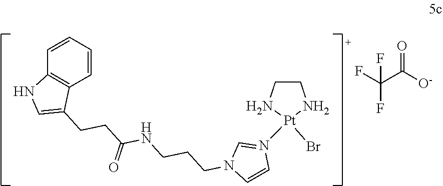

N-(3-(1H-Imidazol-1-yl)propyl)-3-(1H-indol-3-yl)propanamide (L5) (ind-imi; 14.8 mg, 50 μmol, 1.0 eq.) and PtBr₂(ethane-1,2-diamine) (2a) (31.1 mg, 75 μmol, 1.5 eq.) were dissolved in dry DMF (500 μL) under argon atmosphere. Triethylamine (10.5 μL, 75 μmol, 1.5 eq.) was added and the course of the reaction was followed by HPLC. The reaction mixture was stirred at 60° C. for 20 h, then the temperature was increased to 70° C. and the reaction mixture was stirred for an additional 20 h. At this moment, the reaction mixture contained 53.9% of the desired product and 5.2% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (7.7 mg, 20.7% yield).

HRMS (ESI⁺) C₁₉H₂₈⁷⁹BrN₆O¹⁹⁵Pt [M]⁺ calc 630.1151, found 630.1140.

HPLC (Grace Alltima C18 5 μm column, 25×4.6 mm) indicated that the product was 98.8% pure (retention time 17.2 min; gradient: 5 to 50% MeCN/0.1% TFA in water/0.1% TFA in 20 min measured at a wavelength of 223 nm).

### 5.4. Synthesis and Analytical Characterization of [(Fe)DFO-pip-PtBr(ethane-1,2-diamine)]⁺

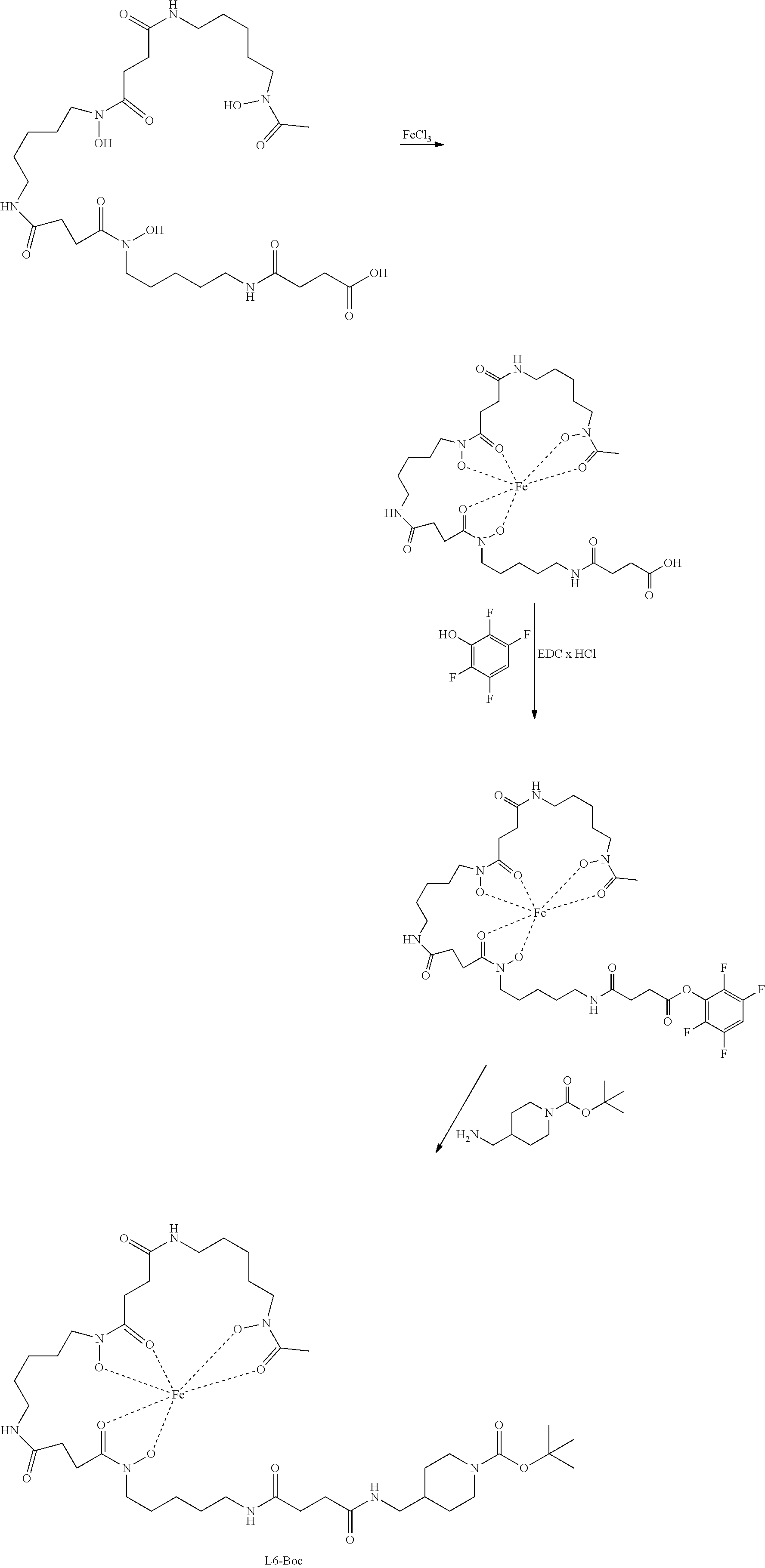

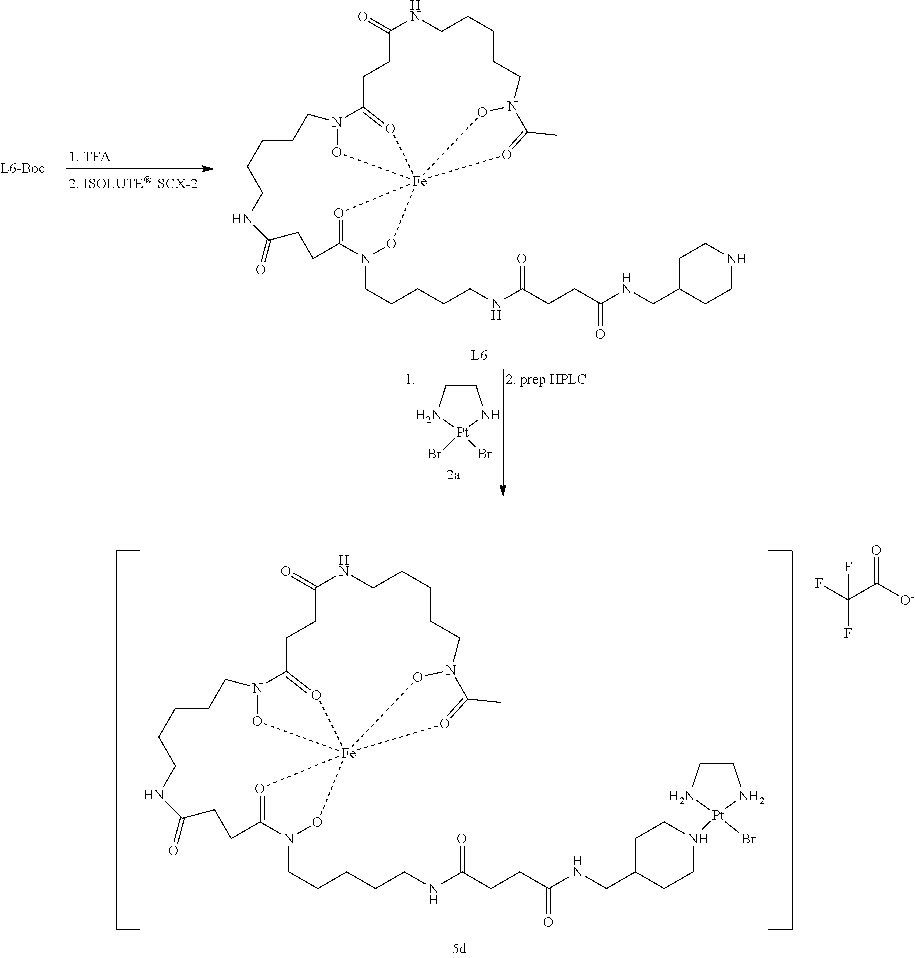

### 5.4.1. Synthesis of (Fe)DFO-suc

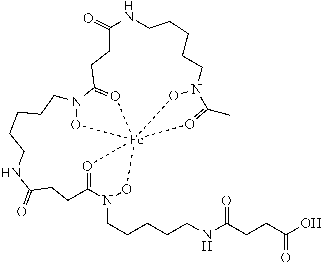

The procedure was adapted from Vugts et at, Bioconjugate Chem. 2011, 22, 2072-2081.

A solution of FeCl₃ (400 mg/mL in 0.5 M HCl) was prepared and 90 μL of this solution was added dropwise to a mixture of N-succinyl Desferal (DFO-suc, 120 mg, 182 μmol) in 0.1 M Na₂CO₃ (2.64 mL) and 0.9% NaCl (2.31 mL). The resulting mixture was stirred at room temperature for 10 min. The reaction mixture was used in the next step without further workup or purification.

### 5.4.2. Synthesis of (Fe)DFO-suc-TFP

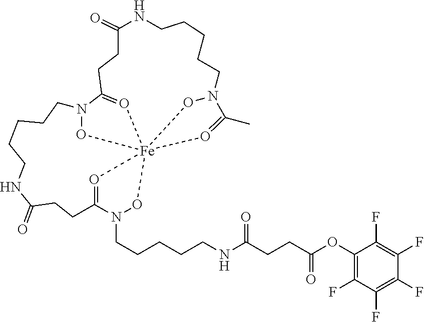

The procedure was adapted from Vugts et al., Bioconjugate Chem. 2011, 22, 2072-2081.

To the reaction mixture containing (Fe)DFO-suc (130 mg, 182 mot) were added 0.9% NaCl (5 mL), MeCN (1.8 mL) and 2,3,5,6-tetrafluorophenol (290 mg, 1.75 mmol) in MeCN (200 μL). Next, EDC×HCl (550 mg, 2.87 mmol) was added and the mixture was stirred for 15 min. Subsequently, a second portion of EDC×HCl (500 mg, 2.61 mmol) was added and the mixture was stirred for another 15 min. The reaction mixture was divided into two equal batches and poured into 0.9% NaCl (30 mL each) and the resulting mixtures were trapped on two activated double Sep-Pak C18 Plus columns. These two double Sep-Pak C18 Plus columns were washed with water (3×20 mL each), and the product was eluted with 2×1.5 mL MeCN. Thus, two product batches were collected, each containing the product in −3 mL of solvents.

HPLC (Grace Alltima C18 5 μm column, 25×4.6 mm) indicated that batch 1 was 94.8% pure and batch 2 was 95.2% pure (retention time 20.4 min; gradient: 5 to 50% MeCN/0.1% TFA in water/0.1% TFA in 20 min measured at a wavelength of 430 nm). It was assumed that the yield was ˜80% (based on the results obtained by Vugts et al., Bioconjugate Chem. 2011, 22, 2072-2081). The two solutions containing product were used in the next step without further workup or purification.

### 5.4.3. Synthesis of (Fe)DFO-suc-pip-Boc (L6-Boc)

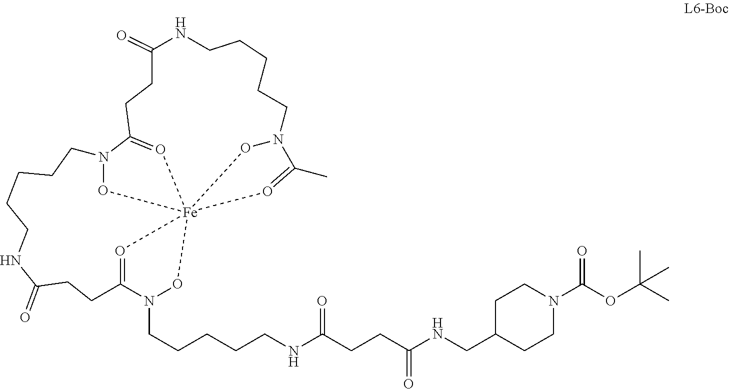

tort-Butyl 4-(aminomethyl)piperidine-1-carboxylate (23.5 mg, 110 μmol) was suspended in MeCN (300 μL) and the mixture was added to (Fe)DFO-suc-TFP (batch 2; −63 mg, 73 μmol in 3 mL MeCN; 95.2% purity). Subsequently, DIPEA (25.5 μL, 146 μmol) was added to the reaction mixture which was stirred at room temperature. HPLC (Grace Alltima C18 5 μm column, 25×4.6 mm) indicated that the product was >95% pure after stirring for 75 min (retention time 18.4 min; gradient: 5 to 50% MeCN/0.1% TFA in water/0.1% TFA in 20 min measured at a wavelength of 430 nm). The reaction mixture containing L6-Boc was evaporated and used in the next step without further purification.

### 5.4.4. Synthesis of the Ligand (Fe)DFO-suc-pip (L6)

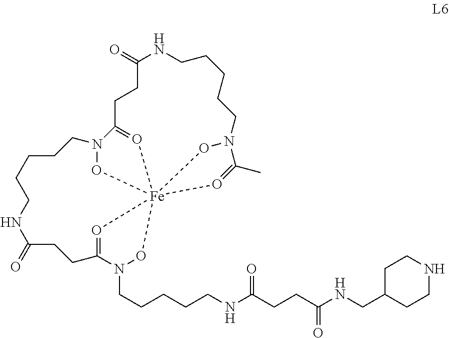

The crude material L6-Boc (˜67 mg, 73 μmol) was dissolved in DCM (3 mL), and TFA (3 mL) was added. The resulting mixture was stirred for 1.5 h at room temperature, concentrated, and the resulting residue was dissolved in MeOH. This dissolved material was loaded on an ISOLUTE® SCX-2 column that was activated with DCM. The column was washed with MeOH, and subsequently with 0.25 M NH_{3(aq)} in MeOH. The product was eluted with 1 M NH_{3(aq)} in MeOH and subsequently with 7 M NH_{3(aq)} in MeOH. The solvents were evaporated and the product was dissolved in water and lyophilized to afford a red solid (40.1 mg, 50.0 μmol, ˜55% over four steps from DFO-suc).

HRMS (ESI⁺) C₃₅H₆₂FeN₈O₁₀ [M+H]⁺ calc 810.3933, found 810.3928.

HPLC (Grace Alltima C18 5 μm column, 25×4.6 mm) indicated that the product was 97.5% pure (retention time 11.8 min; gradient: 5 to 50% MeCN/0.1% TFA in water/0.1% TFA in 20 min measured at a wavelength of 430 nm).

### 5.4.5. Synthesis of the Complex [(Fe)DFO-pip-PtBr(ethane-1,2-diamine)]⁺ TFA⁻ (5d)

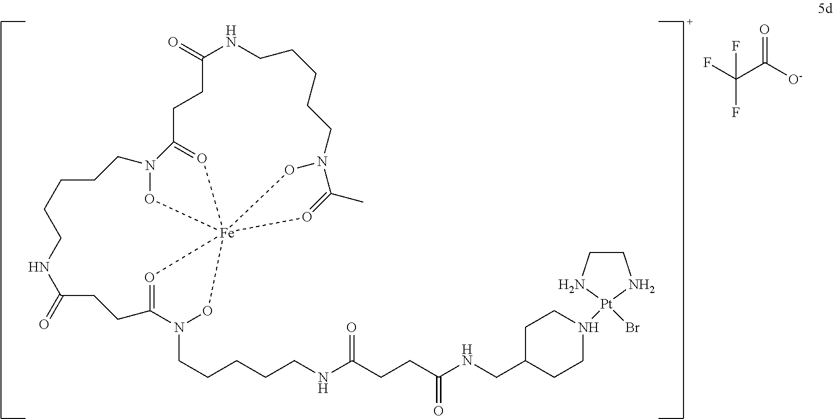

To an HPLC vial charged with L6 (16 mg, 20 μmol) were added DMF (200 μL), PtBr₂(ethane-1,2-diamine) (12.3 mg, 30 μmol), and TEA (4.13 μL, 30 μmol). The resulting mixture was shaken for 24 h at 60° C. The reaction mixture was diluted with water/MeOH (7:3, 3 mL) and filtered through a 0.2 μm syringe filter. Purification was performed by preparative reverse-phase HPLC (Grace Alltima C18 5 μm column, 22×250 mm; gradient: 30 to 50% MeOH/0.1% TFA in water/0.1% TFA in 36 min). Product fractions were collected and concentrated to ˜⅔ of the initial volume. Water (˜2 mL) was added and the mixture was lyophilized resulting in a red solid (14 mg, 56.3% yield). The product was dissolved in an aqueous 20 mM NaBr solution and stored as a 5 mM solution.

HRMS (ESI⁺) C₃₇H₆₉Fe₇₉BrN₁₀O₁₀¹⁹⁵Pt [M]⁺ calc 1143.3379, found 1143.3258. HPLC (Grace Alltima C18 5 μm column, 25×4.6 mm) indicated that the product was 95.6% pure (retention time 13.1 min; gradient: 5 to 50% MeCN/0.1% TFA in water/0.1% TFA in 20 min measured at a wavelength of 430 nm).

### Example 6: Examples of Iodido Lx-“Semi-Final Products” I-Lx-PFM (Iodido SFMs)

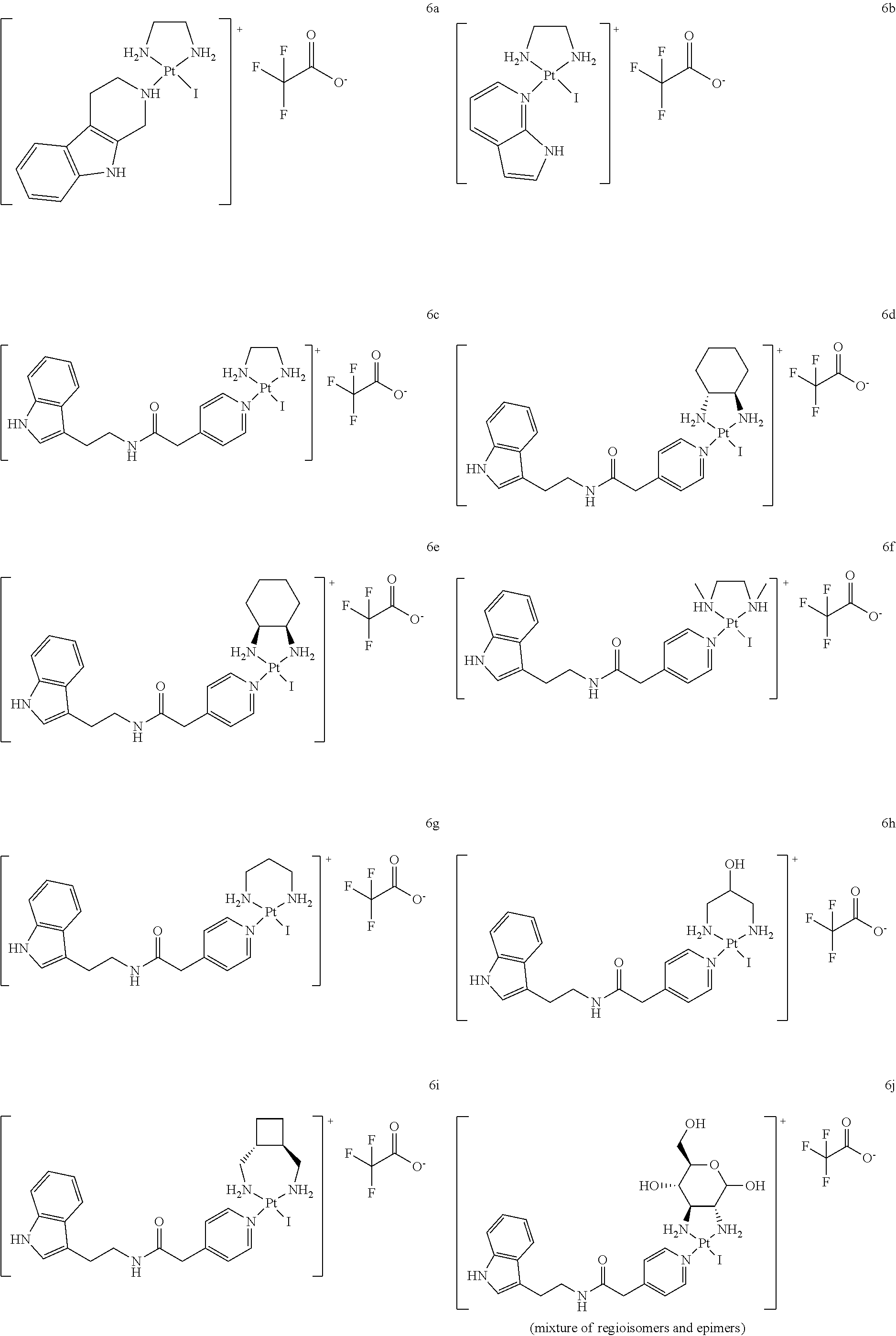

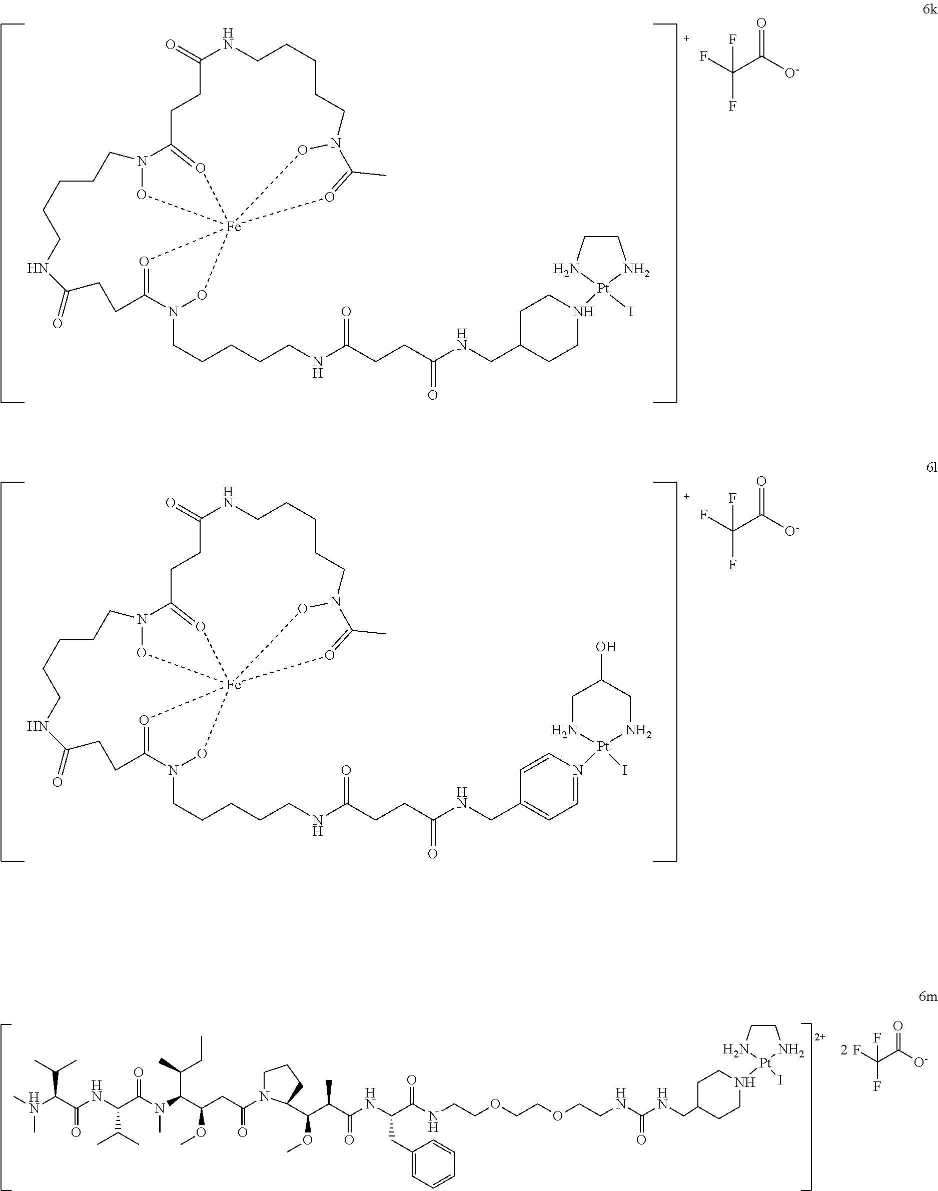

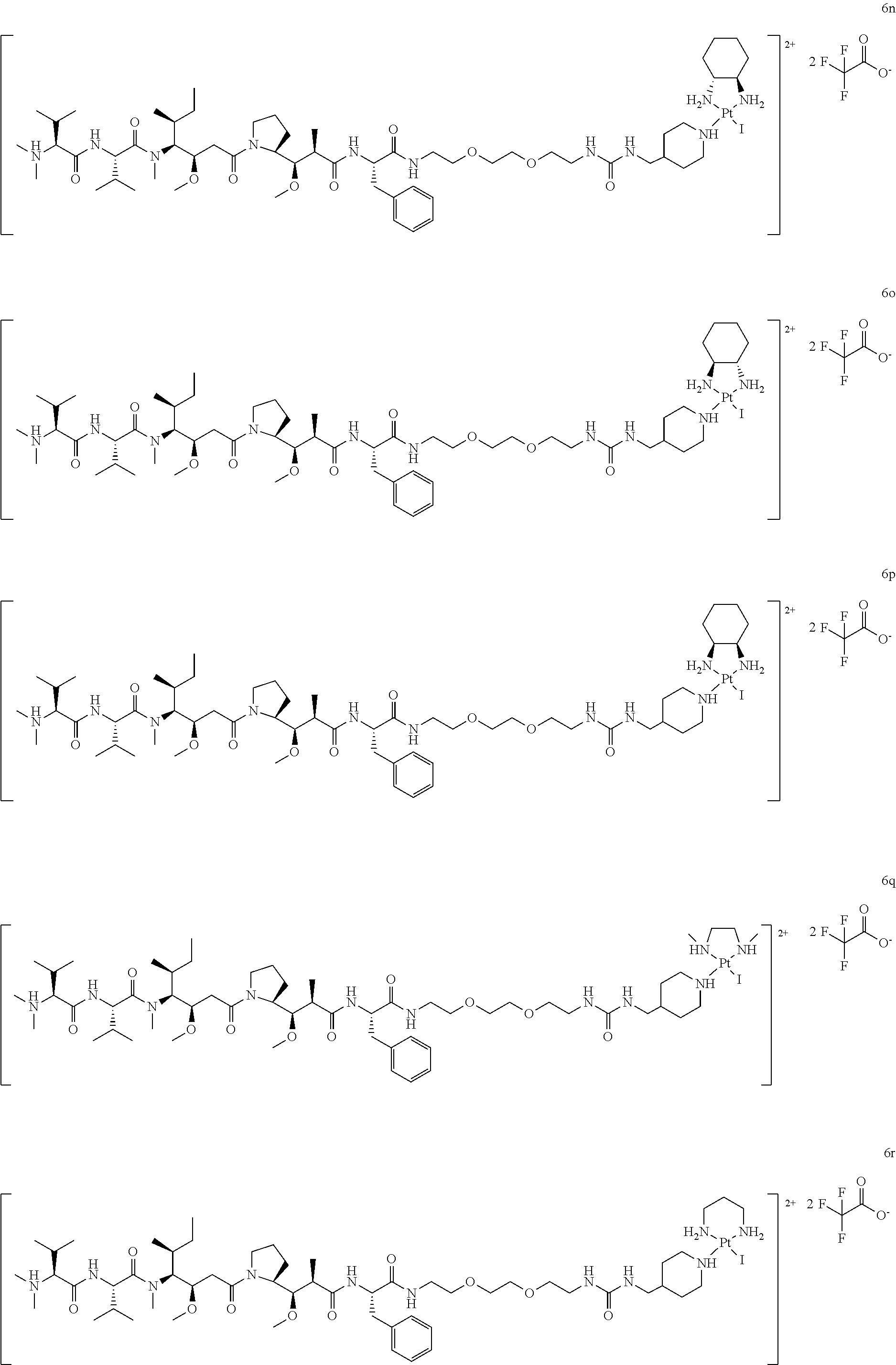

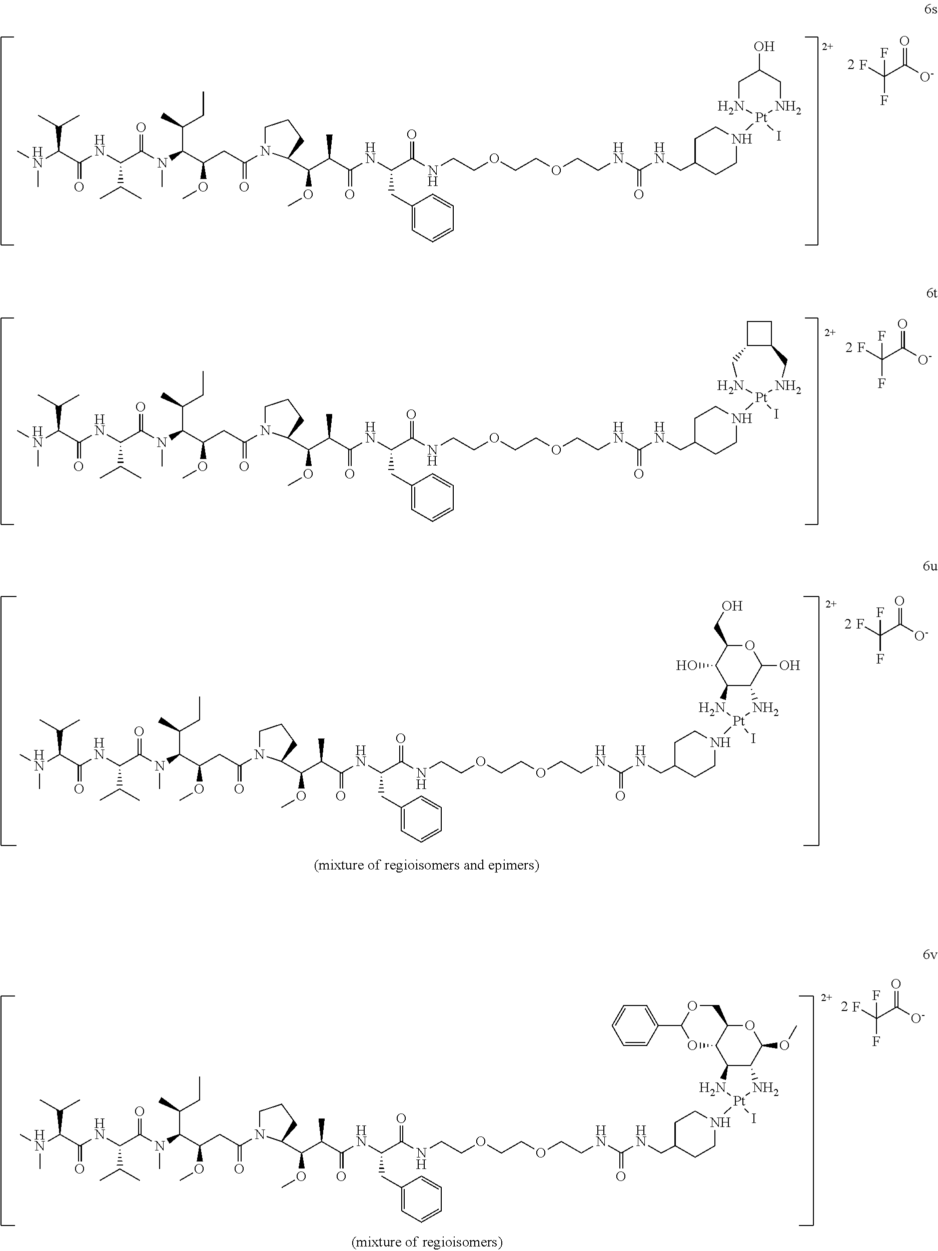

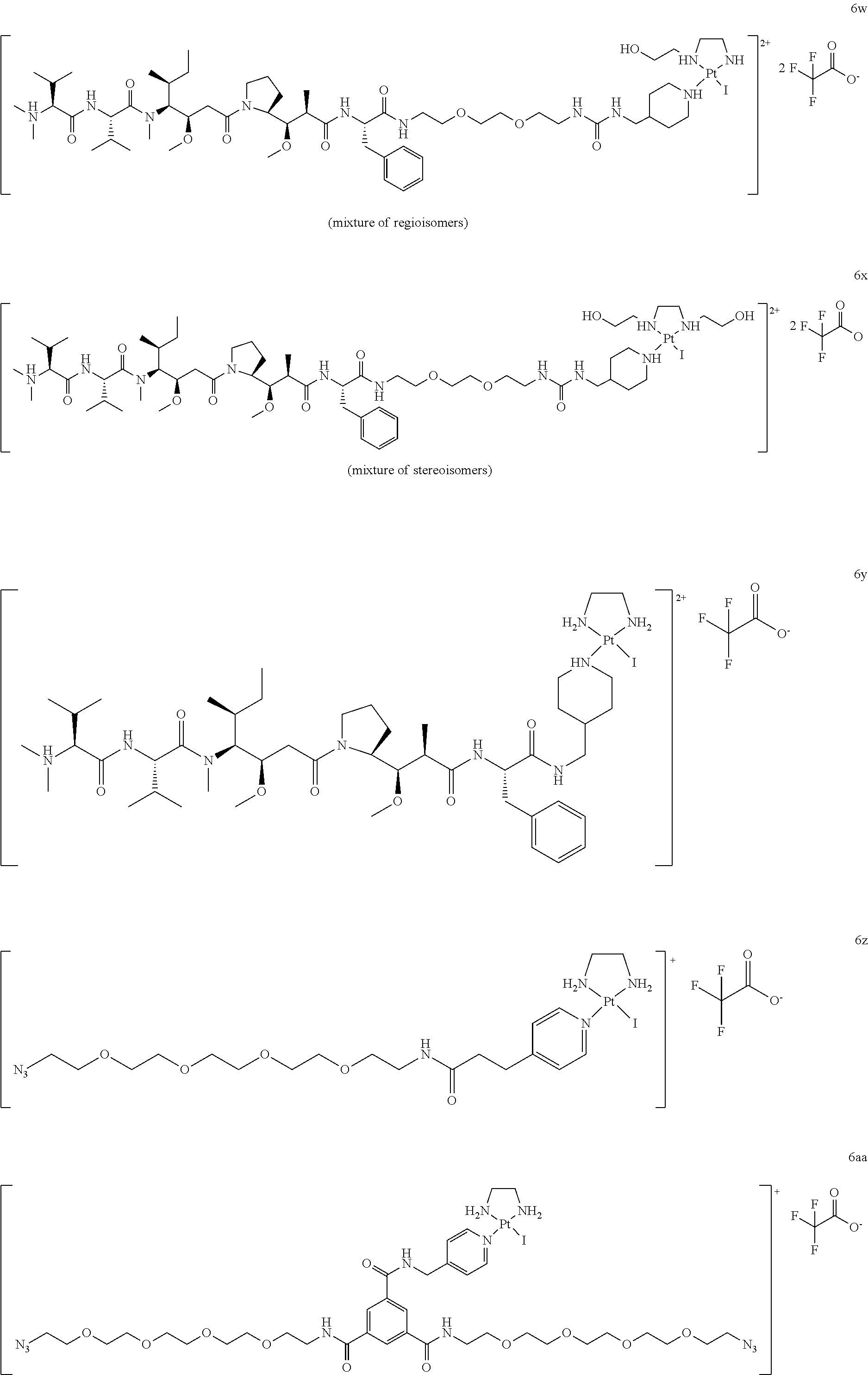

### 6.1. Synthesis and Analytical Characterization of [noreleagnine-Pt(ethane-1,2-diamine)I]⁺ TFA⁻ (6a)

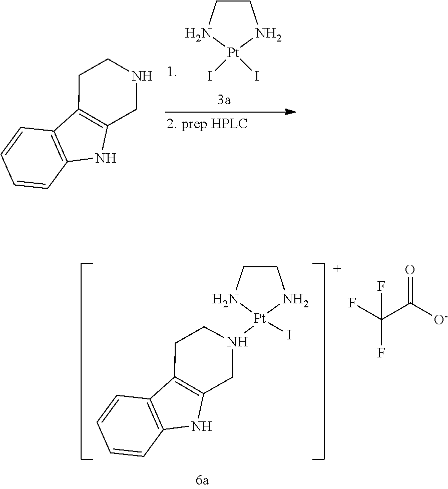

2,3,4,9-Tetrahydro-1H-pyrido[3,4-b]indole (noreleagnine; 9.1 mg, 50 μmol, 1.0 eq.) and Pt(ethane-1,2-diamine)I₂ (3a) (25.4 mg, 50 μmol, 1.0 eq.) were dissolved in dry DMF (333 μmol). Triethylamine (6.98 μL, 50 μmot, 1.0 eq.) was added and the course of the reaction was followed by HPLC. The reaction mixture was stirred at 60° C. for 24 h. At this moment, the reaction mixture contained 84.1% of the desired product and 4.4% of starting material (retention time 14.4 min).

The reaction mixture was diluted with water/MeOH (19:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (19.9 mg, 59.6% yield).

HRMS (ESI⁺) C₁₃H₂₀IN₄¹⁹⁵Pt [M]⁺ calc 554.0376, found 554.0369.

HPLC (Grace Alltima C18 5 μm column, 25×4.6 mm) indicated that the product was 97.9% pure (retention time 19.9 min; gradient: 5 to 50% MeCN/0.1% TFA in water/0.1% TFA in 18 min measured at a wavelength of 273 nm).

### 6.2. Synthesis and Analytical Characterization of [7-azaindole-Pt(ethane-1,2-diamine)I]⁺ TFA⁻ (6b)

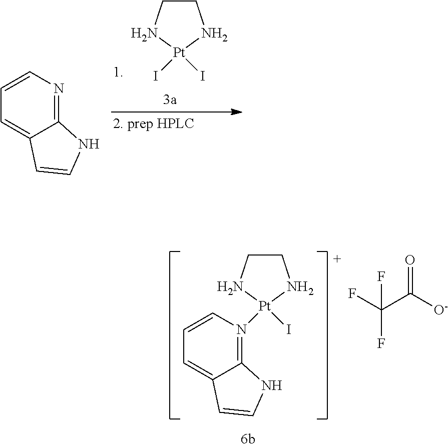

1H-Pyrrolo[2,3-h]pyridine (7-azaindole; 6.0 mg, 50 μmol, 1.0 eq.) and Pt(ethane-1,2-diamine)I₂ (3a) (25.4 mg, 50 μmol, 1.0 eq.) were dissolved in dry DMF (333 μmol). Triethylamine (6.98 μL, 50 μmol, 1.0 eq.) was added and the course of the reaction was followed by HPLC. The reaction mixture was stirred at 60° C. for 24 h. At this moment, the reaction mixture contained 72.8% of the desired product and 26.9% of starting material (retention time 4.5 min).

The reaction mixture was diluted with water/MeOH (19:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (12.2 mg, 39.8% yield).

HRMS (ESI⁺) C₉H₁₄IN₄¹⁹⁵Pt [M]⁺ calc 499.9906, found 499.9910.

HPLC (Grace Alltima C18 5 μm column, 25×4.6 mm) indicated that the product was 99.5% pure (retention time 14.8 min; gradient: 5 to 50% MeCN/0.1% TFA in water/0.1% TFA in 18 min measured at a wavelength of 273 nm).

### 6.3. Synthesis and Analytical Characterization of [ind-py-Pt(ethane-1,2-diamine)I]⁺ TFA⁻ (6c)

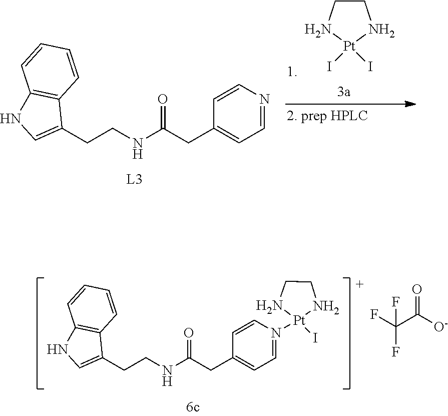

N-(2-(1H-Indol-3-yl)ethyl)-2-(pyridin-4-yl)acetamide (L3) (ind-py; 14.0 mg, 50 μmol, 1.0 eq.) and Pt(ethane-1,2-diamine)I₂ (3a) (25.4 mg, 50 μmol, 1.0 eq.) were dissolved in dry DMF (333 μL) under argon atmosphere. Triethylamine (6.98 μL, 50 μmol, 1.0 eq.) was added and the course of the reaction was followed by HPLC. The reaction mixture was stirred at 60° C. for 23 5 h. At this moment, the reaction mixture contained 95.0% product and 5.0% starting material. The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (25.2 mg, 65.1% yield).

HRMS (ESI⁺) C₁₉H₂₅IN₅O¹⁹⁵Pt [M]⁺ calc 661.0747, found 661.0731.

HPLC (Grace Alltima C18 5 μm column, 25×4.6 mm) indicated that the product was 99.6% pure (retention time 18.8 min; gradient: 5 to 50% MeCN/0.1% TFA in water/0.1% TFA in 18 min measured at a wavelength of 273 nm).

### 6.4. Synthesis and Analytical Characterization of [ind-py-Pt(((1R,2R)-(−)-1,2-diaminocyclohexane))I]⁺ TFA⁻ (6d)

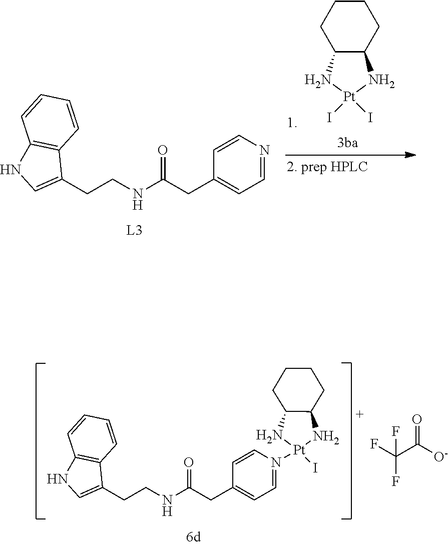

N-(2-(1H-Indol-3-yl)ethyl)-2-(pyridin-4-yl)acetamide (L3) (ind-py; 14.0 mg, 50 μmol, 1.0 eq.) and Pt(((1R,2R)-(−)-1,2-diaminocyclohexane))I₂ (3b) (42.2 mg, 75 μmol, 1.5 eq.) were dissolved in dry DMF (333 μL) under argon atmosphere. Triethylamine (10.46 μL, 75 μmol, 1.5 eq.) was added and the course of the reaction was followed by HPLC. The reaction mixture was stirred at 40° C. for 68 h and then at 50° C. for 24 h. At this moment, the reaction mixture contained 90.2% product and 4.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (19.7 mg, 47.6% yield).

HRMS (ESI⁺) C₂₃H₃₁IN₅O¹⁹⁵Pt [M]⁺ calc 715.1216, found 715.1194.

HPLC (Grace Alltima C18 5 μm column, 25×4.6 mm) indicated that the product was 99.6% pure (retention time 12.5 min; gradient: 20 to 100% MeCN/0.1% TFA in water/0.1% TFA in 20 min measured at a wavelength of 273 nm).

### 6.5. Synthesis and Analytical Characterization of [ind-py-Pt(cis-1,2-diaminocyclohexane)I]⁺ TFA⁻ (6e)

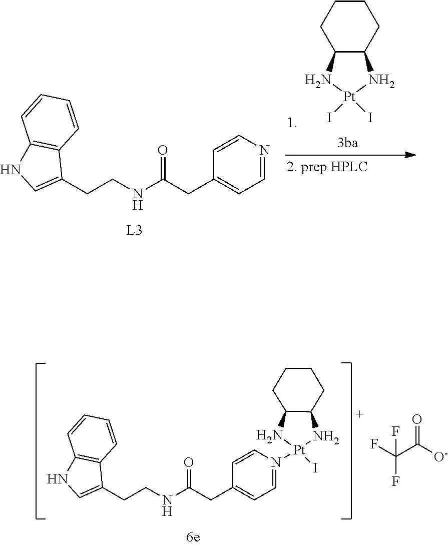

N-(2-(1H-Indol-3-yl)ethyl)-2-(pyridin-4-yl)acetamide (L3) (ind-py; 14.0 mg, 50 μmol, 1.0 eq.) and Pt(cis-1,2-diaminocyclohexane)I₂ (3d) (42.4 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 40° C. for 19 h. At this moment, the reaction mixture contained 88.4% product and 6.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (15.4 mg, 37.2% yield).

HRMS (ESI⁺) C₂₃H₃₁IN₅O¹⁹⁵Pt [M]⁺ calc 715.1216, found 715.1195.

HPLC (Grace Alltima C18 5 μm column, 25×4.6 mm) indicated that the product was 99.6% pure (retention time 12.3 min; gradient: 20 to 100% MeCN/0.1% TFA in water/0.1% TFA in 20 min measured at a wavelength of 273 nm).

### 6.6. Synthesis and Analytical Characterization of [ind-py-Pt(N¹,N²-dimethylethane-1,2-diamine)I]⁺ TFA⁻ (6f)

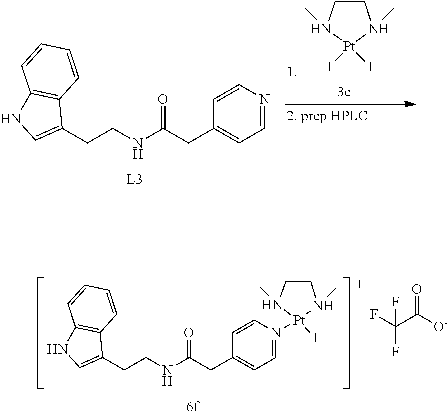

N-(2-(1H-Indol-3-yl)ethyl)-2-(pyridin-4-yl)acetamide (L3) (ind-py; 14.0 mg, 50 μmol, 1.0 eq.) and Pt(N¹,N²-dimethylethane-1,2-diamine)I₂ (3e) (40.3 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 40° C. for 20 h. At this moment, the reaction mixture contained 89.9% product and 11.5% starting material.

The reaction mixture was diluted with water/MeOH (4:1, 2.5 mL) and filtered through a 02 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (12.4 mg, 30.9% yield).

HRMS (ESI⁺) C₂₁H₂₉IN₅O¹⁹⁵Pt [M]⁺ calc 689.1060, found 689.1043.

HPLC (Grace Alltima C18 5 μm column, 25×4.6 mm) indicated that the product was 100% pure (retention time 11.6 min; gradient: 20 to 100% MeCN/0.1% TFA in water/0.1% TFA in 20 min measured at a wavelength of 223 nm).

### 6.7. Synthesis and Analytical Characterization of [ind-py-PtI(propane-1,3-diamine)]⁺ TFA⁻ (6g)

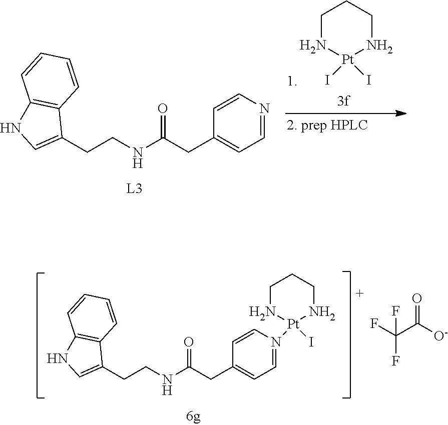

N-(2-(1H-Indol-3-yl)ethyl)-2-(pyridin-4-yl)acetamide (L3) (ind-py; 14.0 mg, 50 μmol, 1.0 eq.) and PtI₂(propane-1,3-diamine) (31) (39.2 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 25° C. for 16.5 h, then continued at 30° C. for 5 h, at 40° C. for 18 h, and finally at 50° C. for 5 h. At this moment, the reaction mixture contained 97.3% product and 2.7% starting material. The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (5.2 mg, 13.2% yield).

HRMS (ESI⁺) C₂₀H₂₇IN₅O¹⁹⁵Pt [M]⁺ calc 675.0903, found 675.0985.

HPLC (Grace Alltima C18 5 tin column, 25×4.6 mm) indicated that the product was 97.9% pure (retention time 19.6 min; gradient: 5 to 50% MeCN/0.1% TFA in water/0.1% TFA in 18 min measured at a wavelength of 223 nm).

### 6.8. Synthesis and Analytical Characterization of [ind-py-Pt(1,3-diaminopropan-2-ol)I]⁺ TFA⁻ (6h)

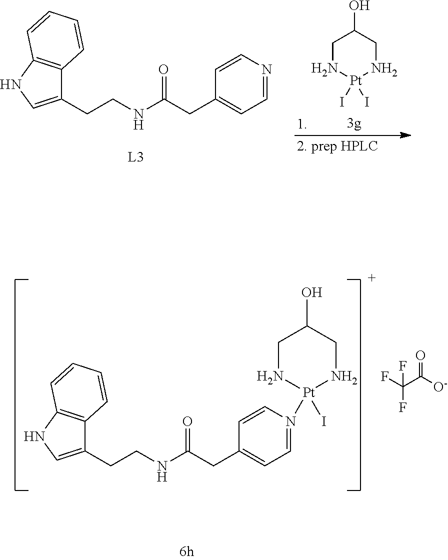

N-(2-(1H-Indol-3-yl)ethyl)-2-(pyridin-4-yl)acetamide (L3) (ind-py; 14.0 mg, 50 μmom, 1.0 eq.) and Pt(1,3-diaminopropan-2-ol)I₂ (3g) (40.4 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 25° C. for 16.5 h, then continued at 30° C. for 5 h, at 40° C. for 18 h, and finally at 50° C. for 5 h. At this moment, the reaction mixture contained 93.4% product and 2.1% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (16.1 mg, 40.0% yield).

HRMS (ESI⁺) C₂₀H₂₇IN₅O₂¹⁹⁵Pt [M]⁺ calc 691.0852, found 691.0960.

HPLC (Grace Alltima C18 5 μm column, 25×4.6 mm) indicated that the product was 97.9% pure (retention time 18.7 min; gradient: 5 to 50% MeCN/0.1% TFA in water/0.1% TFA in 18 min measured at a wavelength of 223 nm).

### 6.9. Synthesis and Analytical Characterization of [ind-py-Pt(((1R,2R)-cyclobutane-1,2-diyl)dimethanamine)I]⁺ TFA⁻ (6i)

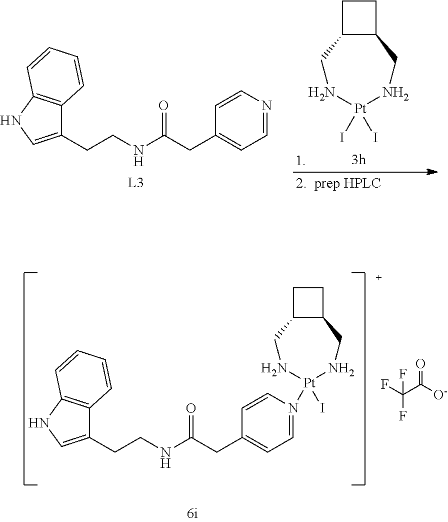

N-(2-(1H-Indol-3-yl)ethyl)-2-(pyridin-4-yl)acetamide (L3) (ind-py; 14.0 mg, 50 μmol, 1.0 eq.) and Pt(((1R,2R)-cyclobutane-1,2-diyl)dimethanamine)I₂ (3h) (42.2 mg, 75 μmom, 1.5 eq.) were dissolved in dry DMF (333 μL) under argon atmosphere. Triethylamine (10.45 μL, 75 μmom, 1.5 eq.) was added and the course of the reaction was followed by HPLC. The reaction mixture was stirred at 40° C. for 20 h. At this moment, the reaction mixture contained 69.3% product and 17.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 80% MeOH/0.1% TFA in water/0.1% TFA in 36 min). Product fractions were lyophilized resulting in a colorless solid (4.8 mg, 11.6% yield).

HRMS (ESI⁺) C₂₃H₃₁IN₅O¹⁹⁵Pt [M]⁺ calc 715.1216, found 715.1198.

HPLC (Grace Alltima C18 5 μm column, 25×4.6 mm) indicated that the product was 95.9% pure (retention time 13.2 min; gradient: 20 to 100% MeCN/0.1% TFA in water/0.1% TFA in 20 min measured at a wavelength of 273 nm).

### 6.10. Synthesis and Analytical Characterization of [ind-py-Pt((3R,4R,5S,6R)-3,4-diamino-6-(hydroxymethyl)tetrahydro-2H-pyran-2,5-diol)I]⁺ TFA⁻ (6j)

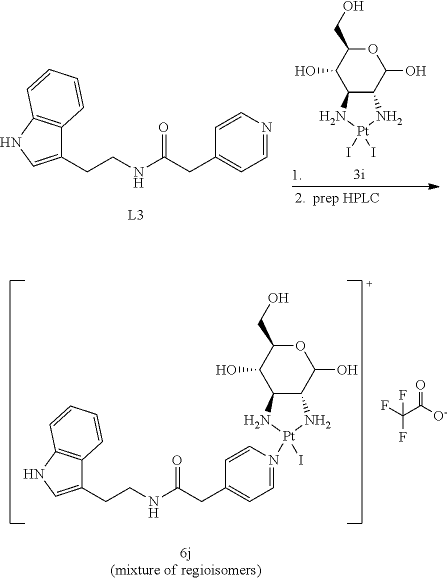

N-(2-(1H-Indol-3-yl)ethyl)-2-(pyridin-4-yl)acetamide (L3) (ind-py; 14.0 mg, 50 μmol, 1.0 eq.) and Pt((3R,4R,5S,6R)-3,4-diamino-6-(hydroxymethyl)tetrahydro-2H-pyran-2,5-diol)I₂ (3i) (47.0 mg, 75 μmol, 1.5 eq.) were dissolved in dry DMF (500 μL) under argon atmosphere. Triethylamine (10.45 μL, 75 μmol, 1.5 eq.) was added and the course of the reaction was followed by HPLC. The reaction mixture was stirred at 50° C. for 25 h. At this moment, the reaction mixture contained 82.6% product and 5.8% starting material.

The reaction mixture was diluted with 35% MeOH/water (2.0 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a beige solid (21.0 mg, 47.1% yield).

HRMS (ESI⁺) C₂₃H₃₁IN₅O₅¹⁹⁵Pt [M]⁺ calc 779.1013, found 779.1042.

HPLC (Grace Alltima C18 5 μm column, 25×4.6 mm) indicated that the product was 99.2% pure (note: the product was obtained as a mixture of regioisomers and epimers, so that several peaks were observed; retention times 16.8-17.6 min; gradient: 5 to 50% MeCN/0.1% TFA in water/0.1% TFA in 18 min measured at a wavelength of 273 nm).

### 6.11. Synthesis and Analytical Characterization of the complex [Pt((Fe)DFO-suc-pip)(ethane-1,2-diamine)I]⁺ TFA⁻ (6k)

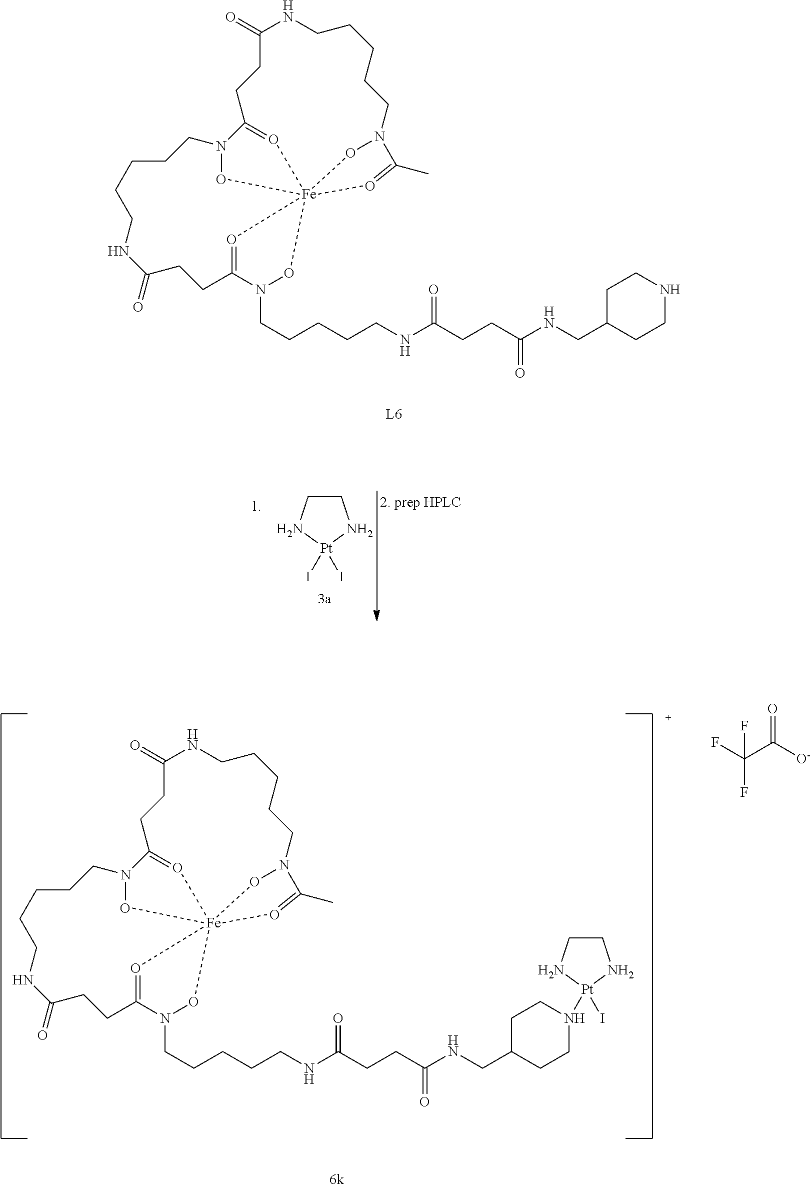

To an HPLC vial charged with (Fe)DFO-suc-pip (L6) (16 mg, 20 μmol, 1.0 eq.) were added DMF (200 μL), Pt(ethane-1,2-diamine)I₂ (3a) (15.1 mg, 30 μmol, 1.5 eq.), and TEA (4.13 μL, μmol, 1.5 eq.). The resulting mixture was shaken for 20 h at 60° C. The reaction mixture was diluted with water/MeOH (7:3, 3 mL) and filtered through a 0.2 μm syringe filter. Purification was performed by preparative reverse-phase HPLC (Grace Alltima C18 5 μm column, 22×250 mm; gradient: 30 to 50% MeOH/0.1% TFA in water/0.1% TFA in 36 min). Product fractions were collected and reduced to ˜⅔ of the initial volume. Water (˜5 mL) was added and the mixture was lyophilized resulting in a red solid (11 mg, 42.7% yield). The product was dissolved in an aqueous 20 mM NaI solution and stored as a 5 mM solution.

HRMS (ESI⁺) C₃₇H₆₉FeIN₁₀O₁₀¹⁹⁵Pt [M]⁺ calc 1191.3235, found 1191.3412.

HPLC (Grace Alltima C18 5 μm column, 25×4.6 mm) indicated that the product was 95.7% pure (retention time 13.8 min; gradient: 5 to 50% MeCN/0.1% TFA in water/0.1% TFA in 20 min measured at a wavelength of 430 nm).

### 6.12. Synthesis and Analytical Characterization of the complex [(Fe)DFO-suc-py-Pt(1,3-diaminopropan-2-ol)I]⁺ TFA⁻ (61)

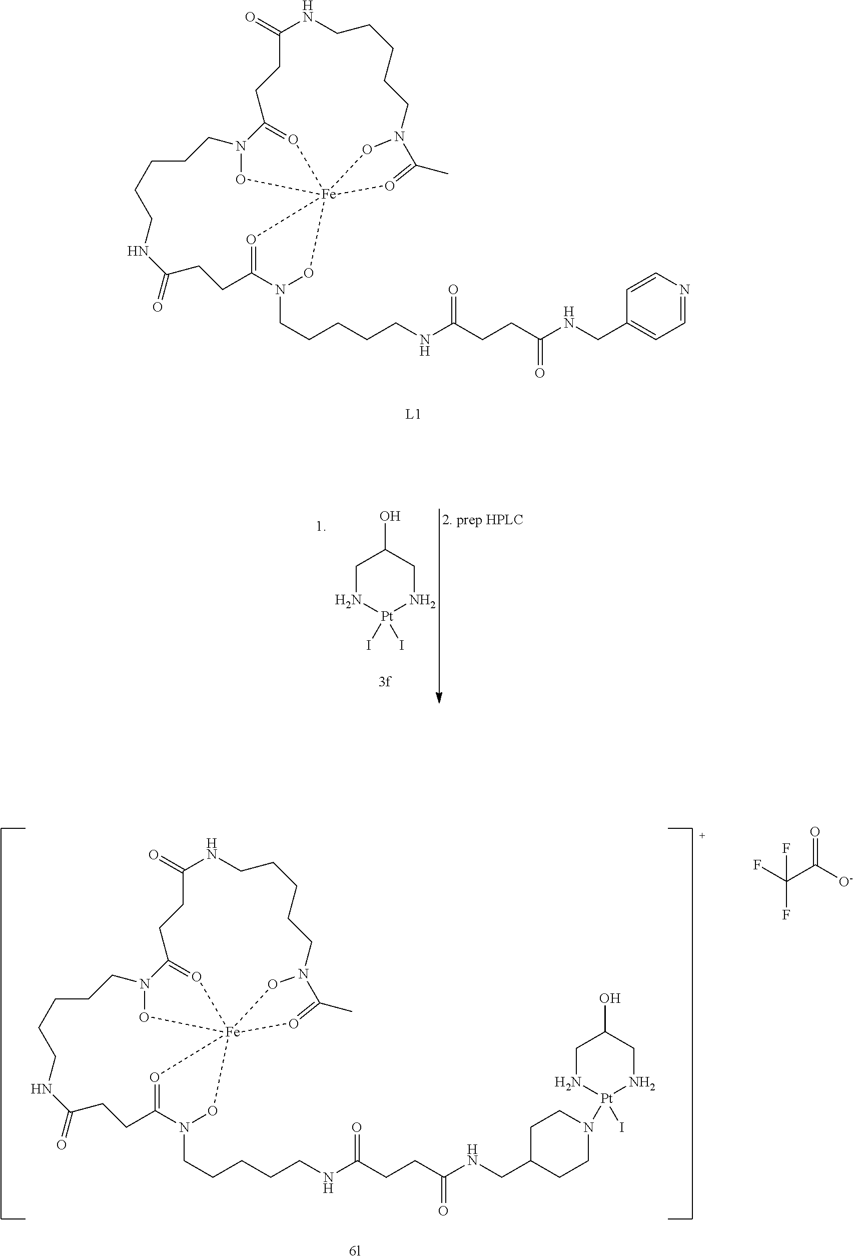

(Fe)DFO-suc-py (L1) (10.0 mg, 12 μmol, 1.0 eq.) and Pt(1,3-diaminopropan-2-ol)I₂ (31) (26.4 mg, 48 μmol, 4 eq.) were dissolved in dry DMF (375 μL) under argon atmosphere. Triethylamine (6.92 μL, 48 μmol, 4 eq.) was added and the course of the reaction was followed by HPLC. The reaction mixture was stirred at 40° C. for 16 h. At this moment, the reaction mixture contained 81.0% product and no starting material.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 30 to 55% MeOH/0.1% TFA in water/0.1% TFA in 36 min). Product fractions were lyophilized resulting in a colorless solid (7.6 mg, 46.0% yield).

HRMS (ESI⁺) C₃₈H₆₅FeIN₁₀NaO₁₁¹⁹⁵Pt [M+Na]²⁺ calc 619.1382, found 619.1328.

HPLC (Grace Alltima C18 5 μm column, 25×4.6 mm) indicated that the product was 96.0% pure (retention time 14.0 min; gradient: 5 to 50% MeCN/0.1% TFA in water/0.1% TFA in 20 min measured at a wavelength of 430 nm).

### 6.13. Synthesis and Analytical Characterization of the complex [AF-PEG₂-urea-pip-Pt(ethane-1,2-diamine)I]⁺ TFA⁻ (6m)

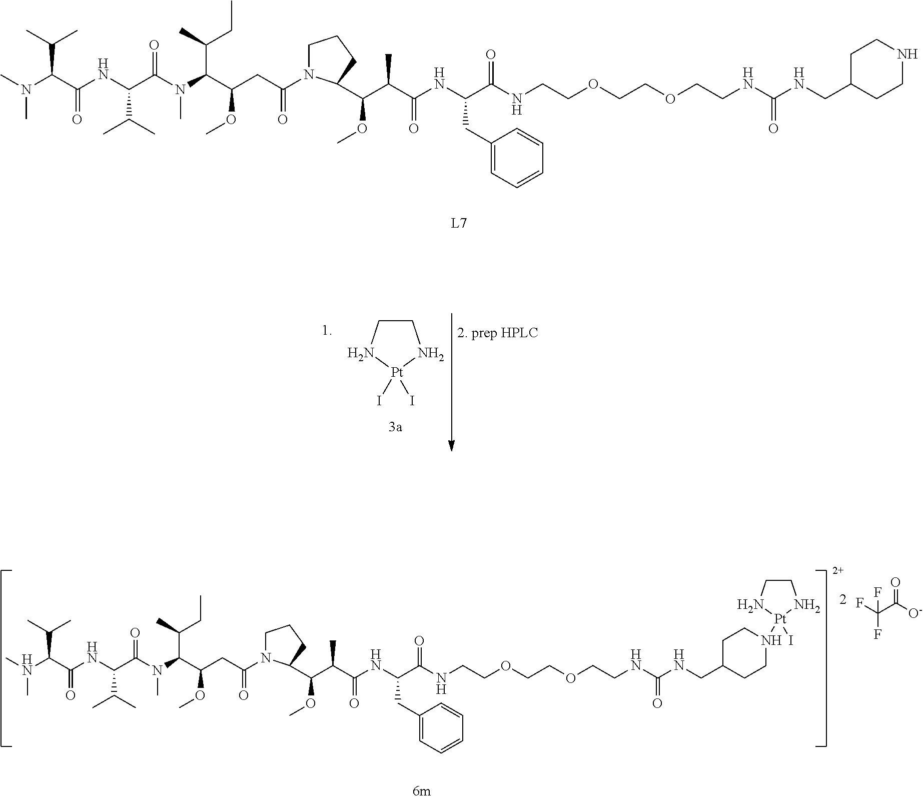

### 6.13.1. Synthesis of the Ligand AF-PEG₂-urea-pip (L7)

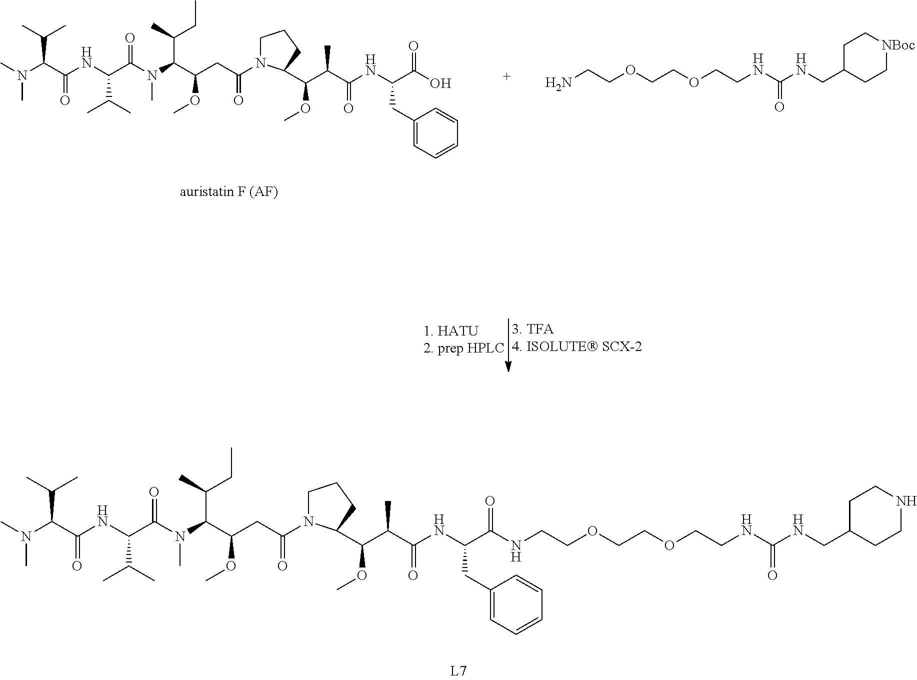 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C)C(C)C ; CC(C)(C)OC(=O)N1CCC(CNC(=O)NCCOCCOCCN)CC1 ; CC[C@H](C)[C@@H]([C@@H](CC(=O)N1CCC[C@H]1[C@H](OC)[C@@H](C)C(=O)N[C@@H](Cc1ccccc1)C(=O)NCCOCCOCCNC(=O)NCC1CCNCC1)OC)N(C)C(=O)[C@@H](NC(=O)[C@H](C(C)C)N(C)C)C(C)C]

Auristatin F (AF) (40.0 mg, 54 μmol, 1.0 eq.), dissolved in DMF (1.33 mL), was added to tert-butyl 4-(12-amino-3-oxo-7,10-dioxa-2,4-diazadodecyl)piperidine-1-carboxylate (62.5 mg, 161 μmol, 3.0 eq.; synthesis is described in Sijbrandi et al., Cancer Res. 2017, 72, 257-267) in DMF (1 mL). HATU (40.8 mg, 107 μmol, 2.0 eq.) and DIPEA (29 μL, 161 μmol, 3.0 eq.) were subsequently added and the mixture was stirred for 1.5 h in an ice bath. The reaction mixture was concentrated, dissolved in water/MeCN (3.5:1, 3 mL), and filtered through a 0.2 μm syringe filter. Purification was performed by preparative reverse-phase HPLC (Grace Alltima C18 5 μm column, 22×250 mm; gradient: 30 to 50% MeCN/0.1% TFA in water/0.1% TFA in 36 min). Product fractions were concentrated under reduced pressure resulting in a colorless solid (56 mg, 85% yield).

HPLC (Grace Alltima C18 5 μm column, 25×4.6 mm) indicated that the product compound L7-Boc was 100% pure (retention time 19.8 min; gradient: 5 to 50% MeCN/0.1% TFA in water/0.1% TFA in 20 min measured at a wavelength of 210 nm).

HRMS (ESI⁺) C₅₈H₁₀₂N₉O₁₂ [M+H]⁺ calc 1116.7642, found 1116.7774.

The obtained compound L7-Boc was dissolved in DCM (2 mL) and TFA (2 mL) was added. The mixture was stirred for 45 min at room temperature, followed by concentration under reduced pressure. The residue was dissolved in 10% MeOH/DCM (2 mL) and loaded on an ISOLUTE® SCX-2 column, pre-washed with DCM (10 mL). The column was washed with 10% MeOH/DCM (20 mL), and the product was eluted with 1 M methanolic ammonia in DCM (1:1). The combined product fractions were concentrated under reduced pressure and co-evaporated with MeOH several times to remove traces of ammonia affording a colorless solid (34 mg, 73% yield).

HPLC (Grace Alltima C18 5 μm column, 25×4.6 mm) indicated that the product was 99% pure (retention time 9.2 min; gradient: 20 to 100% MeCN/0.1% TFA in water/0.1% TFA in 20 min measured at a wavelength of 210 nm).

HRMS (ESI⁺) C₅₃H₉₄N₉O₁₀ [M+H]⁺ calc 1016.7118, found 1016.6976.

### 6.13.2. Synthesis of the Complex [AF-PEG₂-urea-pip-Pt(ethane-1,2-diamine)I]⁺ TFA⁻ (6m)

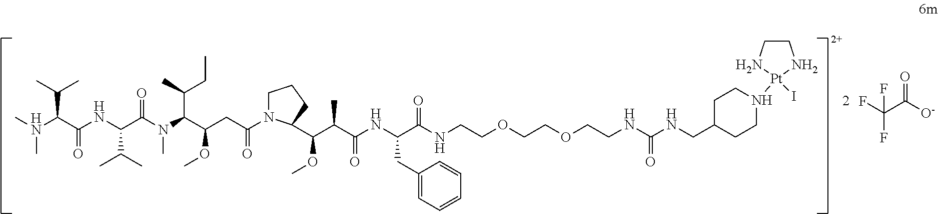

N-(3-Oxo-1-(piperidin-4-yl)-7,10-dioxa-2,4-diazadodecan-12-yl) AF amide (L7) (AF-pip; 15.0 mg, 15 μmot, 1.0 eq.) and Pt(ethane-1,2-diamine)I₂ (3a) (22.5 mg, 44 mot, 3.0 eq.) were dissolved in dry DMF (150 μL) under argon atmosphere. Diisopropyamine (7.71 μL, 44 μmol, 3.0 eq.) was added and the course of the reaction was followed by HPLC. The reaction mixture was stirred at 60° C. for 2 h. At this moment, the reaction mixture contained 100.0% product. The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (18.0 mg, 75.0% yield).

HRMS (ESI⁺) C₅₅H₁₀₂IN₁₁O₁₀¹⁹⁵Pt [M+H]²⁺ calc 699.3247, found 699.3198.

HPLC (Grace Alltima C18 5 μm column, 25×4.6 mm) indicated that the product was 98.9% pure (retention time 10.3 min; gradient: 20 to 100% MeCN/0.1% TFA in water/0.1% TFA in 20 min measured at a wavelength of 210 nm).

¹⁹⁵Pt-NMR (86 MHz, DMF-d₇): δ −3016

### 6.14. Synthesis of the Complex [AF-PEG₂-urea-pip-Pt((1R,2R)-cyclohexane-1,2-diamine)I]⁺ TFA⁻ (6n)

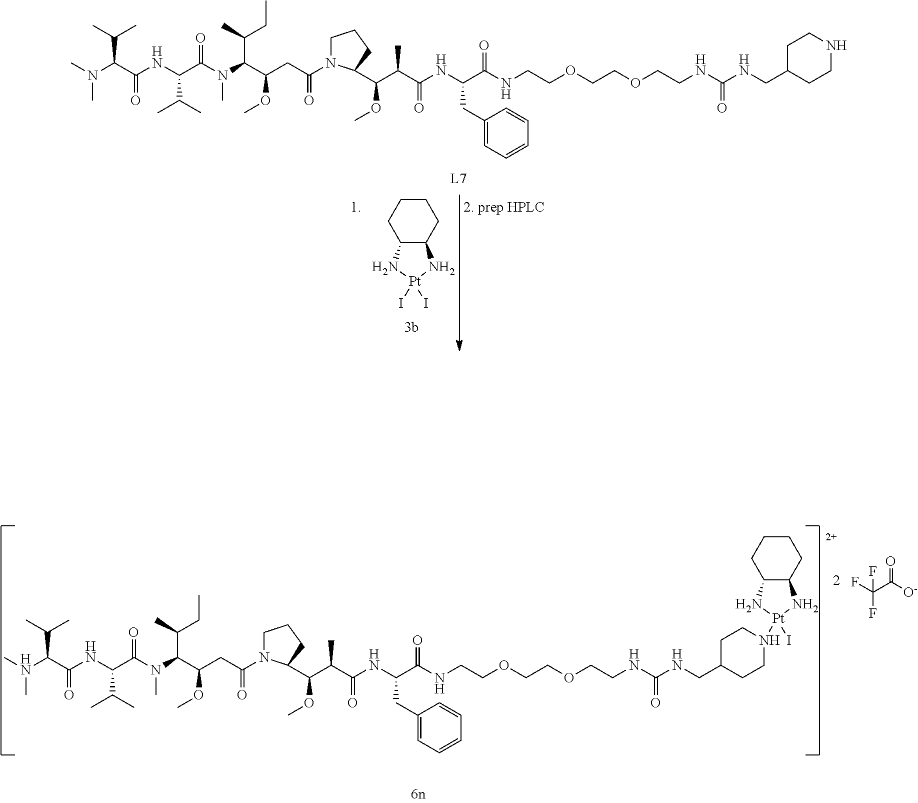

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(((1R,2R)-(−)-1,2-diaminocyclohexane))I₂ (3b) (24.8 mg, 44 mot, 3.0 eq.) were dissolved in dry DMF (150 μL) under argon atmosphere. Diisopropylethylamine (7.71 μL, 44 μmol, 3.0 eq.) was added and the course of the reaction was followed by HPLC. The reaction mixture was stirred at 60° C. for 4 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (15.6 mg, 59.0% yield).

HRMS (ESI⁺) C₅₉H₁₀₈IN₁₁O₁₀¹⁹⁵Pt [M+H]²⁺ calc 726.3481, found 726.3441.

HPLC (Grace Alltima C18 5 μm column, 25×4.6 mm) indicated that the product was 99.4% pure (retention time 11.0 min; gradient: 20 to 100% MeCN/0.1% TFA in water/0.1% TFA in 20 min measured at a wavelength of 210 nm).

### 6.15. Synthesis of the Complex [AF-PEG₂-urea-pip-Pt((1S,2S)-cyclohexane-1,2-diamine)I]⁺ TFA⁻ (6o)

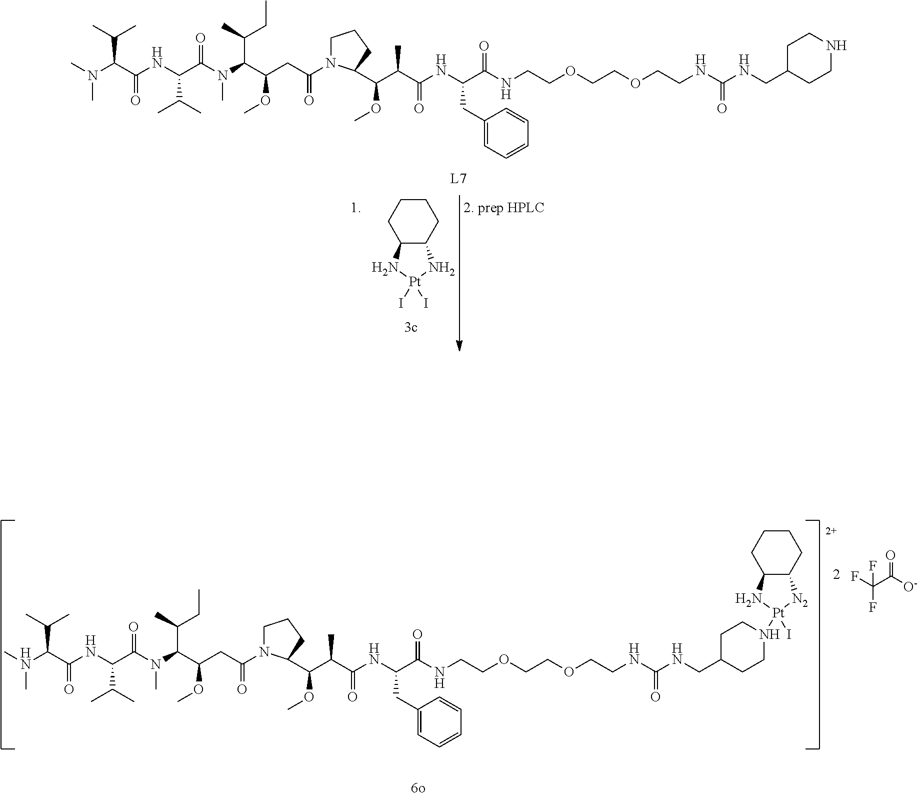

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(((1S,2S)-(−)-1,2-diaminocyclohexane))I₂ (3c) (26.1 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h. At this moment, the reaction mixture contained 100.0% product. The reaction mixture was diluted with a water/MeOH solution (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (18.4 mg, 71.1% yield).

HRMS (ESI⁺) C₅₉H₁₀₈IN₁₁O₁₀¹⁹⁵Pt [M+H]²⁺ calc 726.3481, found 726.3483.

HPLC (Grace Alltima C18 5 μm column, 25×4.6 mm) indicated that the product was 96.6% pure (retention time 11.3 min; gradient: 20 to 100% MeCN/0.1% TFA in water/0.1% TFA in 20 min measured at a wavelength of 210 nm).

### 6.16. Synthesis of the Complex [AF-PEG₂-urea-pip-Pt((1R,2S)-cyclohexane-1,2-diamine)I]⁺ TFA⁻ (6p)

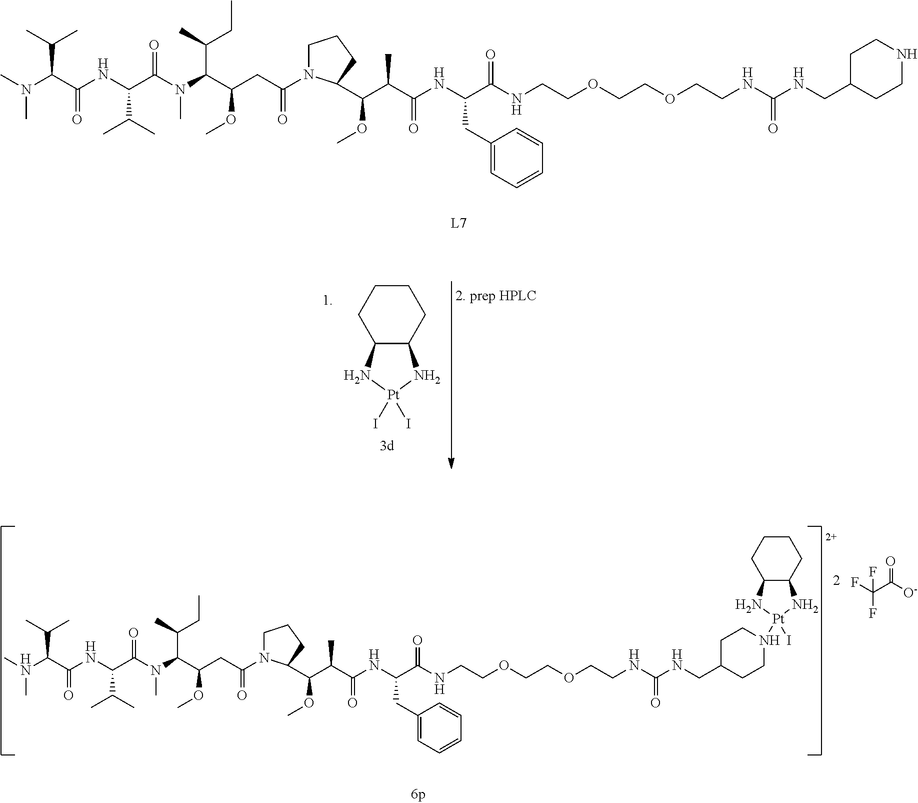

N-(3-Oxo-1-(piperidin-4-yl)-7,10-dioxa-2,4-diazadodecan-12-yl) AF amide (L7) (AF-pip; 20.0 mg, 20 μmol, 1.0 eq.) and Pt((1R,2S)-cyclohexane-1,2-diamine)I₂ (3d) (33.2 mg, 59 μmol, 3.0 eq.) were dissolved in dry DMF (150 μL) under argon atmosphere. Diisopropylethylamine (10.28 μL, 59 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% B in 40 min, A: 95/5 Water/MeOH+0.1% TFA and B: 5/95 Water/MeOH+0.1% TFA). Product fractions were concentrated under reduced pressure resulting in a colorless oil (22.1 mg, 66.9% yield).

HRMS (ESI⁺) C₅₉H₁₀₇IN₁₁O₁₀¹⁹⁵Pt [M+H]⁺ calc 1451.6893, found 1451.6847.

HPLC (Grace Alltima C18 5 μm column, 25×4.6 mm) indicated that the product was 98.6% pure (retention time 11.4 min; gradient: 20 to 100% MeCN/0.1% TFA in water/0.1% TFA in 20 min measured at a wavelength of 210 nm).

### 6.17. Synthesis of the Complex [AF-PEG₂-urea-pip-Pt(N¹,N²-dimethylethane-1,2-diamine)I]⁺ TFA⁻ (6q)

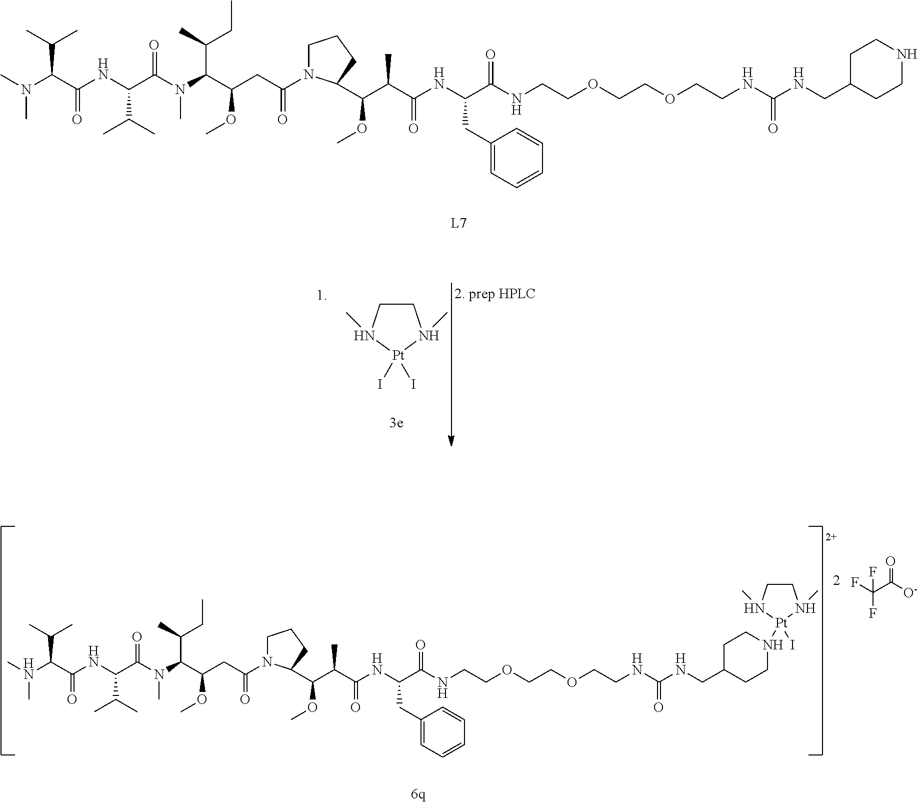

N-(3-Oxo-1-(piperidin-4-yl)-7,10-dioxa-2,4-diazadodecan-12-yl) AF amide (L7) (AF-pip; 20.0 mg, 20 μmol, 1.0 eq.) and Pt(N¹,N²-dimethylethane-1,2-diamine)I₂ (3e) (31.7 mg, 59 μmol, 3.0 eq.) were dissolved in dry DMF (150 μL) under argon atmosphere. Diisopropylethylamine (10.28 μL, 59 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% B in 40 min, A: 95/5 Water/MeOH+0.1% TFA and B: 5/95 Water/MeOH+0.1% TFA). Product fractions were concentrated under reduced pressure resulting in a colorless oil (27.6 mg, 84.8% yield).

HRMS (ESI⁺) C₅₇H₁₀₅IN₁₁O₁₀¹⁹⁵Pt [M]⁺ calc 1425.6736, found 1425.6701.

HPLC (Grace Alltima C18 5 μm column, 25×4.6 mm) indicated that the product was 97.0% pure (retention time 11.0 min; gradient: 20 to 100% MeCN/0.1% TFA in water/0.1% TFA in 20 min measured at a wavelength of 210 nm).

### 6.18. Synthesis of the Complex [AF-PEG₂-urea-pip-Pt(propane-1,3-diamine)I]⁺ TFA⁻ (6r)

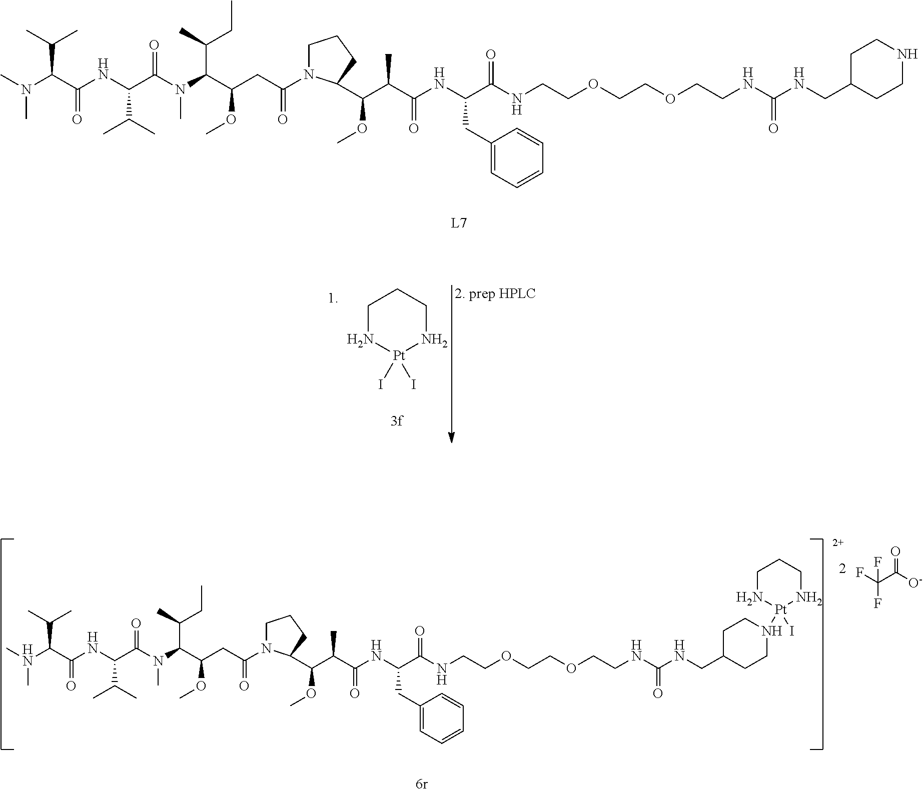

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and PtI₂(propane-1,3-diamine) (31) (24.7 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (15.4 mg, 59.6% yield).

HRMS (ESI⁺) C₅₆H₁₀₄IN₁₁O₁₀¹⁹⁵Pt [M+H]²⁺ calc 706.3325, found 706.3344.

HPLC (Grace Alltima C18 5 μm column, 25×4.6 mm) indicated that the product was 92.0% pure (retention time 10.5 min; gradient: 20 to 100% MeCN/0.1% TFA in water/0.1% TFA in 20 min measured at a wavelength of 210 nm).

### 6.19. Synthesis of the Complex [AF-PEG₂-urea-pip-Pt(1,3-diaminopropan-2-ol)I]⁺ TFA⁻ (6s)

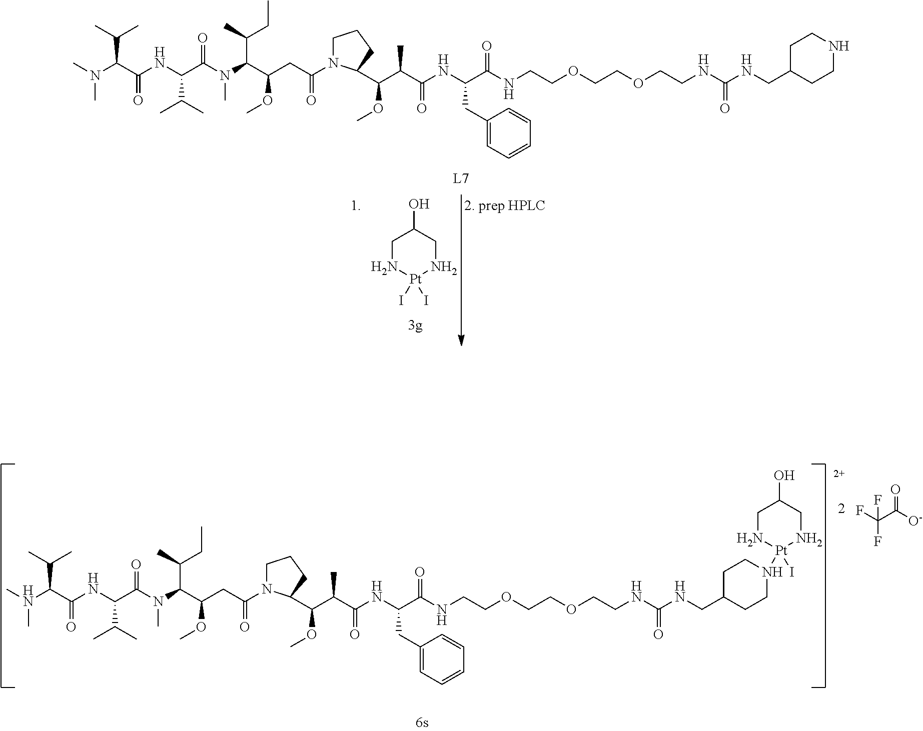

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(1,3-diaminopropan-2-ol)I₂ (3g) (23.9 mg, 44 μmol, 3.0 eq.) were dissolved in dry DMF (150 μL) under argon atmosphere. Diisopropyethylamine (7.71 μL, 44 μmol, 3.0 eq.) was added and the course of the reaction was followed by HPLC. The reaction mixture was stirred at 60° C. for 2 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (14.5 mg, 59.4% yield).

HRMS (ESI⁺) C₅₆H₁₀₄IN₁₁O₁₁¹⁹⁵Pt [M+H]²⁺ calc 714.3299, found 714.3254.

HPLC (Grace Alltima C18 5 μm column, 25×4.6 mm) indicated that the product was 94.4% pure (retention time 10.1 min; gradient: 20 to 100% MeCN/0.1% TFA in water/0.1% TFA in 20 min measured at a wavelength of 210 nm).

### 6.20. Synthesis of the Complex [AF-PEG₂-urea-pip-Pt(((1R,2R)-cyclobutane-1,2-diyl)dimethanamine)I]⁺ TFA⁻ (6t)

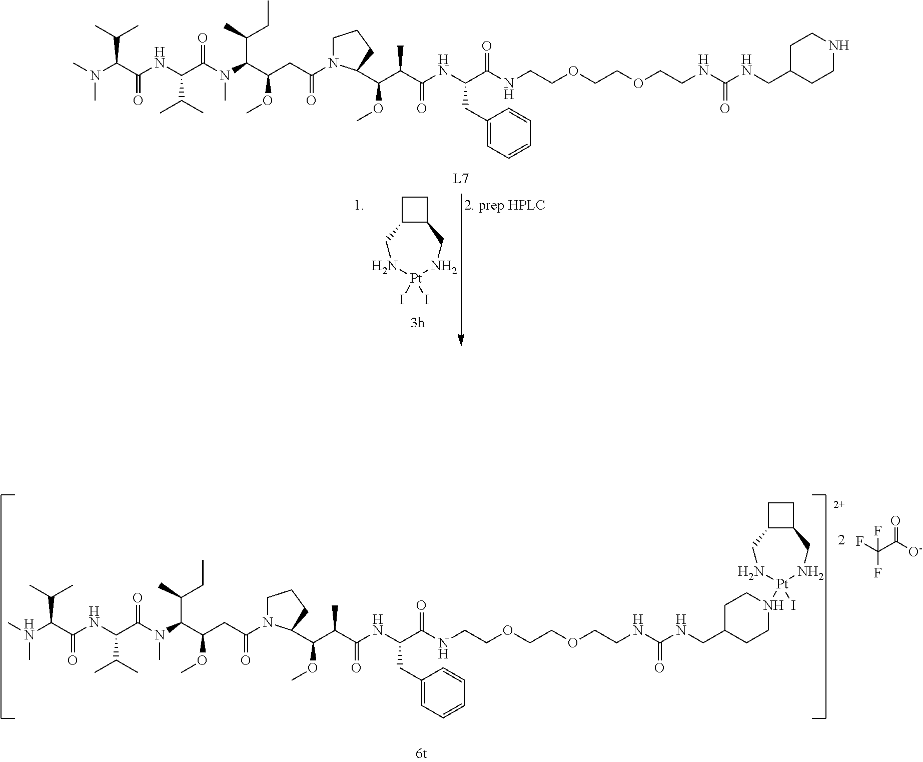

N-(3-Oxo-1-(piperidin-4-yl)-7,10-dioxa-2,4-diazadodecan-12-yl) AF amide (L7) (AF-pip; 15.0 mg, 15 μmot, 1.0 eq.) and Pt(((1R,2R)-cyclobutane-1,2-diyl)dimethanamine)I₂ (3h) (24.8 mg, 44 μmol, 3.0 eq.) were dissolved in dry DMF (150 μL) under argon atmosphere. Diisopropylethylamine (7.71 μL, 44 μmol, 3.0 eq.) was added and the course of the reaction was followed by HPLC. The reaction mixture was stirred at 60° C. for 2 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (8.6 mg, 34.7% yield).

HRMS (ESI⁺) C₅₉H₁₀₈IN₁₁O₁₁¹⁹⁵Pt [M+H]²⁺ calc 726.3481, found 726.3444.

HPLC (Grace Alltima C18 5 μm column, 25×4.6 mm) indicated that the product was 98.7% pure (retention time 11.6 min; gradient: 20 to 100% MeCN/0.1% TFA in water/0.1% TFA in 20 min measured at a wavelength of 210 nm).

### 6.21. Synthesis of the Complex [AF-PEG₂-urea-pip-Pt((3R,4R,5S,6R)-3,4-diamino-6-(hydroxymethyl)tetrahydro-2H-pyran-2,5-diol)I]⁺ TFA⁻ (6u)

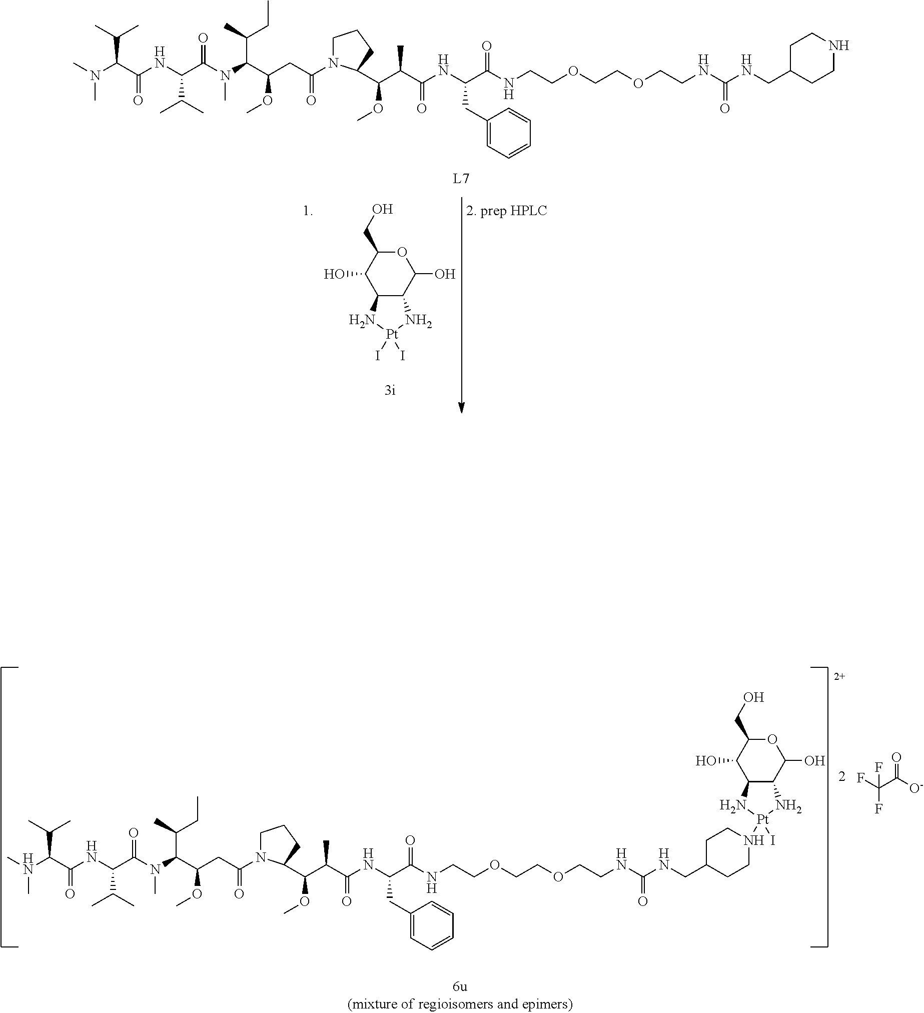

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt((3R,4R,5S,6R)-3,4-diamino-6-(hydroxymethyl)tetrahydro-2H-pyran-2,5-diol)I₂ (3i) (27.8 mg, 44 μmol, 3.0 eq.) were dissolved in dry DMF (150 μL) under argon atmosphere. N,N-Diisopropylethylamine (7.71 μL, 44 μmol, 3.0 eq.) was added and the course of the reaction was followed by HPLC. The reaction mixture was stirred at 60° C. for 3.5 h. At this moment, the reaction mixture contained 63.7% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (10.5 mg, 40.8% yield).

HRMS (ESI⁺) C₅₉₉H₁₀₈IN₁₁O₁₄¹⁹⁵Pt [M+H]⁺ calc 758.3379, found 758.3327.

HPLC (Grace Alltima C18 5 μm column, 25×4.6 mm) indicated that the product was 98.9% pure (note: the product was obtained as a mixture of regioisomers and epimers, observed as a broad peak; retention time 9.5 min; gradient: 20 to 100% MeCN/0.1% TFA in water/0.1% TFA in 20 min measured at a wavelength of 210 nm).

### 6.22. Synthesis of the Complex [AF-PEG₂-urea-pip-Pt((4aR,6R,7R,8R,8aS)-6-methoxy-2-phenylhexahydropyrano[3,2-d][1,3]dioxine-7,8-diamine)I]⁺ TFA⁻ (6v)

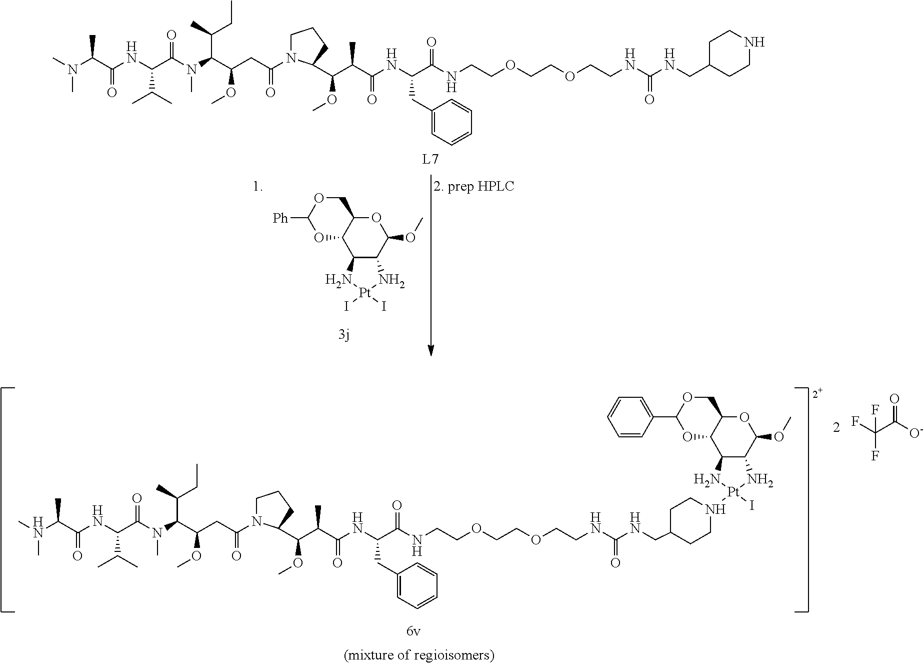

N-(3-Oxo-1-(piperidin-4-yl)-7,10-dioxa-2,4-diazadodecan-12-yl) AF amide (L7) (AF-pip; 16.0 mg, 16 μmot, 1.0 eq.) and Pt((4aR,6R,7R,8R,8aS)-6-methoxy-2-phenylhexahydropyrano[3,2-d][1,3]dioxine-7,8-diamine)I₂ (3j) (34.4 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (21.6 mg, 74.3% yield).

HRMS (ESI⁺) C₆₇H₁₁₄IN₁₁O₁₄¹⁹⁵Pt [M+H]²⁺ calc 809.3614, found 809.3633.

HPLC (Grace Alltima C18 5 μm column, 25×4.6 mm) indicated that the product was 96.3% pure (note: the product was obtained as a mixture of regioisomers, so that two peaks were observed; retention times 12.8 min and 13.2 min; gradient: 20 to 100% MeCN/0.1% TFA in water/0.1% TFA in 20 min measured at a wavelength of 210 nm).

### 6.23. Synthesis of the Complex [AF-PEG₂-urea-pip-Pt(2-((2-aminoethyl)amino)ethan-1-ol)I]⁺TFA⁻ (6w)

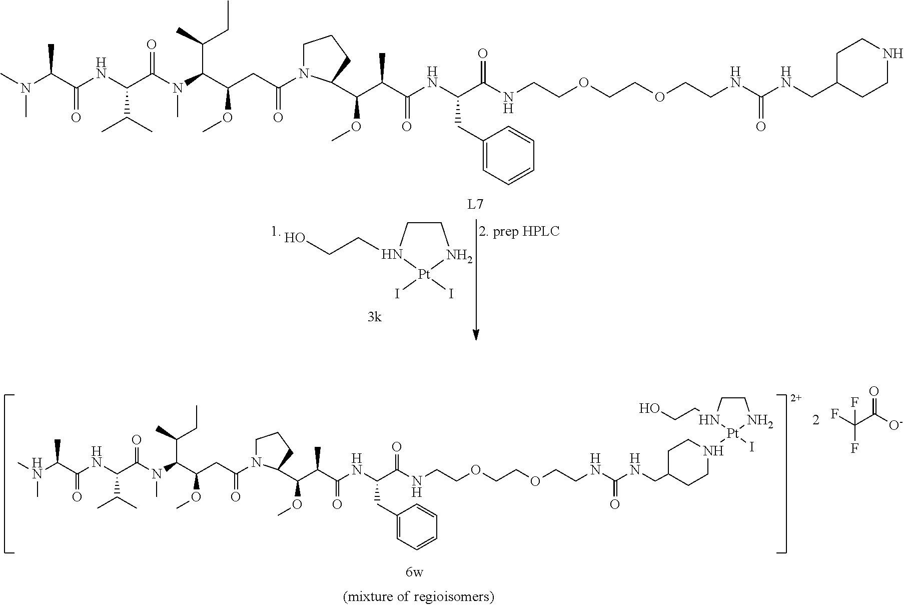

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(2-((2-aminoethyl)amino)ethan-1-ol)I₂ (3k) (26.1 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (17.4 mg, 66.2% yield).

HRMS (ESI⁺) C₅₇H₁₀₆IN₁₁O₁₁¹⁹⁵Pt [M+H]²⁺ calc 721.3377, found 721.3379.

HPLC (Grace Alltima C18 5 μm column, 25×4.6 mm) indicated that the product was 98.8% pure (note: the product was obtained as a mixture of presumably (regio)isomers, so that three peaks were observed; retention times 9.0 min, 10.1 min, and 10.4 min; gradient: 20 to 100% MeCN/0.1% TFA in water/0.1% TFA in 20 min measured at a wavelength of 210 nm).

### 6.24. Synthesis of the Complex [AF-PEG₂-urea-pip-Pt(2,2′-(ethane-1,2-diylbis(azanediyl))bis(ethan-1-ol))I]⁺ TFA⁻ (6x)

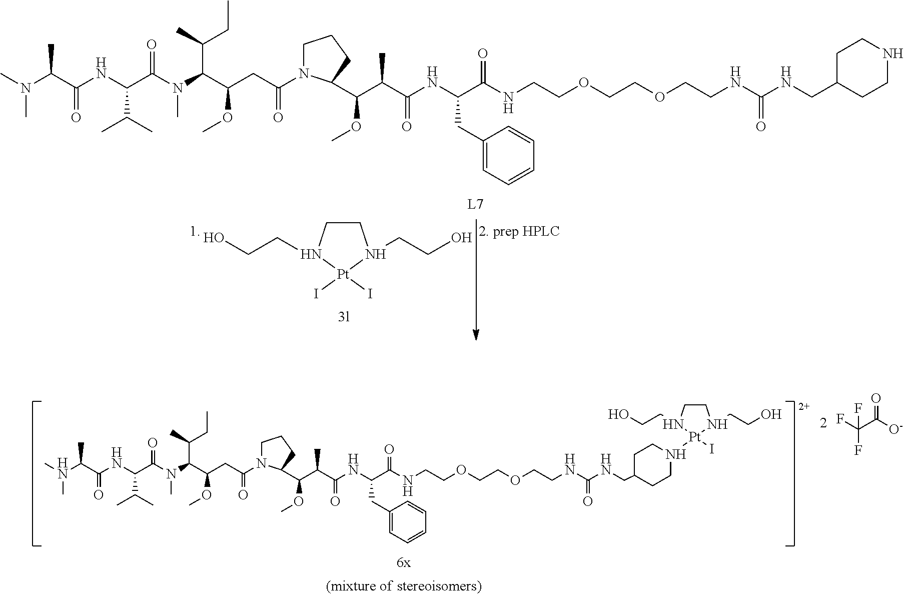

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(2,2′-(ethane-1,2-diylbis(azanediyl))bis(ethan-1-ol))I₂ (31) (28.2 mg, 47 μmol, 3.0 eq.) were dissolved in dry DMF (150 μL) under argon atmosphere. Diisopropylethylamine (8.23 μL, 47 μmot, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (10.5 mg, 38.9% yield).

HRMS (ESI⁺) C₅₉H₁₁₀IN₁₁O₁₂¹⁹⁵Pt [M+H]²⁺ calc 743.3508, found 743.3528.

HPLC (Grace Alltima C18 5 μm column, 25×4.6 mm) indicated that the product was 93.7% pure (note: the product was obtained as a mixture of presumably stereoisomers, so that two peaks were observed; retention times 9.0 min and 10.2 min; gradient: 20 to 100% MeCN/0.1% TFA in water/0.1% TFA in 20 min measured at a wavelength of 210 nm).

### 6.25. Synthesis and Analytical Characterization of the complex [AF-pip-Pt(ethane-1,2-diamine)I]⁺ TFA⁻ (6y)

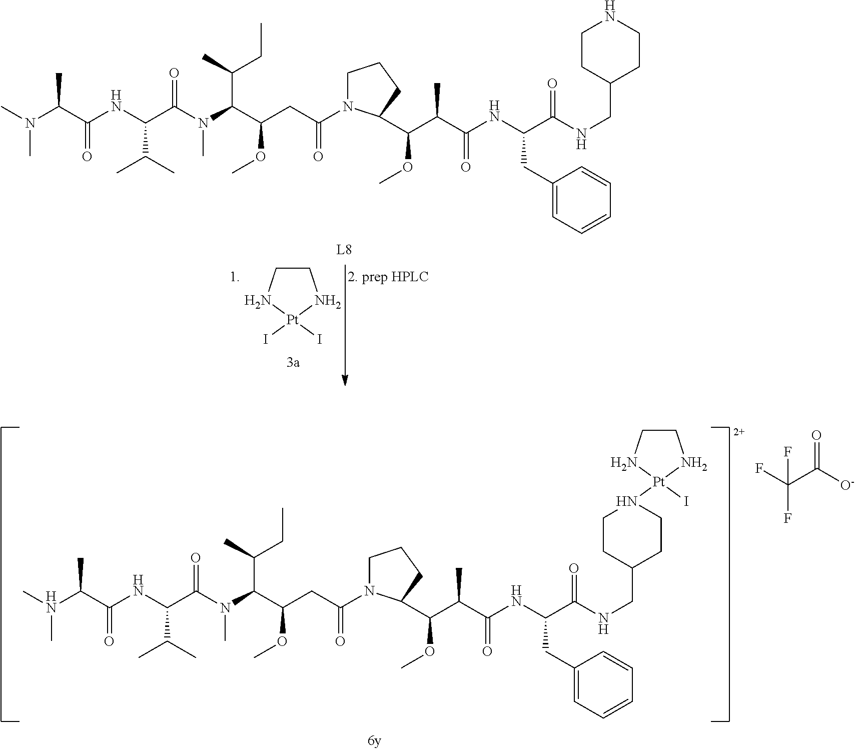

### 6.25.1. Synthesis of the Ligand AF-pip (L8)

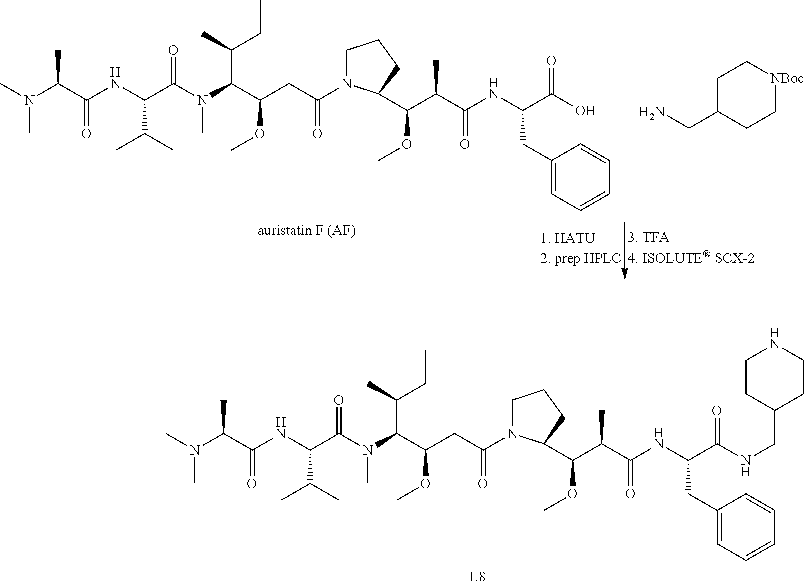 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C)N(C)C)C(C)C ; CC(C)(C)OC(=O)N1CCC(CN)CC1 ; CC[C@H](C)[C@@H]([C@@H](CC(=O)N1CCC[C@H]1[C@H](OC)[C@@H](C)C(=O)N[C@@H](Cc1ccccc1)C(=O)NCC1CCNCC1)OC)N(C)C(=O)[C@@H](NC(=O)[C@H](C)N(C)C)C(C)C]

Auristatin F (AF) (30.0 mg, 40 μmol, 1.0 eq.), dissolved in DMF (1.00 mL), was added to tert-butyl 4-(aminomethyl)piperidine-1-carboxylate (22.9 mg, 60 μmol, 1.5 eq). HATU (12.9 mg, 60 μmol, 1.5 eq.) and DIPEA (13.96 μL, 101 μmol, 2.5 eq.) were subsequently added and the mixture was stirred for 1 h in an ice bath. The reaction mixture was concentrated, dissolved in water/MeCN (3.5:1, 3 mL), and filtered through a 0.2 μm syringe filter. Purification was performed by preparative reverse-phase HPLC (Grace Alltima C18 5 μm column, 22×250 mm; gradient: 20 to 100% MeCN/0.1% TFA in water/0.1% TFA in 36 min). Product fractions were concentrated resulting in a colorless solid (44.5 mg, quant.).

HPLC (Grace Alltima C18 5 μm column, 25×4.6 mm) indicated that the product compound L8-Boc was 100.0% pure (95.9% compound L8-Boc: retention time 14.9 min and 4.1% Boc-deprotected compound compound L8: retention time 9.3 min; gradient: 20 to 100% MeCN/0.1% TFA in water/0.1% TFA in 20 min measured at a wavelength of 210 nm).

The obtained compound L8-Boc was dissolved in DCM (2 mL) and TFA (2 mL) was added. The mixture was stirred for 45 min at room temperature, followed by concentration under reduced pressure. The residue was dissolved in 10% MeOH/DCM (2 mL) and loaded on an ISOLUTE® SCX-2 column, pre-washed with DCM (10 mL). The column was washed with 10% MeOH/DCM (20 mL), and the product was eluted with 1 M methanolic ammonia in DCM (1:1). The combined product fractions were concentrated and co-evaporated with MeOH several times to remove traces of ammonia affording a colorless solid (22.7 mg, 63.0% yield).

HPLC (Grace Alltima C18 5 μm column, 25×4.6 mm) indicated that the product was 100% pure (retention time 9.3 min; gradient: 20 to 100% MeCN/0.1% TFA in water/0.1% TFA in 20 min measured at a wavelength of 210 nm).

HRMS (ESI⁺) C₄₆H₈₁N₇O₇ [M+2H]²⁺ calc 421.8093, found 421.8071.

### 6.25.2. Synthesis of the Complex [AF-pip-Pt(ethane-1,2-diamine)I]⁺ TFA⁻ (6y)

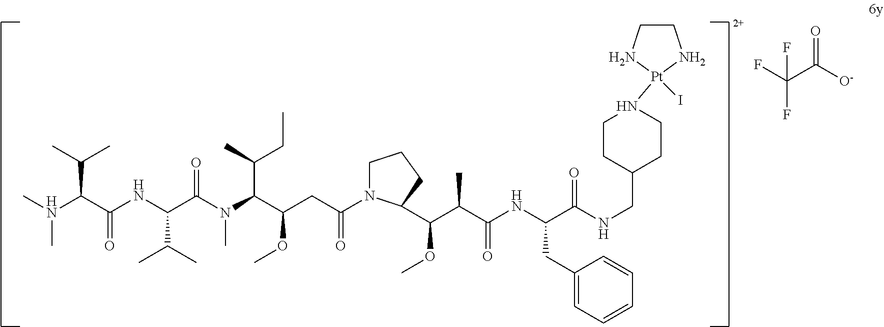

Auristatin F piperidinyl amide (L8) (AF-pip; 15.0 mg, 18 μmol, 1.0 eq.) and Pt(ethane-1,2-diamine)I₂ (3a) (27.2 mg, 53 μmol, 3.0 eq.) were dissolved in dry DMF (150 μL) under argon atmosphere. N,N-Diisopropylethylamine (9.33 μL, 53 μmol, 3.0 eq.) was added and the course of the reaction was followed by HPLC. The reaction mixture was stirred at 60° C. for 3.5 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated resulting in a colorless oil (15.3 mg, 59.1% yield).

HRMS (ESI⁺) C₄₈H₈₈IN₉O₇¹⁹⁵Pt [M+H]²⁺ calc 612.2744, found 612.2681.

HPLC (Grace Alltima C18 5 μm column, 25×4.6 mm) indicated that the product was 97.5% pure (retention time 10.5 min; gradient: 20 to 100% MeCN/0.1% TFA in water/0.1% TFA in 20 min measured at a wavelength of 210 nm).

### 6.26. Synthesis and Analytical Characterization of the complex [N-(14-azido-3,6,9,12-tetraoxatetradecyl)-3-(pyridin-4-yl)propanamide-Pt(ethane-1,2-diamine)I]⁺ TFA⁻ (6z)

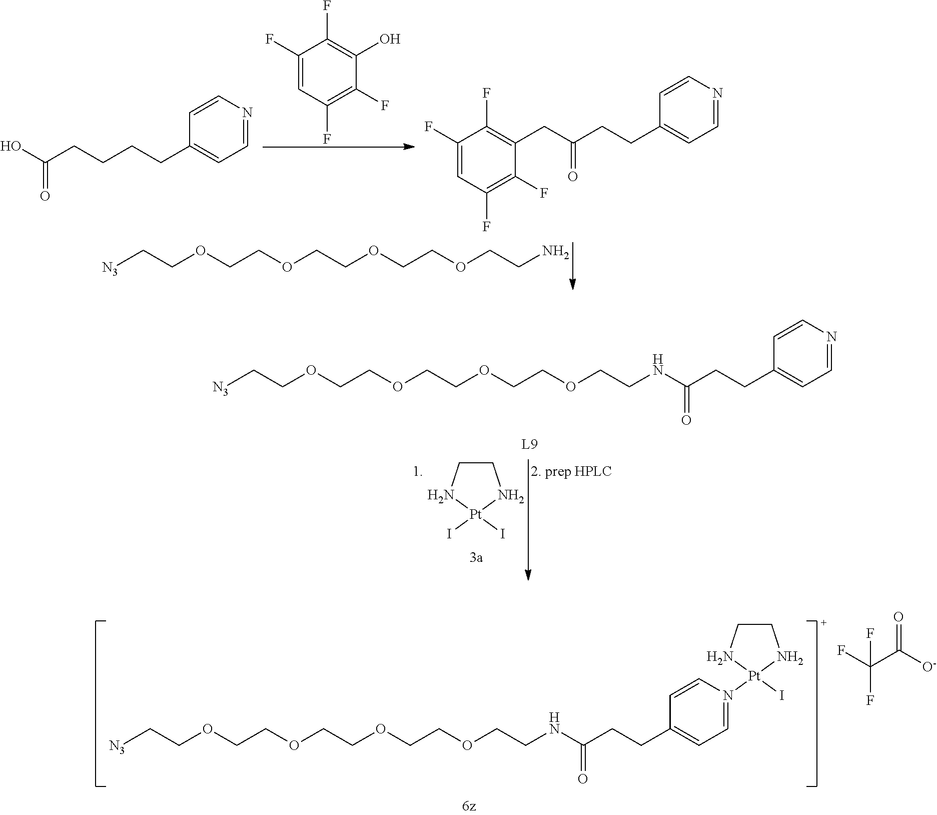

### 6.26.1. Synthesis of 2,3,5,6-tetrafluorophenyl 3-(pyridin-4-yl)propanoate

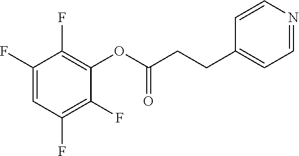 [SMILES: O=C(CCc1ccncc1)Oc1c(F)c(F)cc(F)c1F]

To a solution of 2,3,5,6-tetrafluorophenol (576 mg, 3.47 mmol, 1.1 eq.) in DCM (25 mL) was added 3-(pyridin-4-yl)propanoic acid (477 mg, 3.16 mmol, 1.0 eq.). The reaction mixture was stirred for 5 min at room temperature and EDC (726 mg, 3.79 mmol, 1.2 eq.) was added at room temperature. The resulting suspension was stirred for 60 h at room temperature. The reaction mixture was diluted with DCM (20 mL) and the mixture was washed with an aqueous 0.1 M HCl solution (prepared from 22.5 mL water and 2.5 mL 1 M HCl). The organic phase was subsequently washed with sat. NaHCO₃ solution and brine, dried with Na₂SO₄, and evaporated to dryness to obtain the crude product as a colorless solid (317 mg, 33.6% yield).

HPLC (Grace Alltima C18 5 μm column, 25×4.6 mm) indicated that the product was 96.5% pure (retention time 10.9 min; gradient: 20 to 100% MeCN/0.1% TFA in water/0.1% TFA in 20 min measured at a wavelength of 210 nm).

¹H NMR (400 MHz, DMSO-d₆): δ 8.54-8.39 (m, 2H), 8.00-7.80 (m, 1H), 7.39-7.25 (m, 2H), 3.24-3.15 (m, 2H), 3.06-2.94 (m, 2H).

### 6.26.2. Synthesis of the Ligand N-(14-azido-3,6,9,12-tetraoxatetradecyl)-3-(pyridin-4-yl)propanamide (N₃-PEG₄-py, L9)

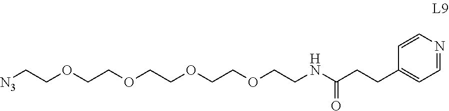 [SMILES: [N-]=[N+]=NCCOCCOCCOCCOCCNC(=O)CCc1ccncc1]

14-Azido-3,6,9,12-tetraoxatetradecan-1-amine (47.3 μL, 201 mot, 1.0 eq.) and 2,3,5,6-tetrafluorophenyl 3-(pyridin-4-yl)propanoate (60 mg, 201 μmol, 1.0 eq.) were dissolved in dry MeCN (2 mL) under argon atmosphere. This mixture was stirred for 2.5 h (the reaction progress was monitored by TLC using cyclohexane/EtOAc 1:2 and ⁱPrOH/NH_{3(aq.)}=10:1 as eluents). Then, TEA (27.9 μL, 201 μmol, 1.0 eq.) was added and the mixture was stirred for 20 h. After that, solvents were removed under reduced pressure to afford a colorless oily residue (119 mg) which was subsequently purified by column chromatography (step wise gradient using DCM/MeOH/NH_{3(aq.)}=100:5:1-100:7.5:1-100:10:1 as an eluent). The product containing fraction was evaporated under reduced pressure to afford a colorless oil (66 mg, 83% yield).

HRMS (ESI+) C₁₈H₃₀N₅O₅ [M+H]⁺ calc 396.2241, found 396.2260.

### 6.26.3. Synthesis of the Complex [N-(14-azido-3,6,9,12-tetraoxatetradecyl)-3-(pyridin-4-yl)propanamide-Pt(ethane-1,2-diamine)I]⁺ TFA⁻ (6z)

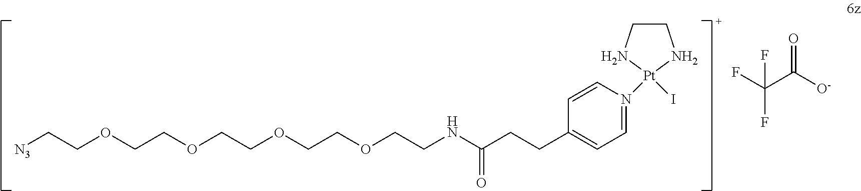

N-(14-Azido-3,6,9,12-tetraoxatetradecyl)-3-(pyridin-4-yl)propanamide (L9) (N³-PEG₄-py; 22.5 mg, 57 μmol, 1.0 eq.) and Pt(ethane-1,2-diamine)I₂ (3a) (87.0 mg, 171 μmol, 3.0 eq.) were dissolved in dry DMF (500 μL) under argon atmosphere. Diisopropylethylamine (29.7 μL, 171 μmol, 3.0 eq.) was added, the reaction mixture was stirred at 40° C. for 24 h, and the course of the reaction was followed by HPLC. The reaction mixture was diluted with a 10 mM NaI/MeOH mixture (4:1, 2.5 mL) and filtered through a 0.2 μm syringe filter. Purification was performed by preparative reverse-phase HPLC (Grace Alltima C18 5 μm column, 22×250 mm; gradient: 20 to 75% MeOH/0.1% TFA in water/0.1% TFA in 36 min). Product fractions were concentrated under reduced pressure affording a colorless oil (36.8 mg, 72.6% yield).

HRMS (ESI⁺) C₂₀H₃₇IN₇O₅¹⁹⁵Pt [M]⁺ calc 777.1543, found 777.1540.

HPLC (Grace Alltima C18 5 μm column, 25×4.6 mm) indicated that the product was 95.7% pure (retention time 16.6 min; gradient: 5 to 50% MeCN/0.1% TFA in water/0.1% TFA in 20 min measured at a wavelength of 210 nm).

### 6.27. Synthesis and Analytical Characterization of the complex [N¹,N³-bis(14-azido-3,6,9,12-tetraoxatetradecyl)-N⁵-(pyridin-4-ylmethyl)benzene-1,3,5-tricarboxamide-Pt(ethane-1,2-diamine)I]⁺ TFA⁻ (6aa)

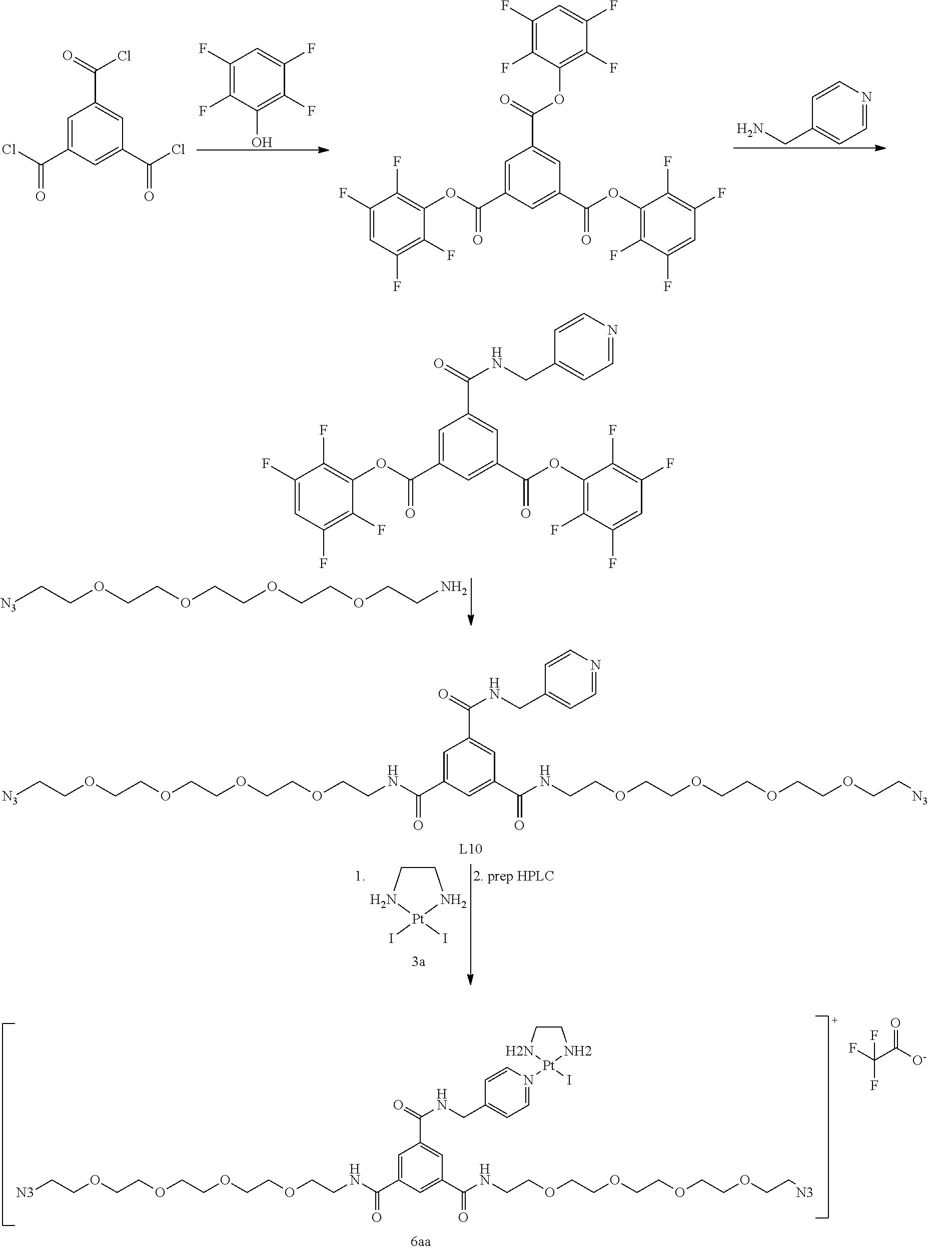

### 6.27.1. Synthesis of tris(2,3,5,6-tetrafluorophenyl)benzene-1,3,5-tricarboxylate

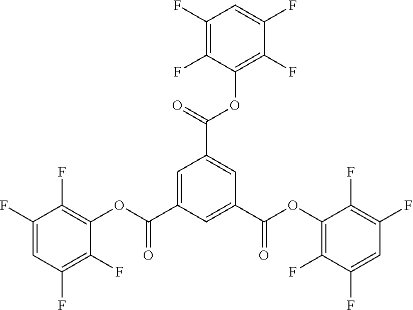 [SMILES: O=C(Oc1c(F)c(F)cc(F)c1F)c1cc(C(=O)Oc2c(F)c(F)cc(F)c2F)cc(C(=O)Oc2c(F)c(F)cc(F)c2F)c1]

Under argon atmosphere, DIPEA (13.9 mL, 80 mmol, 4.0 eq.) and 2,3,5,6-tetrafluorophenol (10.3 g, 60.4 mmol, 3.0 eq.) were dissolved in dry DCM (100 mL) and were subsequently added dropwise over 2.5 h to a rigorously stirred solution of benzene-1,3,5-tricarbonyl trichloride (3.57 mL, 20.0 mmol, 1.0 eq.) in dry DCM (150 mL) at 0° C. After addition, the mixture was stirred for 40 min and was allowed to warm to 6° C., after which it was gradually heated to the ambient temperature and stirred for another 1 h. Then, the reaction mixture was washed with 1 M HCl (320 mL) and with 1 M NaOH (320 mL). The alkaline aqueous layer was extracted with DCM (50 mL) and the combined organic layers were washed with brine (100 mL). The organic phase was dried with Na₂SO₄, filtered, and evaporated under reduced pressure. After removal of solvents, a pale brown solid (12.1 g, 93% yield) was obtained.

¹H NMR (400 MHz, CDCl₃): δ 9.30 (s, 3H), 7.17-7.05 (m, 3H)

### 6.27.2. Synthesis of bis(2,3,5,6-tetrafluorophenyl) 5-((pyridin-4-ylmethyl)carbamoyl)isophthalate

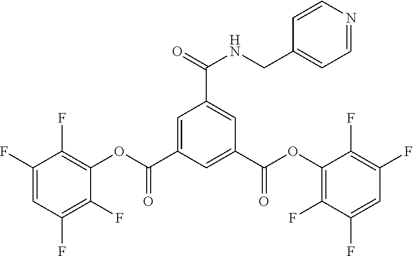 [SMILES: O=C(NCc1ccncc1)c1cc(C(=O)Oc2c(F)c(F)cc(F)c2F)cc(C(=O)Oc2c(F)c(F)cc(F)c2F)c1]

Tris(2,3,5,6-tetrafluorophenyl) benzene-1,3,5-tricarboxylate (5.00 g, 7.64 mmol, 3.0 eq.) was dissolved in DCM (100 mL). To this solution the mixture of pyridin-4-ylmethanamine (259 μL, 2.55 mmol, 1.0 eq.) and TEA (710 μL, 5.09 mmol, 2.0 eq.) in DCM (50 mL) was added dropwise over 140 min under vigorous stirring. Then, the mixture was stirred for another 1.5 h, after which TLC (DCM/MeOH/NH_{3(aq.)}=100:10:1 as an eluent) indicated a full consumption of pyridin-4-ylmethanamine. The solvents were removed under reduced pressure and the residue was suspended in cyclohexane/EtOAc (3:12, 15 mL), sonicated in ultrasound bath, filtered, and the filter cake was washed with cyclohexane/EtOAc (1:2, 4 mL). TLC revealed that the filter cake contained tris(2,3,5,6-tetrafluorophenyl) benzene-1,3,5-tricarboxylate starting material and the filtrate contained product along with this starting material. Therefore, the filtrate was evaporated under reduced pressure and the crude residue was suspended in cyclohexane/EtOAc (1.5:6, 7.5 mL), sonicated in ultrasound bath, filtered, and the filter cake was washed with cyclohexane/EtOAc (1:4, 3 mL). The yield of the recovered tris(2,3,5,6-tetrafluorophenyl) benzene-1,3,5-tricarboxylate was 1.67 g (33.3% of the applied amount). Finally, the filtrate was evaporated under reduced pressure, the residue was dissolved in cyclohexane/EtOAc (1:1, 12 mL) and purified by column chromatography (step wise gradient using cyclohexane/EtOAc 2:1→1:1 as an eluent). The collected product containing fractions were evaporated under reduced pressure, the residue was dissolved in DCM (100 mL). The obtained organic phase was washed with 1 M NaOH (40 mL), dried with Na₂SO₄, filtered, and evaporated affording a colorless solid (691 mg, 46% yield).

¹H NMR (400 MHz, DMSO-d₆): δ 9.77-9.70 (m, 1H), 9.09-9.05 (m, 2H), 8.97 (s, 1H), 8.54-8.50 (m, 2H), 8.12-7.97 (m, 2H), 7.38-7.34 (m, 2H), 4.58 (d, J=5.7 Hz, 2H).

### 6.27.3. Synthesis of the Ligand N¹,N³-bis(14-azido-3,6,9,12-tetraoxatetradecyl)-N⁵-(pyridin-4-ylmethyl)benzene-1,3,5-tricarboxamide (bis-N³-PEG₄-benzene-py, L10)

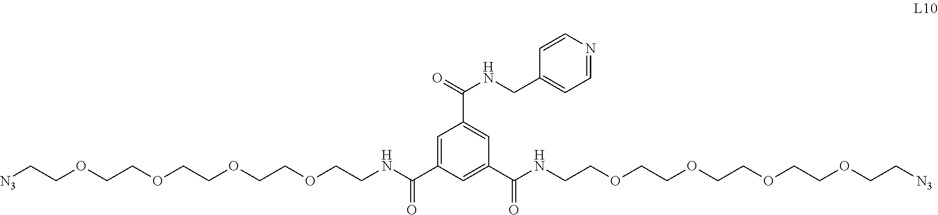 [SMILES: [N-]=[N+]=NCCOCCOCCOCCOCCNC(=O)c1cc(C(=O)NCCOCCOCCOCCOCCN=[N+]=[N-])cc(C(=O)NCc2ccncc2)c1]

Bis(2,3,5,6-tetrafluorophenyl) 5-((pyridin-4-ylmethyl)carbamoyl)isophthalate (88 mg, 0.15 mmol, 1.0 eq.) was dissolved in dry EtOAc/THF (5:2, 7 mL), followed by the addition of 14-azido-3,6,9,12-tetraoxatetradecan-1-amine (79 mg, 0.3 mmol, 2.0 eq.; dissolved in EtOAc (0.5 mL)), and TEA (61.7 μL, 0.44 mmol, 3.0 eq.). The resulting mixture was stirred under argon atmosphere at room temperature for 20 h. TLC (cyclohexane/EtOAc=1:2 and DCM/MeOH/NH_{3(aq.)}=100:10:1 as eluents) showed a full consumption of both bis(2,3,5,6-tetrafluorophenyl) 5-((pyridin-4-ylmethyl)carbamoyl)isophthalate and 14-azido-3,6,9,12-tetraoxatetradecan-1-amine. The solvent was removed under reduced pressure and the residue was purified by column chromatography (DCM:MeOH=100:1→to 100:2→100:3→100:5→100:7). After evaporation of solvents, the collected product containing fractions gave a pale orange oil (96 mg, 82% yield).

HRMS (ESI+) C₃₅H₅₃N₁₀O₁₁ [M+H]⁺ calc 789.3890, found 789.3868.

¹H NMR (400 MHz, CDCl₃): δ 8.58-8.51 (m, 2H), 8.47-8.43 (m, 2H), 8.39-8.36 (m, 1H), 8.08-8.00 (m, 1H), 7.57-7.50 (m, 2H), 7.35-7.30 (m, 2H), 4.65 (d, J=5.9 Hz, 2H), 3.71-3.51 (m, 36H), 3.34-3.27 (in, 4H).

### 6.27.4. Synthesis of the Complex N¹,N³-bis(14-azido-3,6,9,12-tetraoxatetradecyl)-N⁵-(pyridin-4-ylmethyl)benzene-1,3,5-tricarboxamide-Pt(ethane-1,2-diamine)I]⁺ TFA⁻ (6aa)

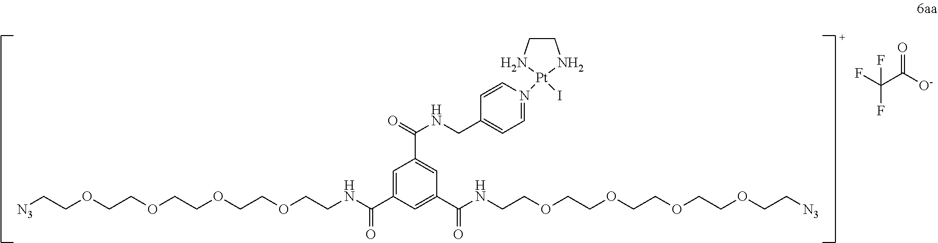

N¹,N³-Bis(14-azido-3,6,9,12-tetraoxatetradecyl)-N⁵-(pyridin-4-ylmethyl)benzene-1,3,5-tricarboxamide (L10) (bis-N³-PEG₄-benzene-py; 39.5 mg, 50 μmol, 1.0 eq.) and Pt(ethane-1,2-diamine)I₂ (3a) (25.4 mg, 50 μmol, 1.0 eq.) were dissolved in dry DMF (500 μL) under argon atmosphere resulting in a homogeneous yellow mixture. The reaction mixture was stirred at 50° C. for 19 h, and the course of the reaction was followed by HPLC. Then, additional Pt(ethane-1,2-diamine)I₂ (3a) (25.4 mg, 50 μmol, 1.0 eq.) was added to the reaction mixture. The reaction mixture was stirred at 50° C. for 24 h, and the course of the reaction was followed by HPLC. Thereafter, additional Pt(ethane-1,2-diamine)I₂ (3a) (25.4 mg, 50 μmol, 1.0 eq.) was added to the reaction mixture. The reaction mixture was stirred at 50° C. for 24 h, and the course of the reaction was followed by HPLC. At this moment, the reaction mixture contained 98.1% product.

The reaction mixture was diluted with water (10 mL) and filtered through a paper filter to remove precipitated excessive Pt(ethane-1,2-diamine)I₂ (3a). The filtrate was applied to a column containing RP-C18 (LiChroprep®, 15-25 μm; 500 mg, prewashed with MeOH (3 mL)). The run-out was discarded. The column was then washed subsequently with water/MeOH (9:1, 9 mL) and with water/MeOH (8:2, 5 mL). After that, the product was eluted with water/MeOH (2:8, 4 mL). HPLC analysis indicated that this fraction contained 99.6% product. This fraction was mixed with a NaI (13.2 mg) solution in water (1 mL). The mixture was further diluted with water (5 mL) and concentrated under reduced pressure. After been frozen, the mixture was lyophilized giving a yellow film (62.0 mg; corrected for the NaI content: 48.8 mg, 76.0% yield). The material was used to prepare a 5 mM solution in a 10 mM aqueous NaI solution; in this form the material was used and stored.

HRMS (ESI⁺) C₃₇H₆₀IN₁₂O₁₁¹⁹⁵Pt [M]⁺ calc 1170.3194, found 1170.3204.

HPLC (Grace Alltima C18 5 μm column, 25×4.6 mm) indicated that the product was 99.0% pure (retention time 11.2 min; gradient: 20 to 100% MeCN/0.1% TFA in water/0.1% TFA in 20 min measured at a wavelength of 223 nm).

Comparison of Conjugation Efficiencies Using Different Halido “Semi-Final products” (SFMs)

Without NaI in the Conjugation Mixture

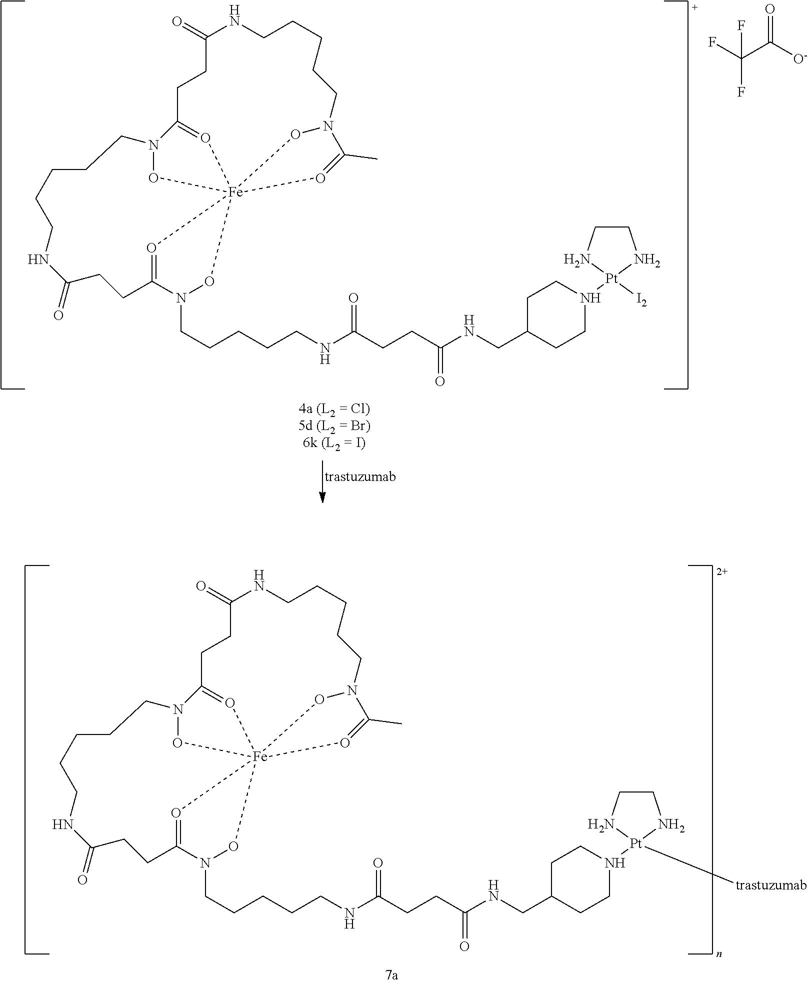

Trastuzumab (Herceptin®; 35.5 μL, 21 mg/mL, 1.0 eq.), rebuffered from the pharmacy storage buffer to PBS by spin filtration, was diluted with water (15 μL), 200 mM HEPES buffer (6.15 μL, pH 8.1), and [PtL₂((Fe)DFO-suc-pip)(ethane-1,2-diamine)]⁺ TFA⁻ (4a (L₂=Cl), 5d (L₂=Br) or 6k (L₂=I)) (5.0 μL, 5 mM in 20 mM NaCl (4a) or 5 mM in water (5d and 6k), 5.0 eq.) was added. The sample was incubated in a thermoshaker at 47° C. for 1 h, 2 h, 4 h, 6 h, and 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

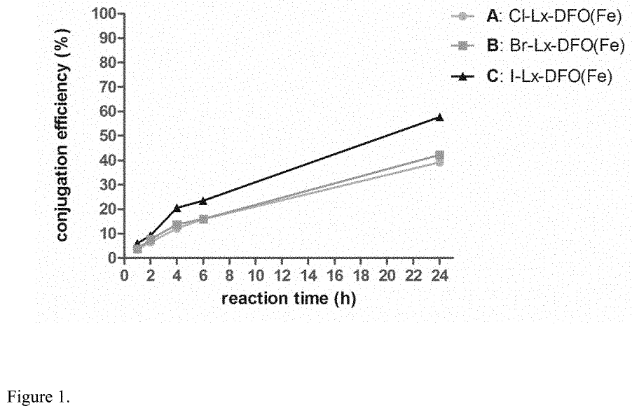

After 24 h conjugation time, the conjugation efficiencies were: 39% (A: 4a, L₂=Cl), 42% (B: 5d, L₂=Br), and 58% (C: 6k, L₂=I; FIG. 1).

With NaI in the Conjugation Mixture

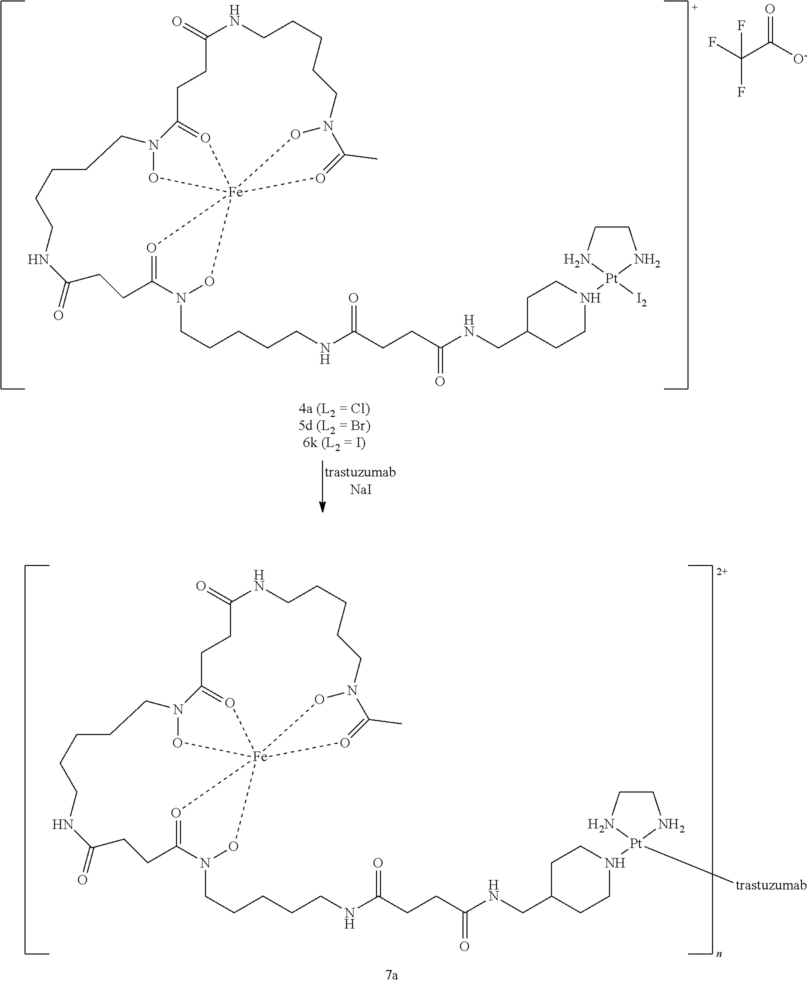

Trastuzumab (Herceptin®; 35.5 μL, 21 mg/mL, 1.0 eq.), rebuffered from the pharmacy storage buffer to PBS by spin filtration, was diluted with water (15 μL), 200 mM HEPES buffer (6.15 μL, pH 8.1) containing 100 mM NaI (the final concentration of NaI in the reaction mixture was 10 mM), and [PtL₂((Fe)DFO-suc-pip)(ethane-1,2-diamine)]⁺ TFA⁻ (4a (L₂=Cl), 5d (L₂=Br) or 6k (L₂=I)) (5.0 lμL, 5 mM in 20 mM NaCl (4a) or 5 mM in water (5d and 6k), 5.0 eq.) was added. The sample was incubated in a thermoshaker at 47° C. for 1 h, 2 h, 4 h, 6 h, and 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

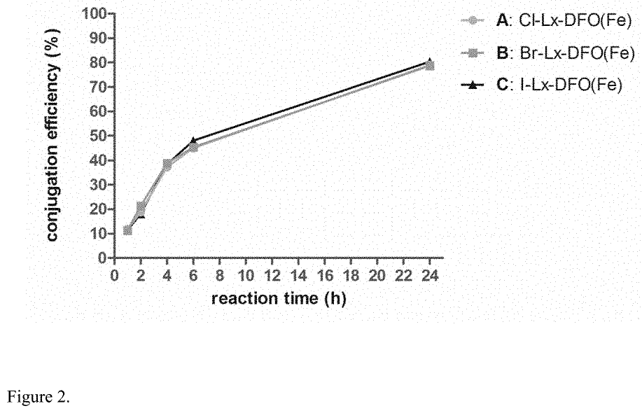

After 24 h conjugation time, the conjugation efficiencies were: 79% (A: 4a, L₂=Cl), 79% (B: 5d, L₂=Br), and 80% (C: 6k, L₂=I; FIG. 2).

Addition of the Corresponding Halide Salts

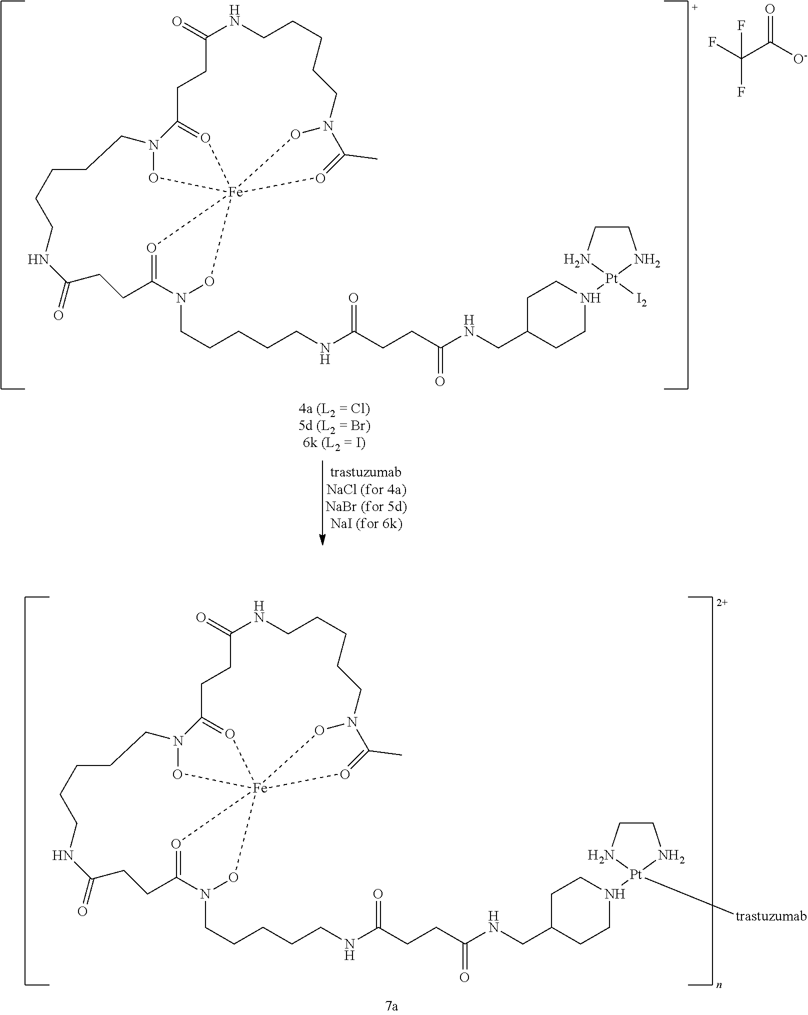

Trastuzumab (Herceptin®; 35.5 μL, 21 mg/mL, 1.0 eq.), rebuffered from the pharmacy storage buffer to 20 mM HEPES buffer (pH 8.1) by spin filtration, was diluted with water (15 μL), 200 mM HEPES buffer (6.15 μL, pH 8.1) containing 2000 mM NaCl (for 4a; pH 8.1; the final concentration of NaCl in the reaction mixture was 200 mM), 500 mM NaBr (for 5d; pH 8.1; the final concentration of NaBr in the reaction mixture was 50 mM) or 100 mM NaI (for 6k; pH 8.1; the final concentration of NaI in the reaction mixture was 10 mM), and [PtL₂((Fe)DFO-suc-pip)(ethane-1,2-diamine)]⁺ TFA⁻ (4a (L₂=Cl), 5d (L₂=Br) or 6k (L₂=I)) (5.0 μL, 5 mM in 20 mM NaCl (4a) or 5 mM in water (5d and 6k), 5.0 eq.) was added. The sample was incubated in a thermoshaker at 47° C. for 1 h, 2 h, 4 h, 6 h, and 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

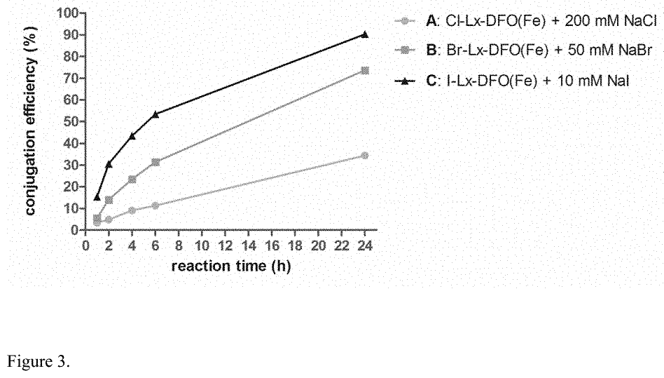

After 24 h conjugation time, the conjugation efficiencies were: 34% (A: 4a, L₂=Cl), 74% (B: 5d, L₂=Br), and 90% (C: 6k, L₂=I; FIG. 3).

Stabilization of Different Halido “Semi-Final Products” Under the Conjugation Conditions Using Excess of Corresponding Halide Salts; Determination of the Optimal Halide Salt Concentration to Prevent Hydrolysis of the “Semi-Final Products”

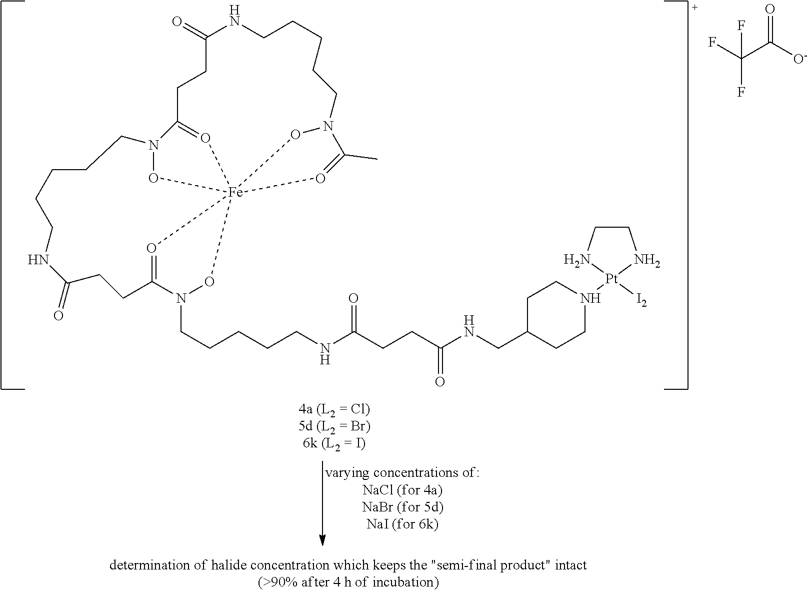

To a mixture of water (101 μL) and 200 mM HEPES buffer (12.3 μL, pH 8.1) containing different concentrations of halide salts (0, 100, 500, 1000, and 2000 mM NaCl (for 4a; pH 8.1; the final concentrations of NaCl in the reaction mixtures were 0, 10, 50, 100, and 200 mM, respectively) or 0, 100, and 500 mM NaBr (for 5d; pH 8.1; the final concentrations of NaBr in the reaction mixtures were 0, 10, and 50 mM, respectively) or 0 and 100 mM NaI (for 6k; pH 8.1; the final concentrations of NaI in the reaction mixtures were 0 and 10 mM, respectively), [PtL₂((Fe)DFO-suc-pip)(ethane-1,2-diamine)]⁺ TFA⁻ (4a (L₂=Cl), 5d (L₂=Br) or 6k (L₂=I)) (10.0 μL, 5 mM in 20 mM NaCl (4a) or 5 mM in water (5d and 6k), 5.0 eq.) was added. The samples were incubated in a thermoshaker at 47° C. for 1 h, 2 h, and 4 h, followed by the HPLC analysis at 430 nm.

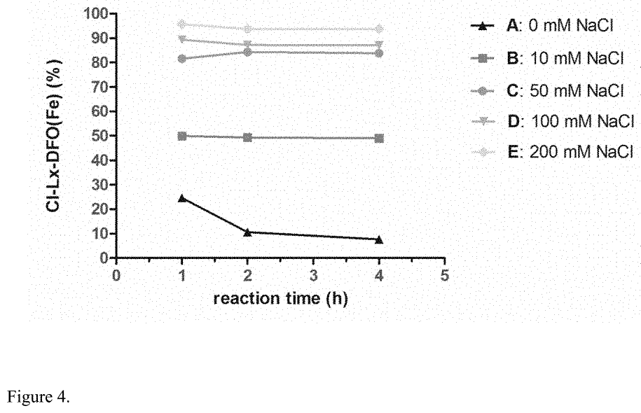

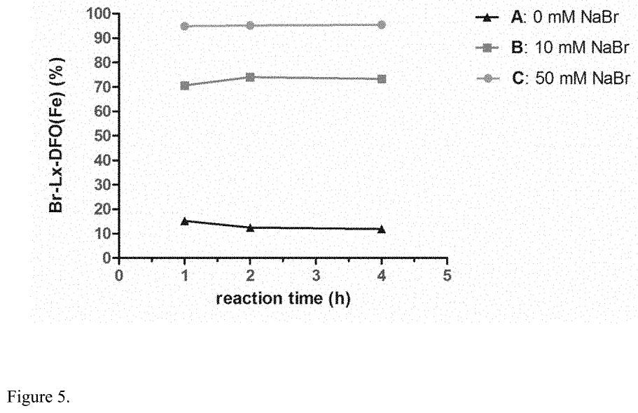

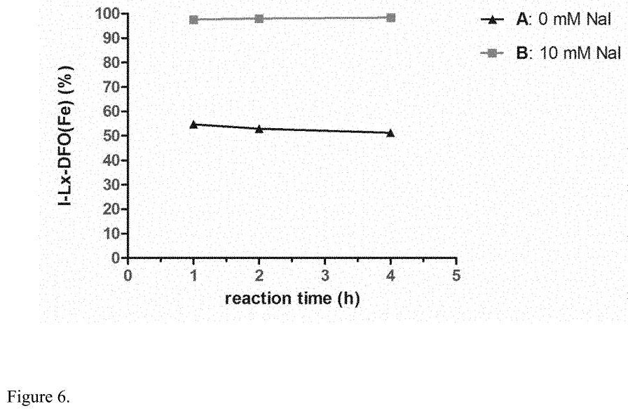

After 4 h incubation time, the concentrations of the “semi-final products” were determined as follows: 94% (E: 4a, L₂=Cl, [NaCl]=200 mM, FIG. 4), 95% (C: 5d, L₂=Br, [NaBr]=50 mM, FIG. 5), and 98% (C: 6k, L₂=I; [NaI]=10 mM, FIG. 6).

### Example 7: Examples of Trastuzumab-Lx Conjugates 7a-j

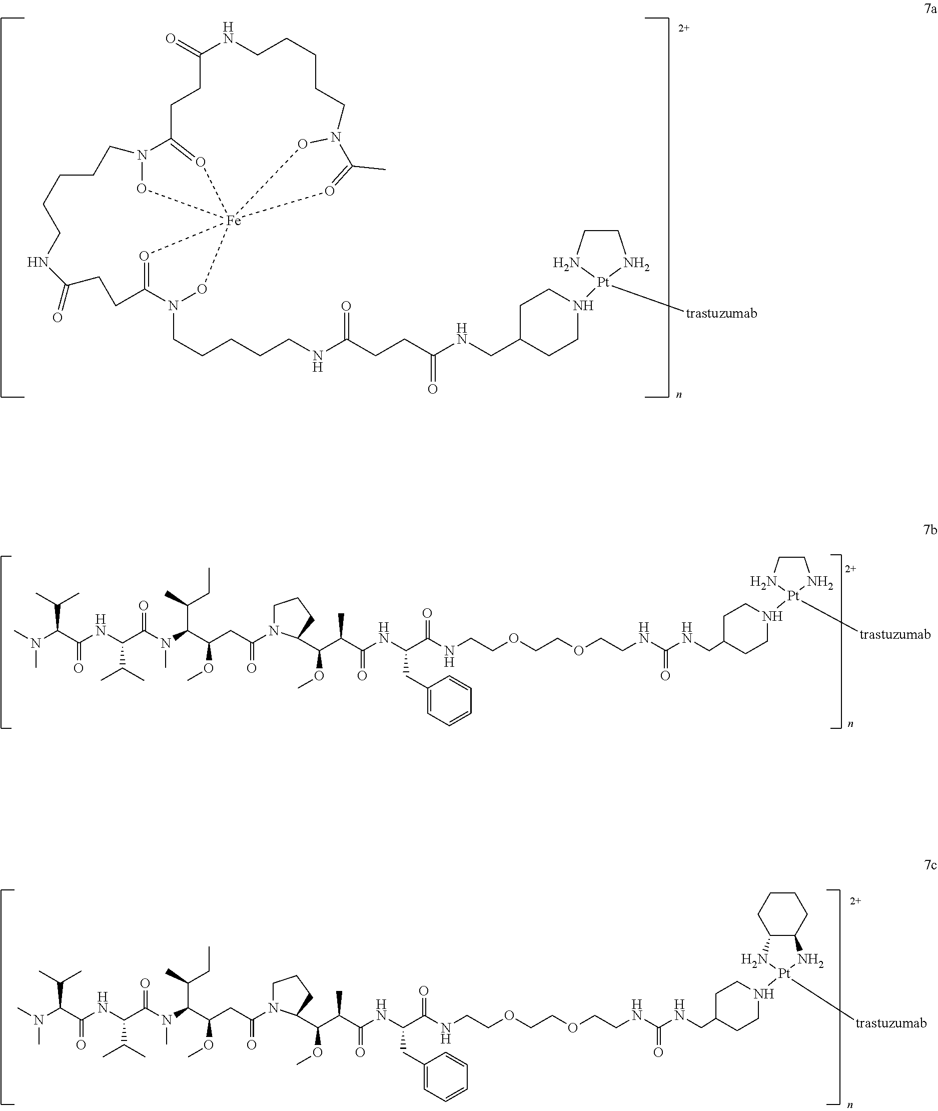

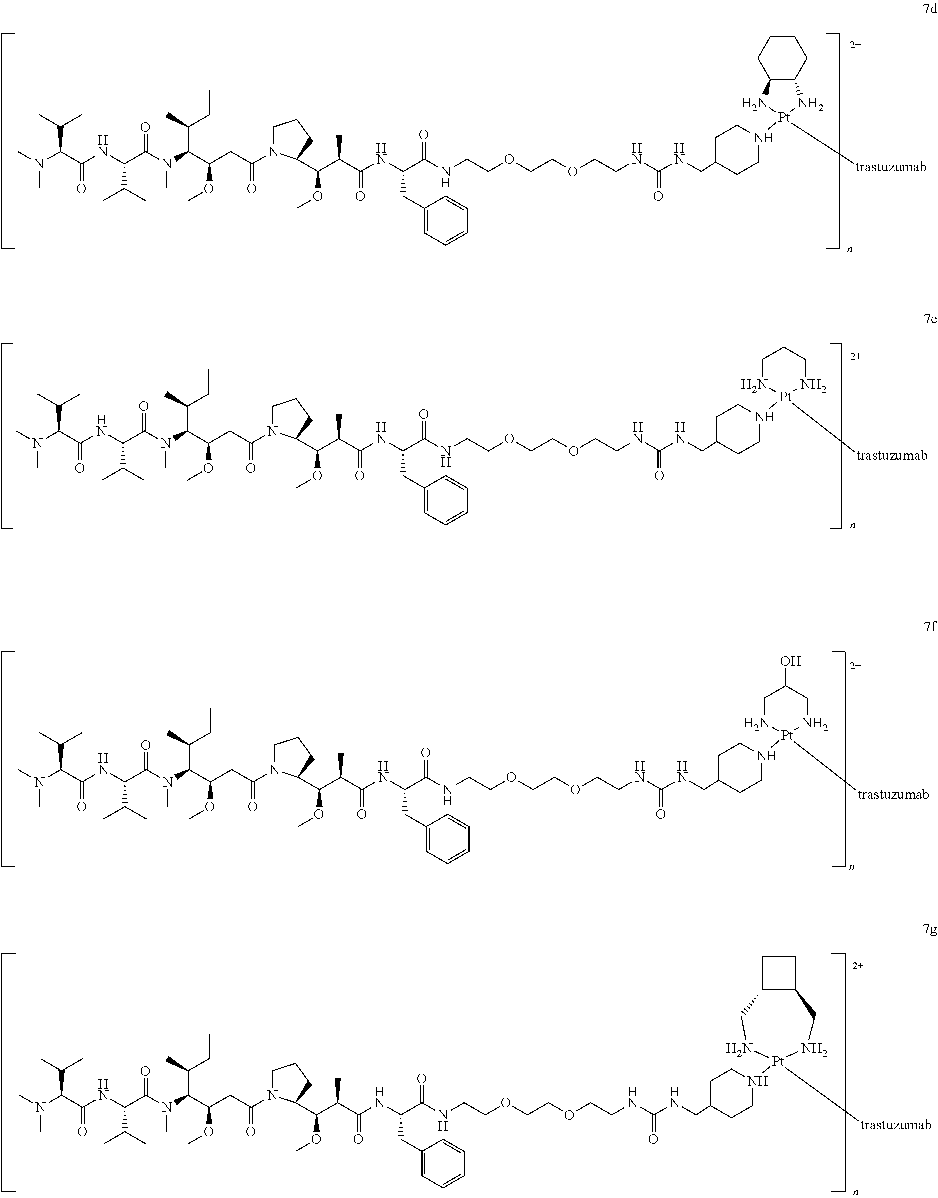

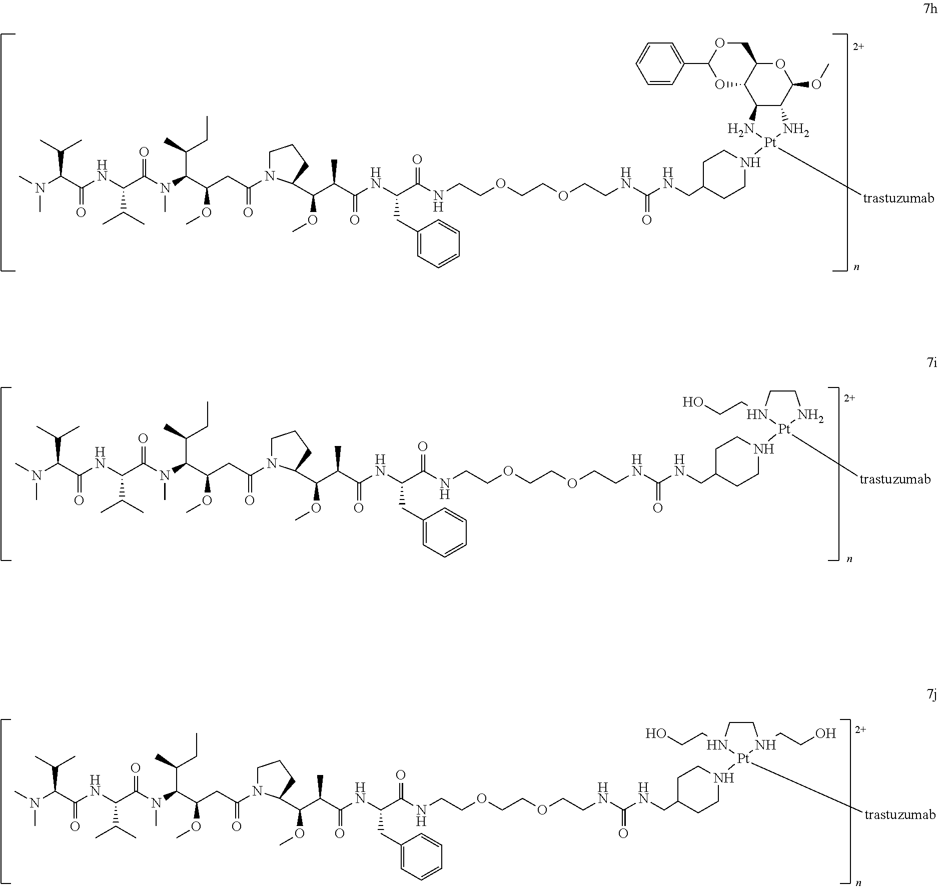

### 7.1. Synthesis and Analytical Characterization of the Bioconjugate Trastuzumab-[Pt((Fe)DFO-suc-pip)(ethane-1,2-diamine)]ₙ (7a); n=0-6

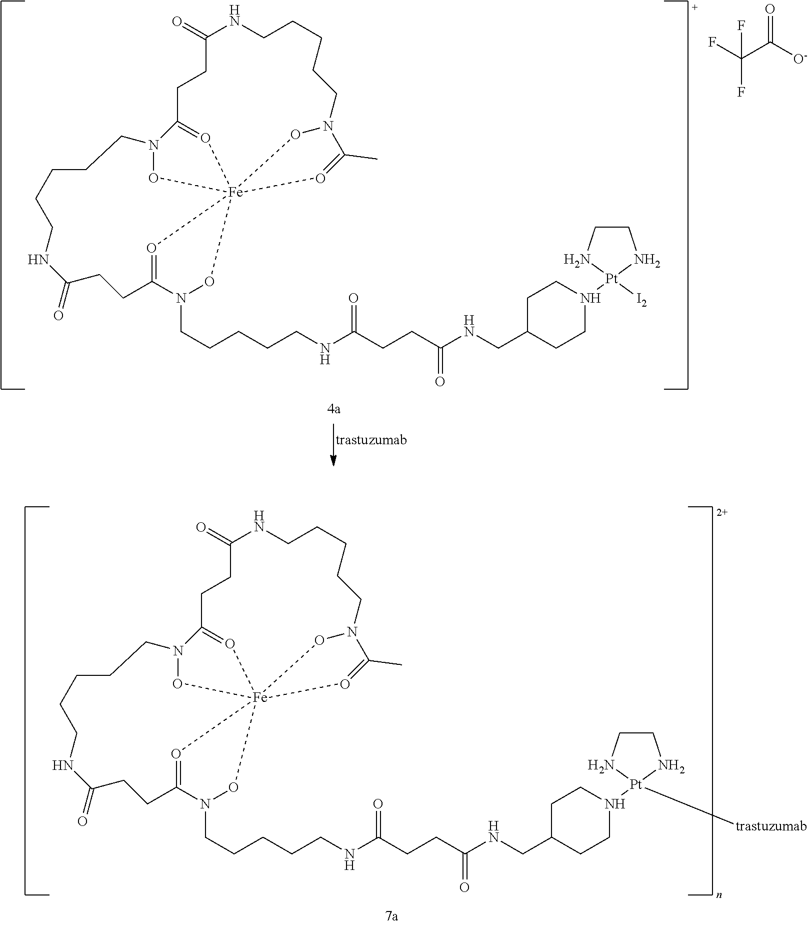

Trastuzumab (Herceptin®; 35.5 μL, 21 mg/mL, 1.0 eq.), rebuffered from the pharmacy storage buffer to PBS by spin filtration, was diluted with 200 mM HEPES buffer (6.15 μL, pH 8.1) containing 100 mM NaI, and [PtCl((Fe)DFO-suc-pip)(ethane-1,2-diamine)]⁺ TFA⁻ (4a) (20.0 μL, 825 μM in 20 mM NaCl, 3.3 eq.) was added. The sample was incubated in a thermoshaker at 47° C. for 24 h, followed by addition of a solution of thiourea (61.7 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC (after removal of Fe(III) using EDTA): 96.8% monomer. SEC-MS analysis was performed after purification of the conjugate 7a to determine the DAR:DAR=2.18 (corresponds to 66% conjugation efficiency). The complex distribution on the fragments of trastuzumab was determined by SDS-PAGE/phosphorimager analysis: % Hc=87%, % Lc=13%, % F(ab′)₂=30%.

### 7.2. Synthesis and Analytical Characterization of the Bioconjugate trastuzumab-[Pt(auristatin F-(4-(12-amino-3-oxo-7,10-dioxa-2,4-diazadodecyl)piperidine))(ethane-1,2-diamine)]ₙ (7b); n=0-6

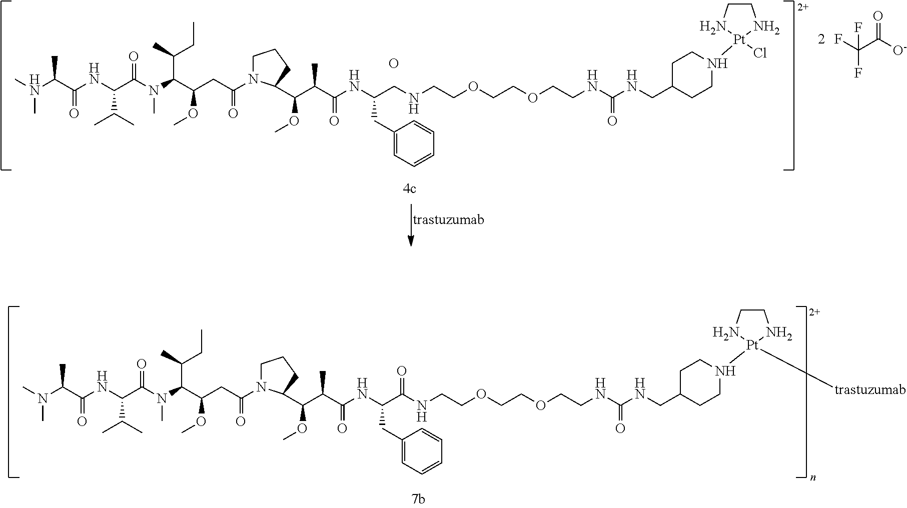

Trastuzumab (Herceptin®; 35.5 μL, 21 mg/mL, 1.0 eq.), rebuffered from the pharmacy storage buffer to PBS by spin filtration, was diluted with 200 mM HEPES buffer (6.15 μL, pH 8.1) containing 100 mM of NaI solution, and Pt(auristatin F-(4-(12-amino-3-oxo-7,10-dioxa-2,4-diazadodecyl)piperidine))Cl(ethane-1,2-diamine) (4c) (20.0 μL, 825 μM in 20 mM NaCl, 3.3 eq.) was added. The sample was incubated in a thermoshaker at 47° C. for 24 h, followed by the addition of a solution of thiourea (61.7 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.2% monomer. SEC-MS analysis was performed after purification of the conjugate 7b to determine the DAR and the complex distribution on the fragments of trastuzumab: DAR=2.81 (corresponds to 85% conjugation efficiency), % Hc=87%, % Lc=13%, % F(ab′)₂=22%, % Fab=15%, % Fc=85%.

### 7.3. Synthesis and Analytical Characterization of the Bioconjugate trastuzumab-[Pt(auristatin F-(4-(12-amino-3-oxo-7,10-dioxa-2,4-diazadodecyl)piperidine))((1R,2R)-(−)-1,2-diaminocyclohexane)]ₙ (7c)

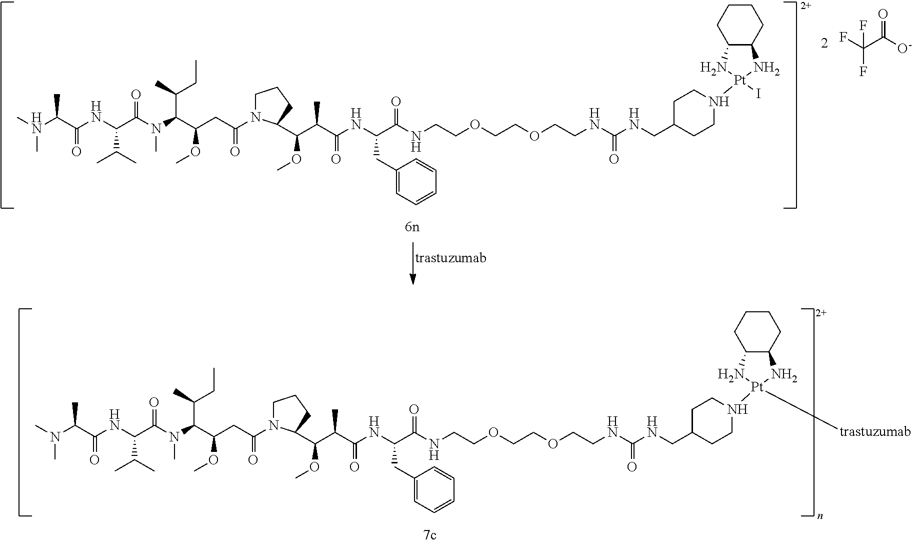

Trastuzutnab (Herceptin®; 71 μL, 21 mg/mL, 1.0 eq.), rebuffered from the pharmacy storage buffer to PBS by spin filtration, was diluted with MilliQ water (33.4 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I((1R,2R)-(−)-1,2-diaminocyclohexane) (6n) (6.6 μL, 5 mM in 20 mM NaI, 3.3 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.1% monomer. SEC-MS analysis was performed after purification of the conjugate 7c to determine the DAR:DAR=3.3 (corresponds to a quantitative conjugation efficiency).

### 7.4. Synthesis and Analytical Characterization of the Bioconjugate trastuzumab-[Pt(auristatin F-(4-(12-amino-3-oxo-7,10-dioxa-2,4-diazadodecyl)piperidine))((1S,2S)-(−)-1,2-diaminocyclohexane)], (7d)

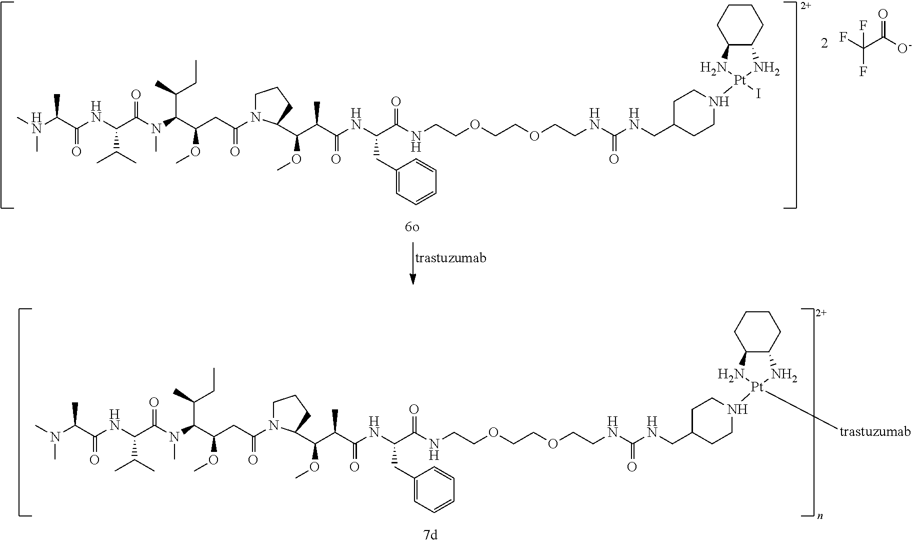

Trastuzumab (Herceptin®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I(((1S,2S)-(−)-1,2-diaminocyclohexane)) (6o) (6 μL, 5 mM in 20 mM NaI, 3.0 eq.) was added. The sample was incubated in a thermoshaker at 47° C. for 24 h, followed by the addition of a solution of thiourea. (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.5% monomer. SEC-MS analysis was performed after purification of the conjugate 7d to determine the DAR:DAR=3.0 (corresponds to 91% conjugation efficiency).

### 7.5. Synthesis and Analytical Characterization of the Bioconjugate trastuzumab-[Pt(auristatin F-(4-(12-amino-3-oxo-7,10-dioxa-2,4-diazadodecyl)piperidine))(propane-1,3-diamine)]ₙ (7e)

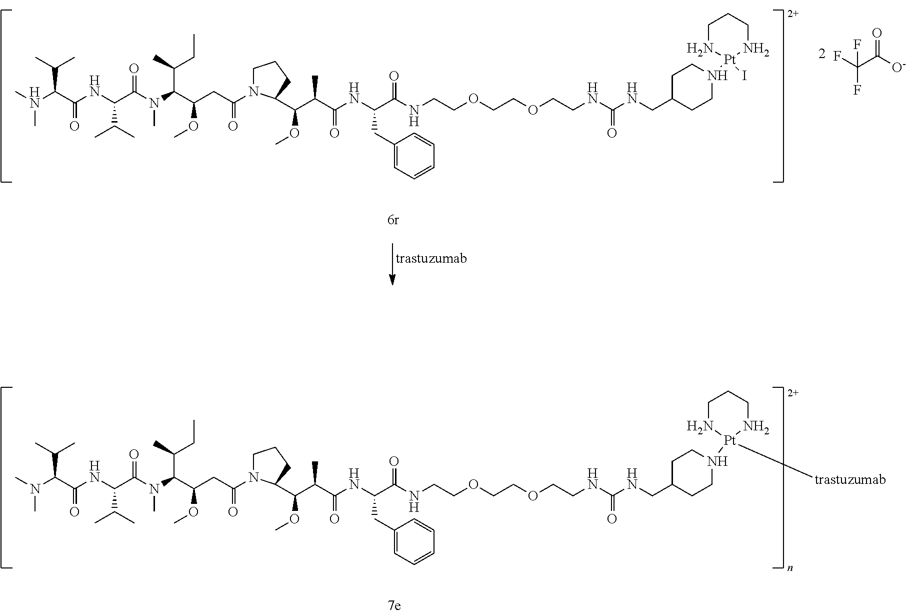

Trastuzumab (Herceptin®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I(propane-1,3-diamine) (6r) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 96.3% monomer. SEC-MS analysis was performed after purification of the conjugate 7e to determine the DAR:DAR=2.7 (corresponds to 90% conjugation efficiency).

### 7.6. Synthesis and Analytical Characterization of the Bioconjugate trastuzumab-[Pt(auristatin F-(4-(12-amino-3-oxo-7,10-dioxa-2,4-diazadodecyl)piperidine))(1,3-diaminopropan-2-ol)]ₙ (7f)

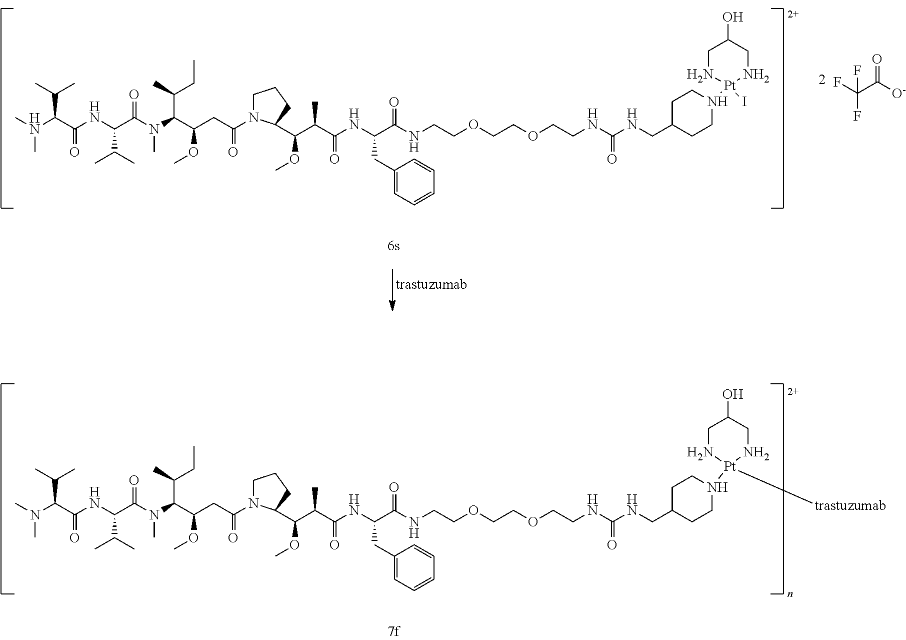

Trastuzumab (Herceptin®; 238 μL, 21 mg/mL, 1.0 eq.), rebuffered from the pharmacy storage buffer to PBS by spin filtration, was diluted with MilliQ water (105.6 μL) and 200 mM HEPES buffer (41.2 μL, pH 8.1) containing 100 mM of NaI solution, and Pt(auristatin F-(4-(12-amino-3-oxo-7,10-dioxa-2,4-diazadodecyl)piperidine))I(1,3-diaminopropan-2-ol) (6s) (28.5 μL, 5 mM in 20 mM NaI, 4.2 eq.) was added. The sample was incubated in a thermoshaker at 47° C. for 2 h, followed by the addition of a solution of thiourea (411 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.9% monomer. SEC-MS analysis was performed after purification of the conjugate 7f to determine the DAR:DAR=2.7 (corresponds to 64% conjugation efficiency).

### 7.7. Synthesis and Analytical Characterization of the Bioconjugate trastuzumab-[Pt(auristatin F-(4-(12-amino-3-oxo-7,10-dioxa-2,4-diazadodecyl)piperidine))((1R,2R)-cyclobutane-1,2-diyl)dimethanamine)]ₙ (7g)

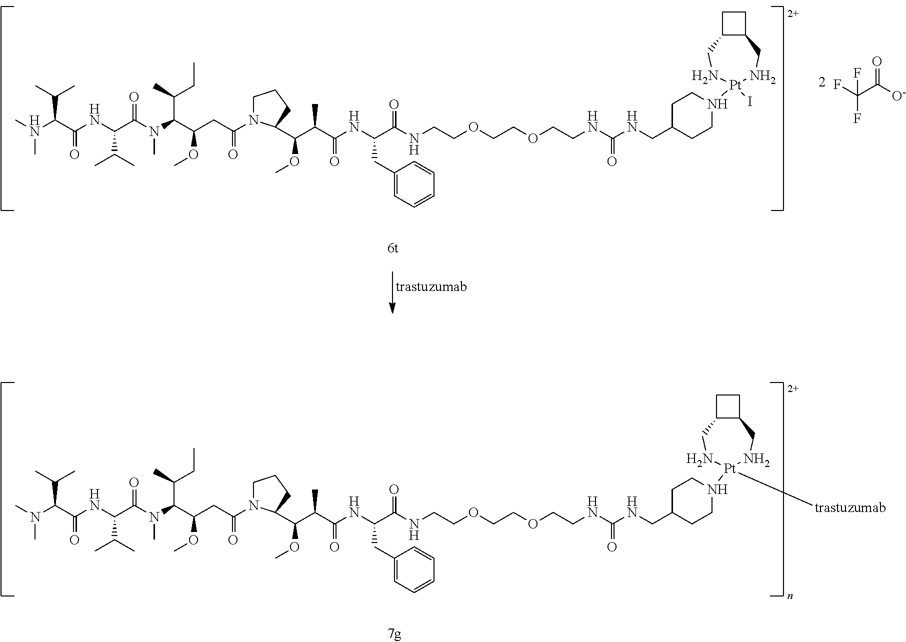

Trastuzumab (Herceptin®; 71 μL, 21 mg/mL, 1.0 eq.), rebuffered from the pharmacy storage buffer to PBS by spin filtration, was diluted with MilliQ water (33.4 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I((1R,2R)-cyclobutane-1,2-diyl)dimethanamine) (6t) (6.6 μL, 5 mM in 20 mM NaI, 3.3 eq.) was added. The sample was incubated in a thermoshaker at 47° C. for 24 h, followed by the addition of a solution of thiourea. (123.3 μL, 20 mM in H₂O) and incubation at 37° C. for 30 min. The conjugate was purified by PD-10 column (equilibrated with phosphate buffered saline), followed by spin filtration using kD MWCO filters (washed 4× with PBS buffer), after which it was reconstituted and stored in PBS buffer.

The antibody integrity was controlled by SEC: 98.0% monomer. SEC-MS analysis was performed after purification of the conjugate 7g to determine the DAR:DAR=3.0 (corresponds to 91% conjugation efficiency).

### 7.8. Synthesis and Analytical Characterization of the Bioconjugate trastuzumab-[Pt(auristatin F (4-(12-amino-3-oxo-7,10-dioxa-2,4-diazadodecyl)piperidine))((4aR,6R,7R,8R,8aS)-6-methoxy-2-phenylhexahydropyrano[3,2-d][1,3]dioxine-7,8-diamine]ₙ (7h)

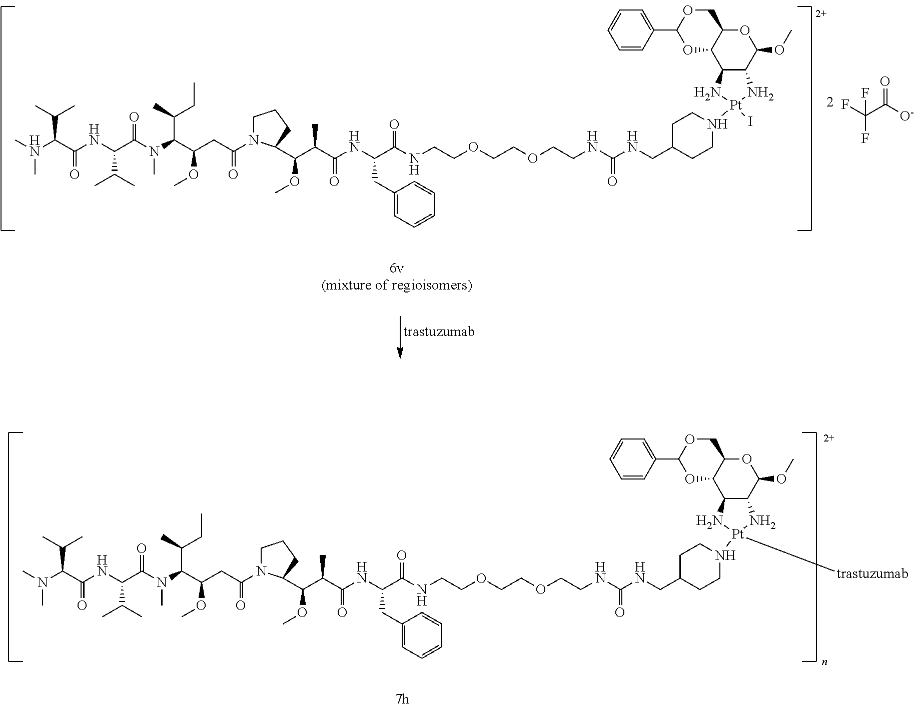

Trastuzutnab (Herceptin®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I((4aR,6R,7R,8R,8aS)-6-methoxy-2-phenylhexahydropyrano[3,2-d][1,3]dioxine-7,8-diamine) (6v) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 96.7% monomer. SEC-MS analysis was performed after purification of the conjugate 71i to determine the DAR:DAR=2.2 (corresponds to 73% conjugation efficiency).

### 7.9. Synthesis and Analytical Characterization of the Bioconjugate trastuzumab-[Pt(auristatin F (4-(12-amino-3-oxo-7,10-dioxa-2,4-diazadodecyl)piperidine))(2-((2-aminoethyl)amino)ethan-1-ol]ₙ (71)

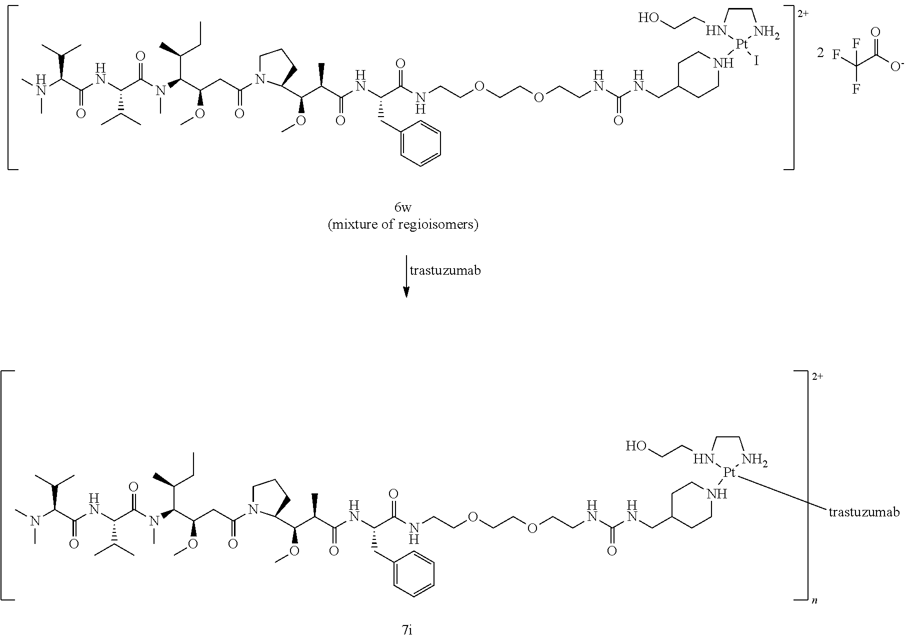

Trastuzumab (Herceptin®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I(2-((2-aminoethyl)amino)ethan-1-ol) (6w) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.4% monomer. SEC-MS analysis was performed after purification of the conjugate 7i to determine the DAR:DAR=2.8 (corresponds to 93% conjugation efficiency).

### 7.10. Synthesis and Analytical Characterization of the Bioconjugate trastuzumab-[Pt(auristatin F-(4-(12-amino-3-oxo-7,10-dioxa-2,4-diazadodecyl)piperidine))(2,2′-(ethane-1,2-diylbis(azanediyl))bis(ethan-1-ol)]ₙ (7j)

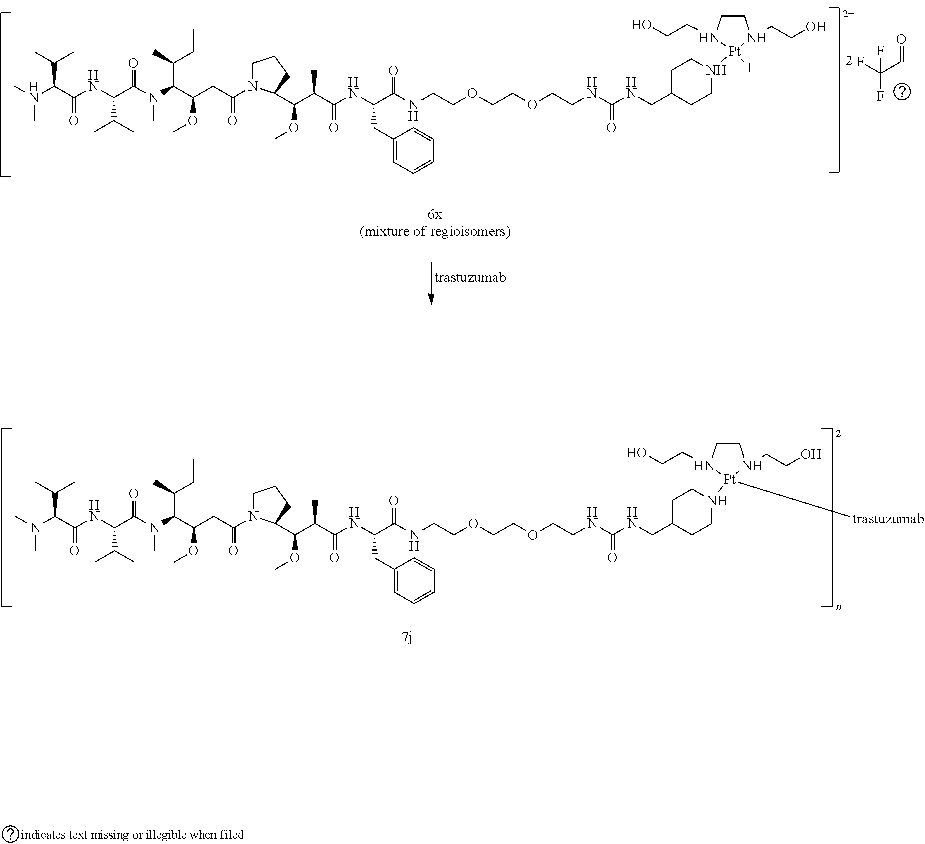

Trastuzumab (Herceptin®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 μL, pH 8.1) containing 100 mM of NaI solution, and Pt(auristatin F-(4-(12-amino-3-oxo-7,10-dioxa-2,4-diazadodecyl)piperidine))I(2,2′-(ethane-1,2-diylbis(azanediyl))bis(ethan-1-ol)) (6x) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.3% monomer. SEC-MS analysis was performed after purification of the conjugate 7j to determine the DAR:DAR=2.6 (corresponds to 87% conjugation efficiency).

### Example 8: Examples of Azide-Bearing Trastuzumab-Lx Conjugates 8a-b Obtained from the “Semi-Final” Compounds (SFMs) 6z and 6aa for Use in the Copper-Free Click Chemistry

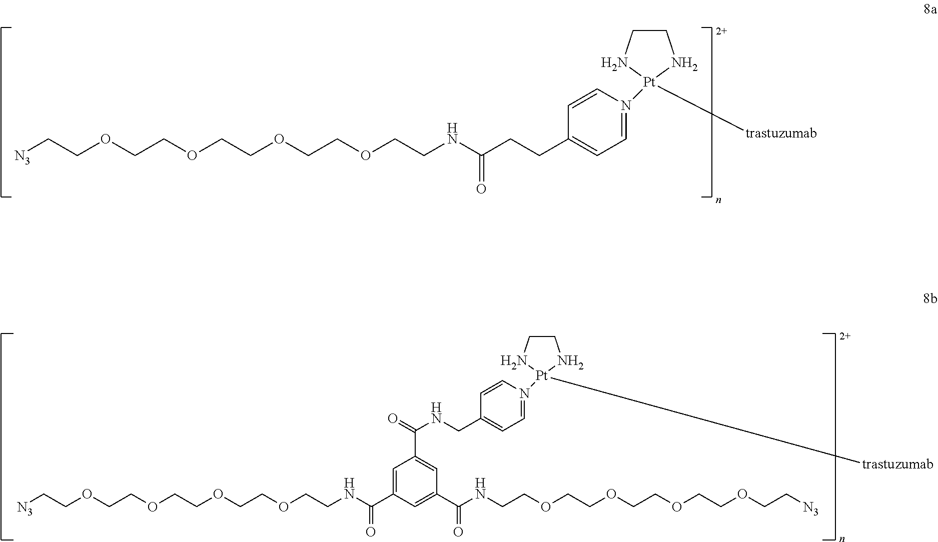

### 8.1. Synthesis and Analytical Characterization of the Bioconjugate trastuzumab-[Pt(N³-PEG₄-py)(ethane-1,2-diamine)]ₙ (8a)

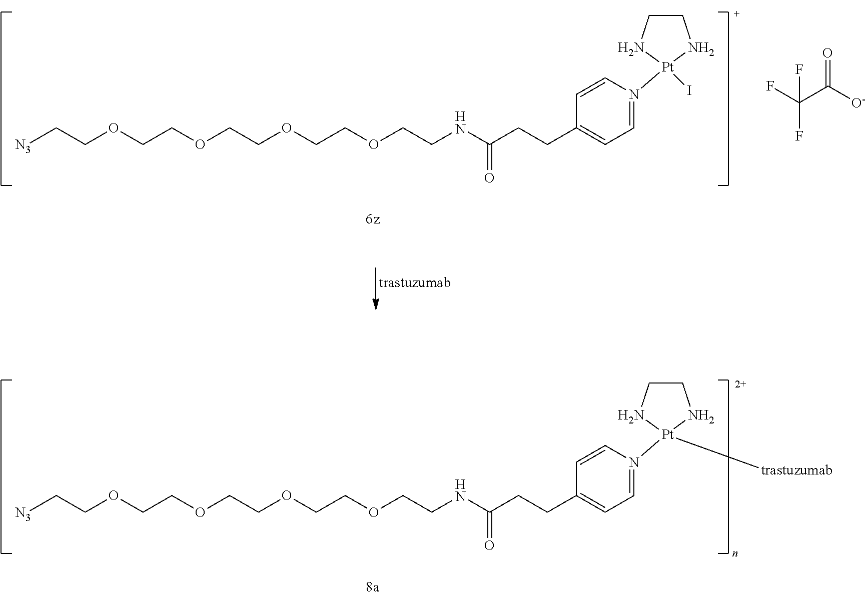

Trastuzumab (Herceptin®; 238 μL, 21 mg/mL, 5.0 mg, 33 nmol, 1.0 eq.), rebuffered from the pharmacy storage buffer to PBS by spin filtration, was diluted with 200 mM HEPES buffer (41.2 μL, pH 8.1) containing 100 mM of NaI solution, and [N-(14-azido-3,6,9,12-tetraoxatetradecyl)-3-(pyridin-4-yl)propanamide-Pt(ethane-1,2-diamine)I]⁺ TFA⁻ (6z) (21.8 μL, 5 mM in 10 mM NaI, 109 nmol, 3.3 eq.) was added. The sample was further diluted with milliQ water (112.2 μL) and incubated in a thermoshaker at 47° C. for 24 h, followed by the addition of a solution of thiourea (413 μL, 20 mM in H₂O) and incubation at 37° C. for 30 min. The conjugate was purified by PD-10 column (equilibrated with phosphate buffered saline), followed by spin filtration using 30 kD MWCO filters (washed 1× with PBS buffer), after which it was reconstituted and stored in PBS buffer.

### 8.2. Synthesis and Analytical Characterization of the Bioconjugate trastuzumab-[Pt(N¹,N³-bis(14-azido-3,6,9,12-tetraoxatetradecyl)-N⁵-(pyridin-4-ylmethyl)benzene-1,3,5-tricarboxamide)(ethane-1,2-diamine)]ₙ (8b)

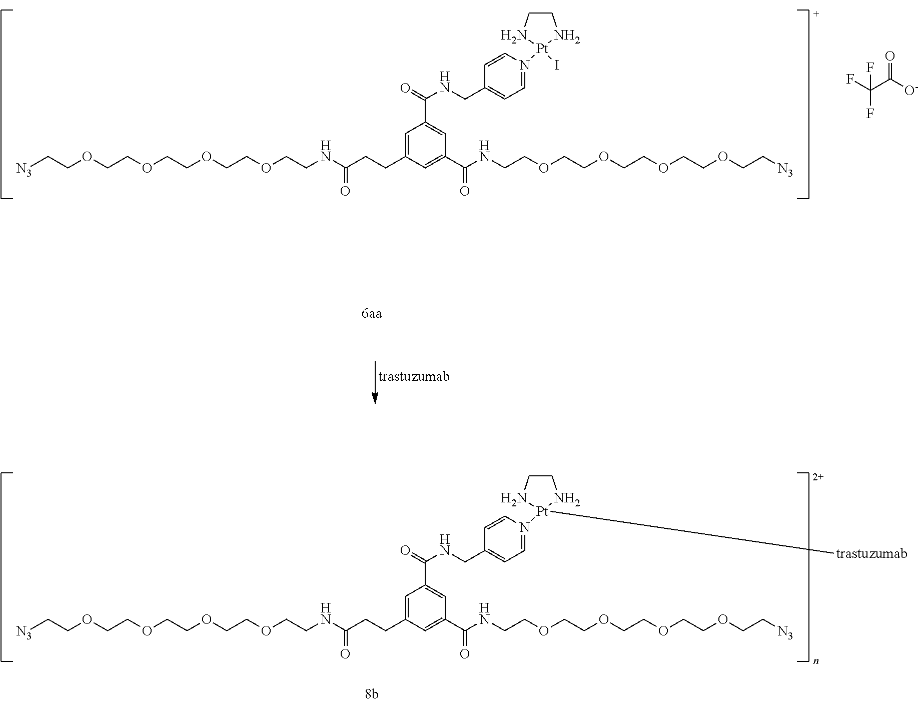

Trastuzumab (Herceptin®; 238 μL, 21 mg/mL, 5.0 mg, 33 nmol, 1.0 eq.), rebuffered from the pharmacy storage buffer to PBS by spin filtration, was diluted with 200 mM HEPES buffer (41.2 μL, pH 8.1) containing 100 mM of NaI solution, and [N¹,N³-bis(14-azido-3,6,9,12-tetraoxatetradecyl-Pt(ethane-1,2-diamine)I]⁺ TFA⁻ (6aa) (21.8 μL, 5 mM in 10 mM NaI, 109 nmol, 3.3 eq.) was added. The sample was further diluted with milliQ water (112.2 μL) and incubated in a thermoshaker at 47° C. for 24 h, followed by the addition of a solution of thiourea (413 μL, 20 mM in H₂O) and incubation at 37° C. for 30 min. The conjugate was purified by PD-10 column (equilibrated with phosphate buffered saline), followed by spin filtration using kD MWCO filters (washed 1× with PBS buffer), after which it was reconstituted and stored in PBS buffer.

### Example 9: Examples of Trastuzumab-Lx Conjugates 9a-f Obtained from the Conjugate 8a Via the Copper-Free Click Chemistry

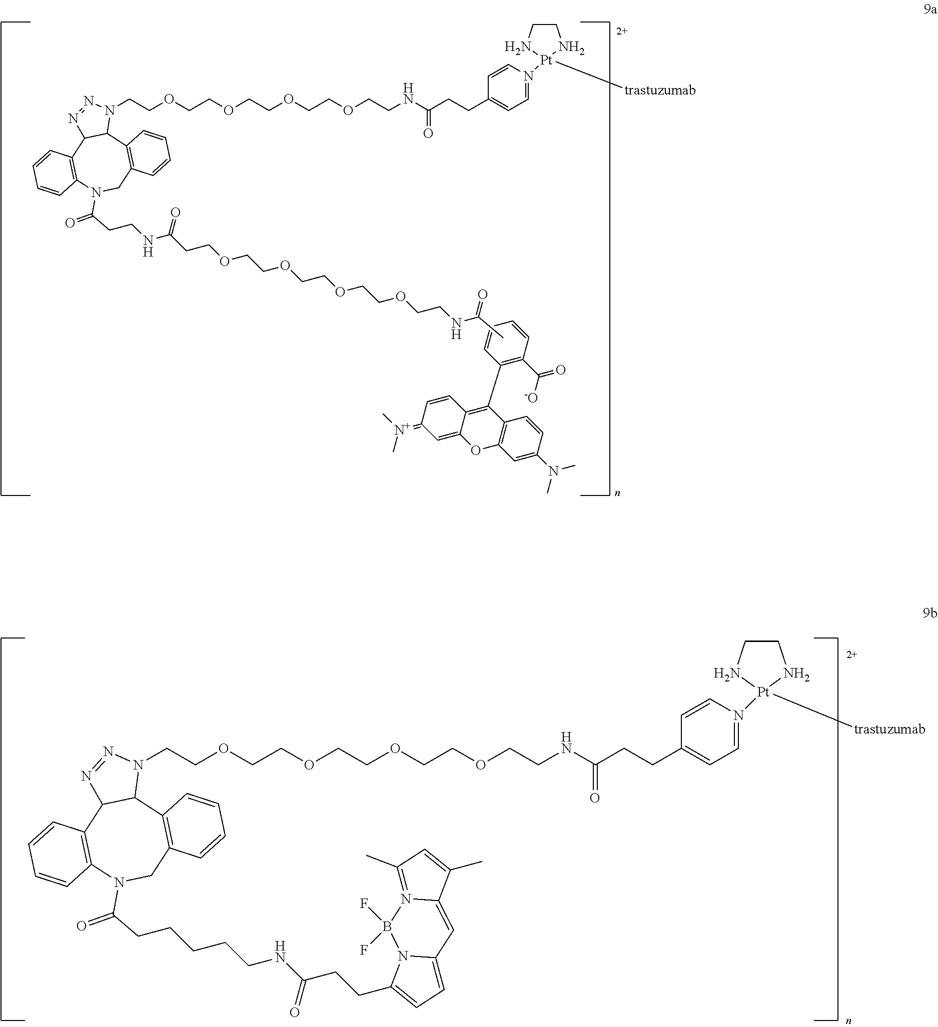

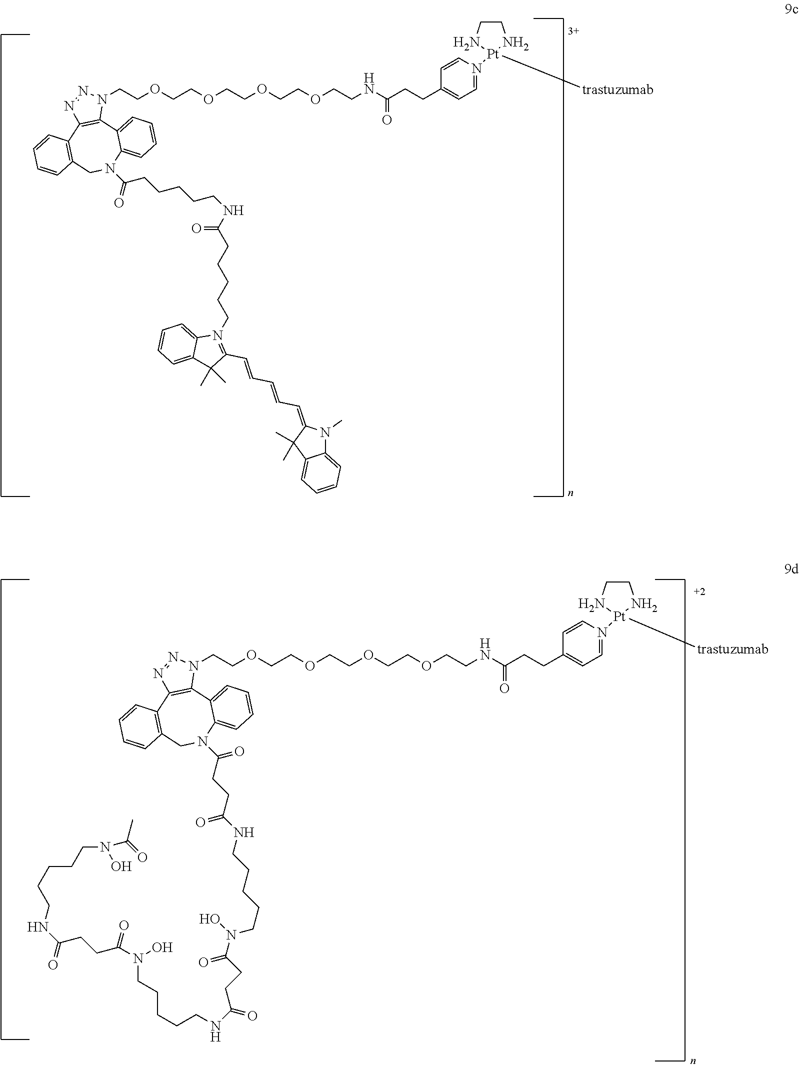

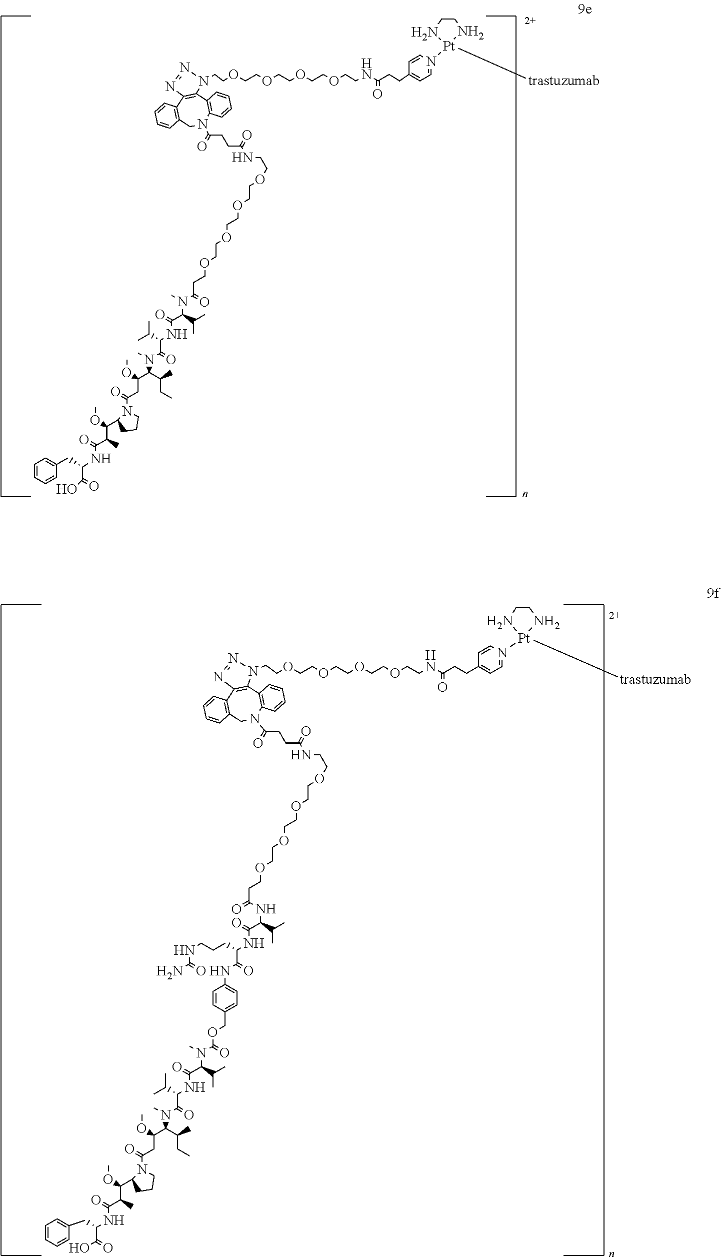

### 9.1. Synthesis of the Bioconjugate trastuzumab-[Pt(Fluor 545-PEG₄-DBCO-triazole-PEG₄-pyridine)]ₙ (9a)

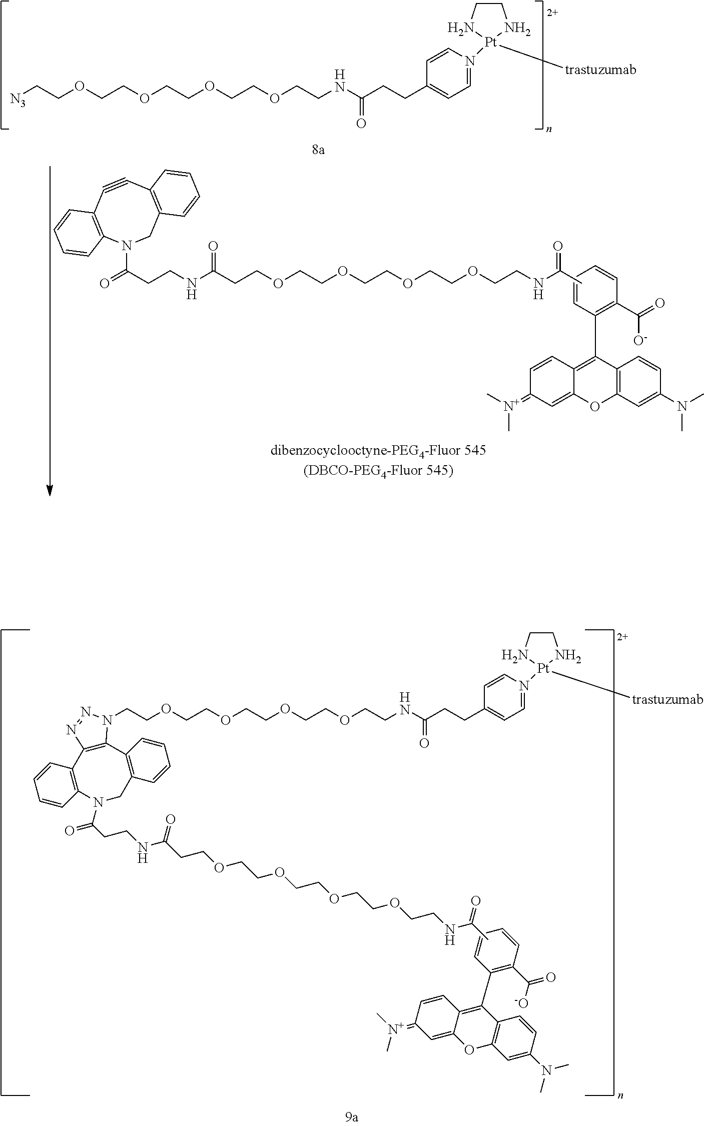

The bioconjugate 8a (303 μL, 4.95 mg/mL, 1.5 mg, 10 nmol, 1.0 eq.) was diluted with PBS (297 μL) and dibenzocyclooctyne-PEG₄-Fluor 545 (DBCO-PEG₄-Fluor 545; 10 μL, 10 mM in DMSO, 200 nmol, 20.0 eq.) was added. The sample was incubated in a thermoshaker at 37° C. 1.0 for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate which was 98.4% monomeric.

### 9.2. Synthesis of the Bioconjugate trastuzumab-[Pt(BODIPY FL-DBCO-triazole-PEG₄-pyridine)]ₙ (9b)

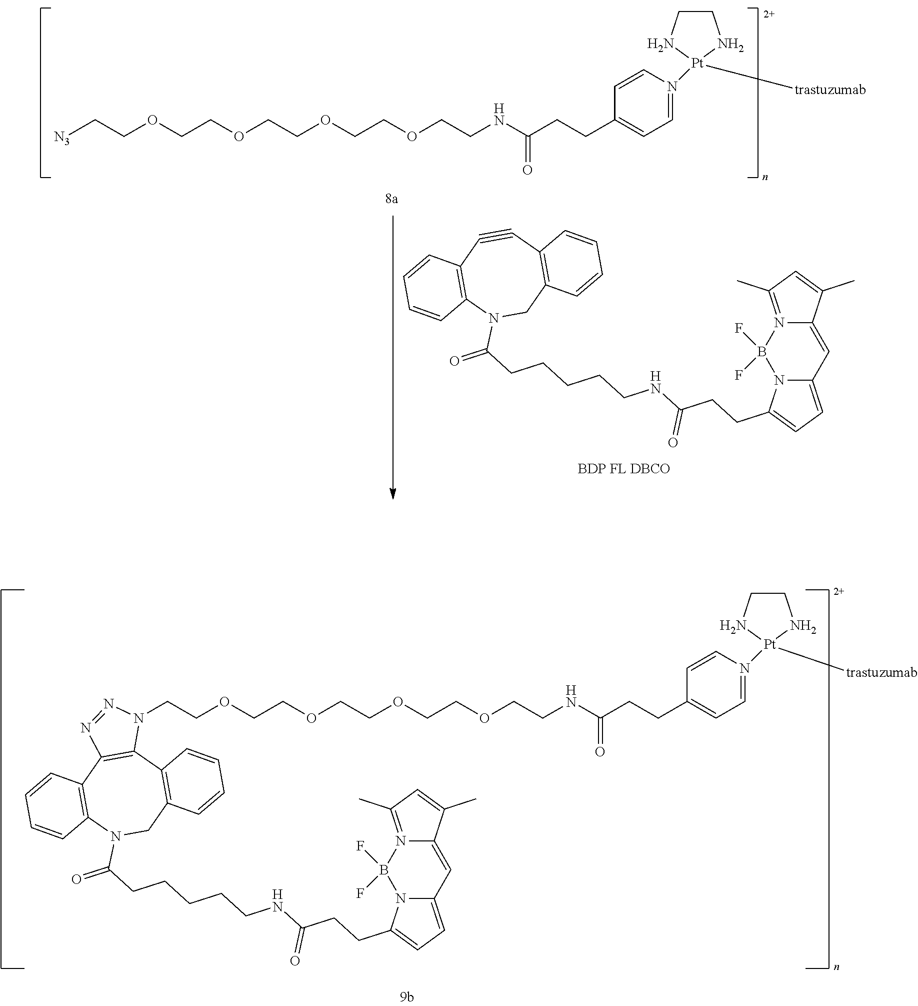

Bioconjugate 8a (57.6 μL, 4.34 mg/mL, 0.25 mg, 1.65 nmol, 1.0 eq.) was diluted with DMSO (57.6 μL) and BDP FL DBCO (2 μL, 10 mM in DMSO, 20 nmol, 12.1 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate which was 100% monomeric.

### 9.3. Synthesis of the Bioconjugate trastuzumab-[Pt(Cyanine5 DBCO-triazole-PEG₄-pyridine)]ₙ (9c)

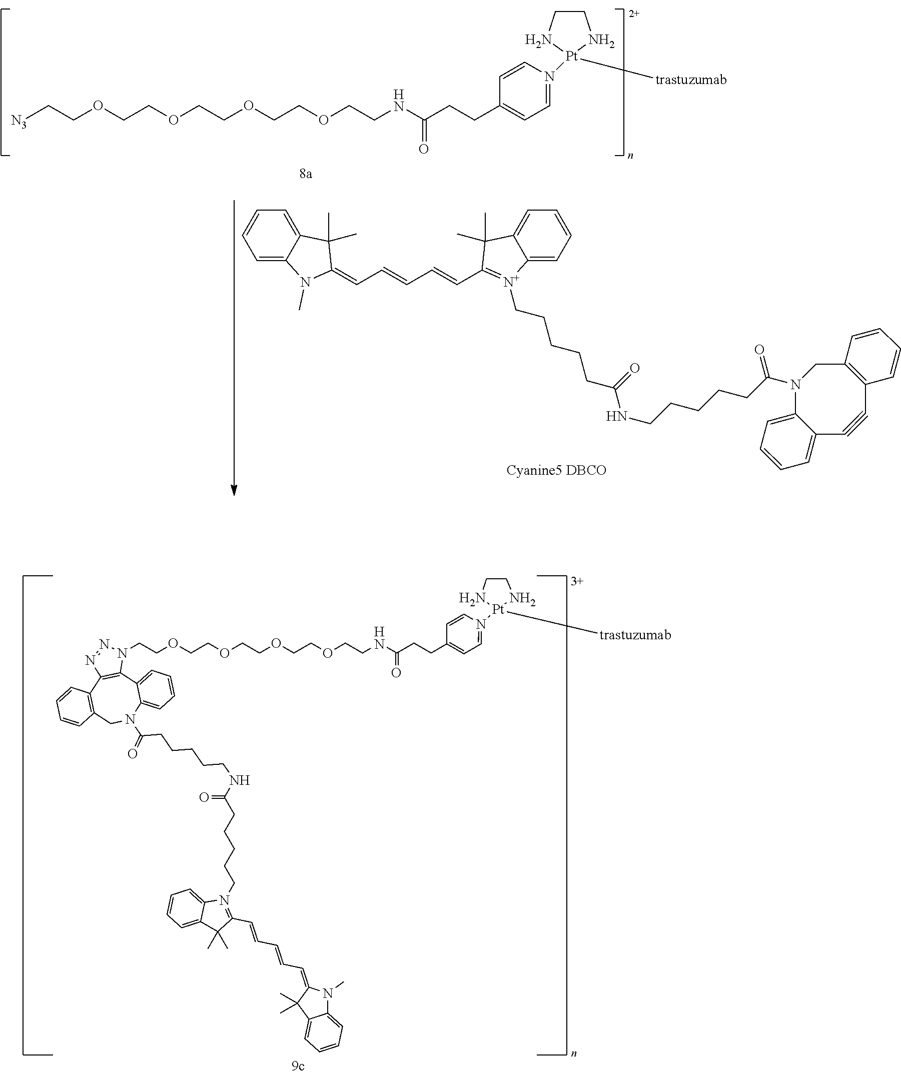

Bioconjugate 8a (57.6 μL, 4.34 mg/mL, 0.25 mg, 1.65 nmol, 1.0 eq.) was diluted with DMSO (57.6 μL) and Cyanine5 DBCO (2 μL, 10 mM in DMSO, 20 nmol, 12.1 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate which was 99.1% monomeric.

### 9.4. Synthesis of the Bioconjugate trastuzumab-[Pt(DFO-DBCO-triazole-PEG₄-pyridine)]ₙ (9d)

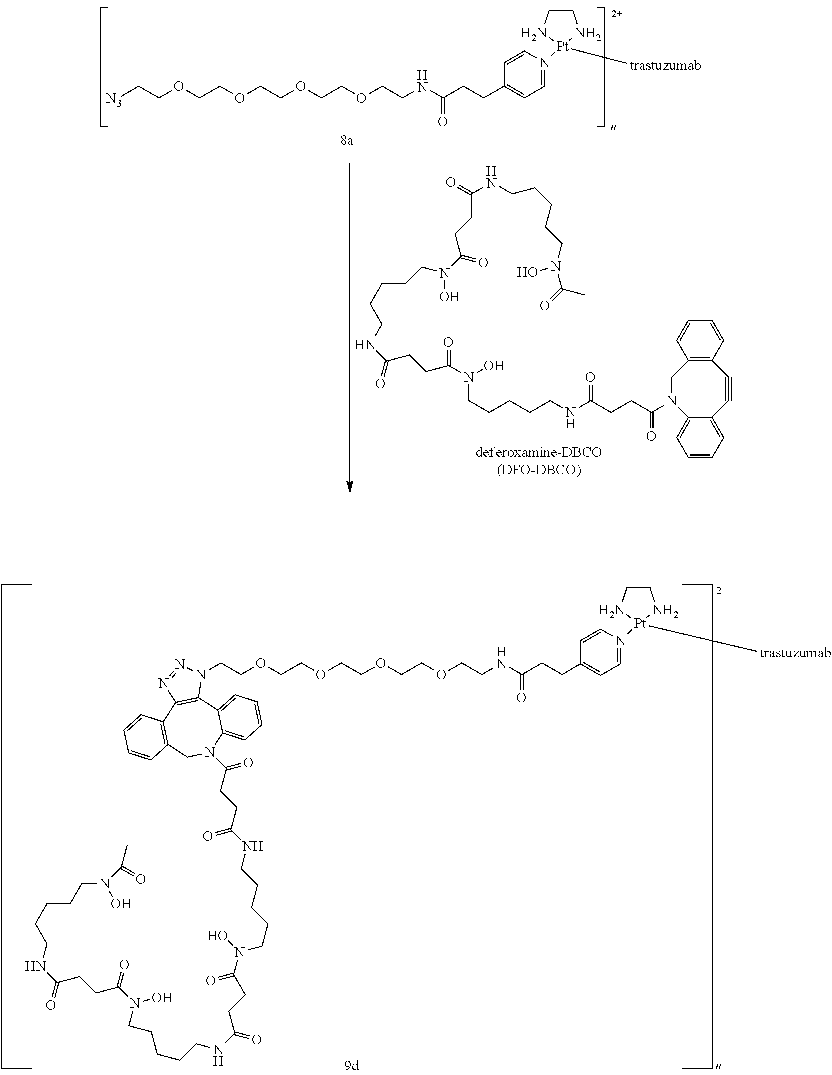

Bioconjugate 8a (300 μL, 5.0 mg/mL, 1.5 mg, 10 nmol, 1.0 eq.) was mixed with deferoxamine-DBCO (DFO-DBCO; 4 μL, 10 mM in DMSO, 40 nmol, 4.0 eq.). The sample was incubated in a thermoshaker at 25° C. for 2 h, after which the conjugate was purified by spin filtration using kD MWCO filters (washed 4× with 0.9% NaCl), after which it was reconstituted and stored in 0.9% NaCl buffer. The conjugation afforded a conjugate which was 97.8% monomeric.

### 9.5. Synthesis of the Bioconjugate trastuzumab-[Pt(MMAF-PEG₄-DBCO-triazole-PEG₄-pyridine)]ₙ (9e)

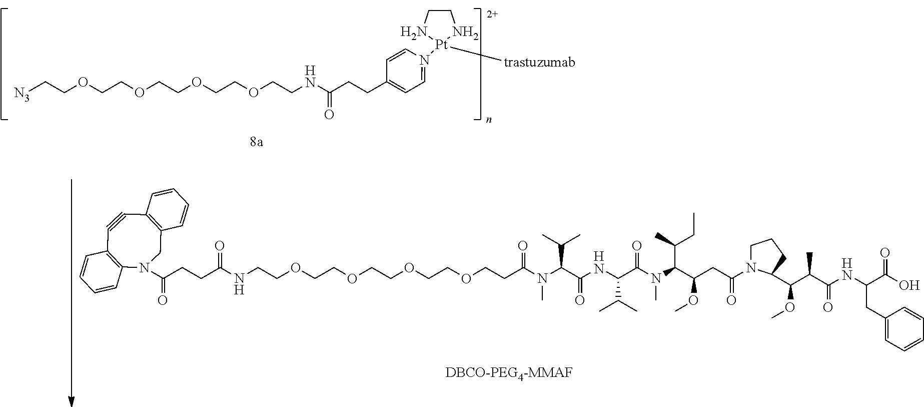

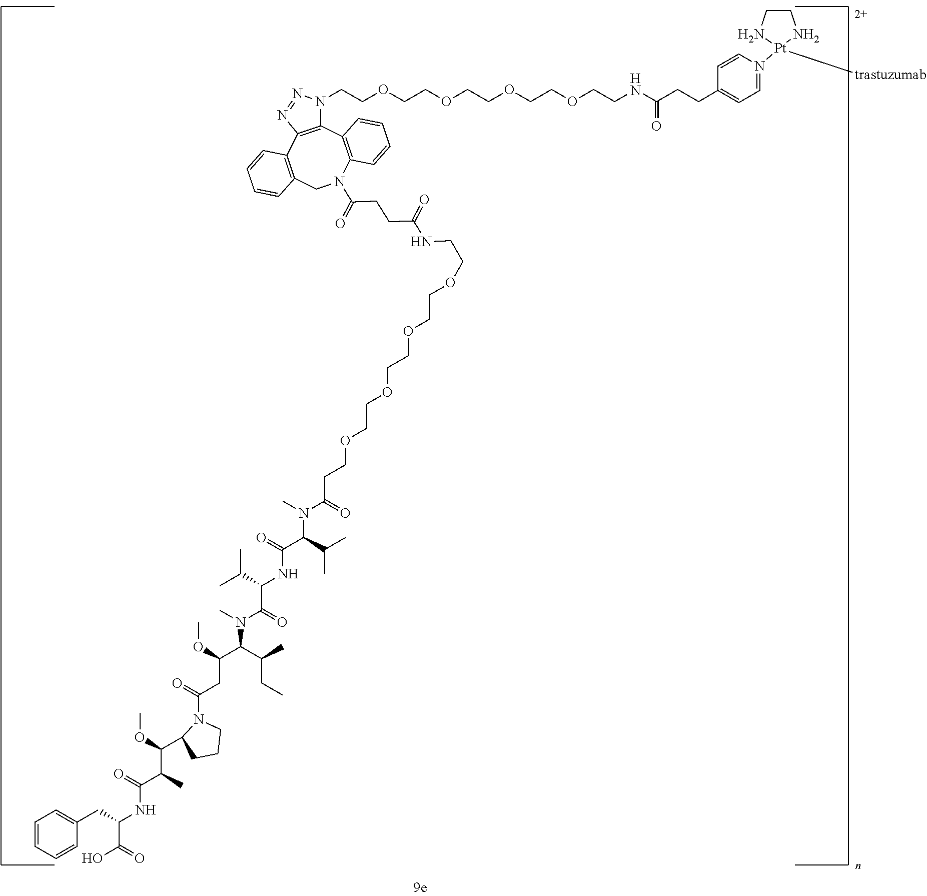

Bioconjugate 8a (300 μL, 5.0 mg/mL, 1.5 mg, 10 nmol, 1.0 eq.) was mixed with DBCO-PEG₄-MMAF (4 μL, 10 mM in DMSO, 40 nmol, 4.0 eq.). The sample was incubated in a thermoshaker at 25° C. for 2 h, after which the conjugate was purified by spin filtration using kD MWCO filters (washed 4× with PBS), after which it was reconstituted and stored in PBS buffer. The conjugation afforded a conjugate which was 97.4% monomeric and with a DAR of 2.4.

### 9.6. Synthesis of the Bioconjugate trastuzumab-[Pt(MMAF-PAB-vc-PEG₄-DBCO-triazole-PEG₄-pyridine)]ₙ (90

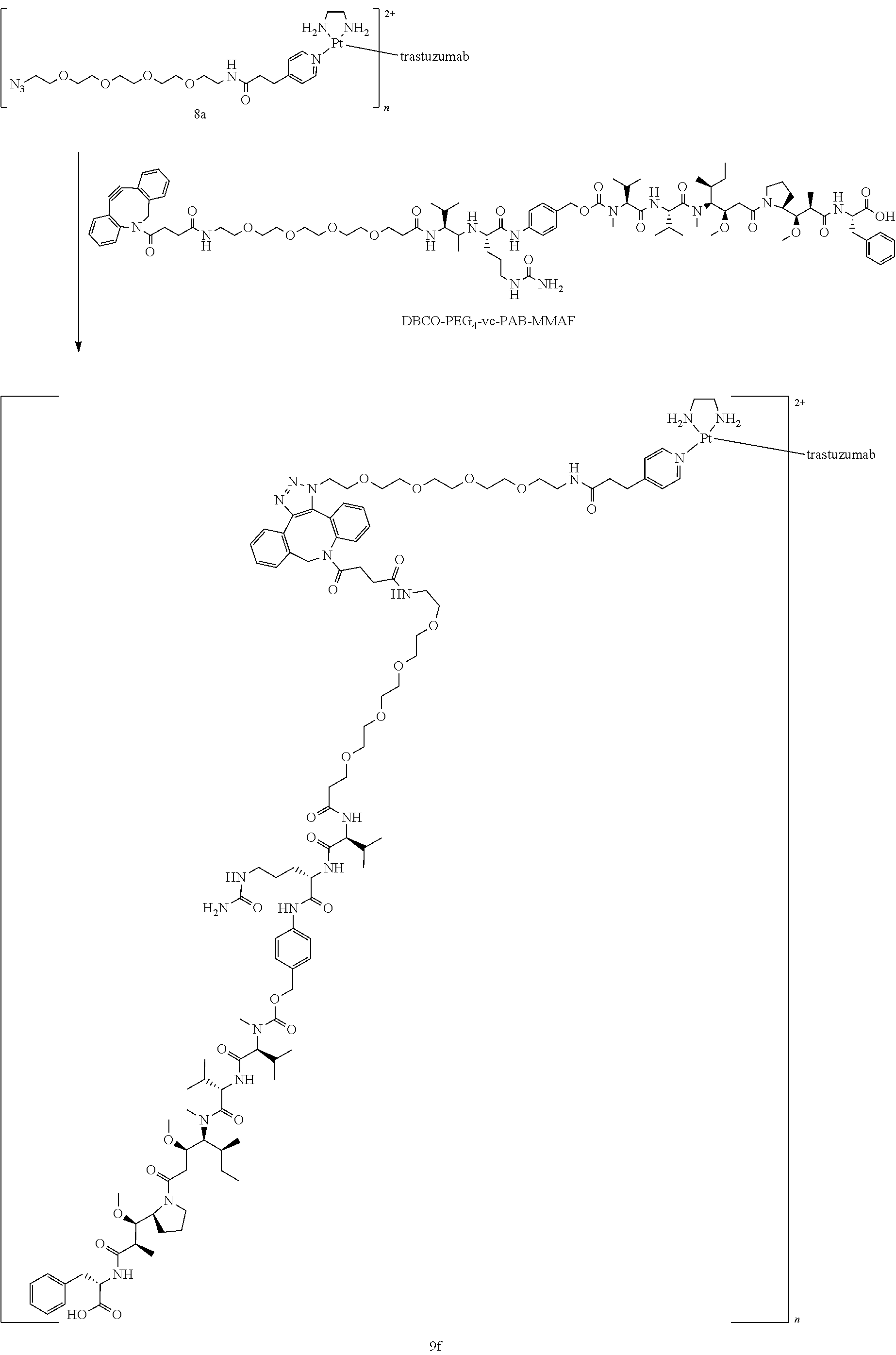

Bioconjugate 8a (300 μL, 5.0 mg/mL, 1.5 mg, 10 nmol, 1.0 eq.) was mixed with DBCO-PEG₄-vc-PAB-MMAF (4 μL, 10 mM in DMSO, 40 nmol, 4.0 eq.). The sample was incubated in a thermoshaker at 25° C. for 2 h, after which the conjugate was purified by spin filtration using kD MWCO filters (washed 4× with PBS), after which it was reconstituted and stored in PBS buffer. The conjugation afforded a conjugate which was 97.4% monomeric and with a DAR of 2.4.

### Example 10: Example of Trastuzumab-Lx Conjugate 10a Obtained from the Conjugate 8b Via the Copper-Free Click Chemistry

### 10.1. Synthesis of the Bioconjugate trastuzumab-[Pt((Fluor 545-PEG₄-DBCO-triazole-PEG₄)₂-benzene-pyridine)]ₙ (10a)

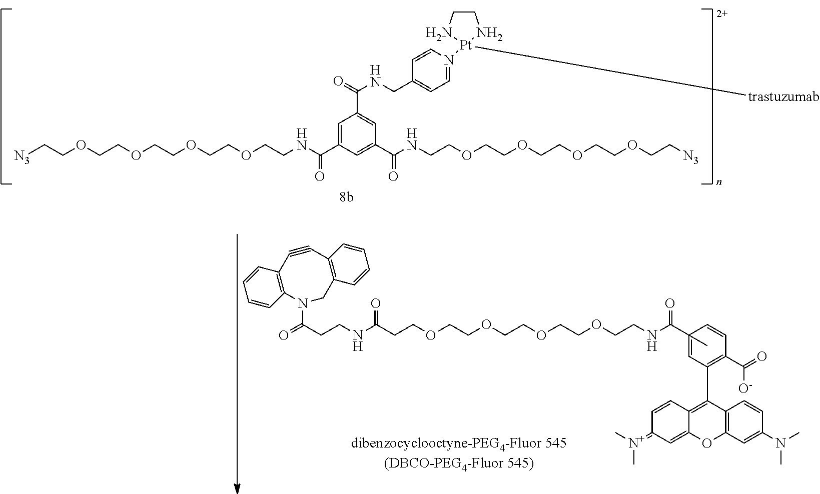

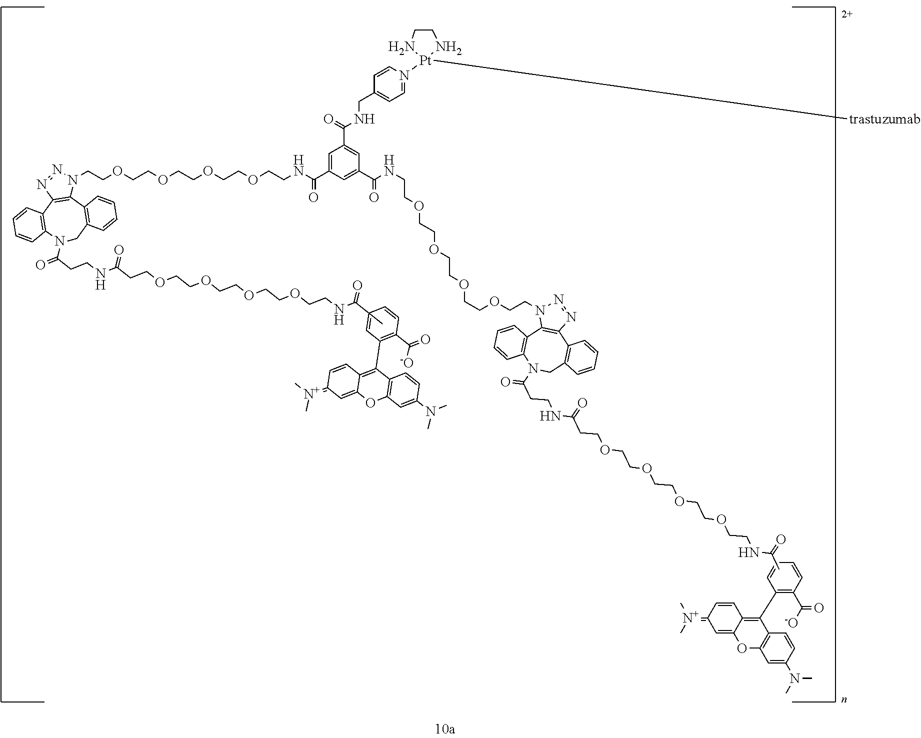

Bioconjugate 8b (303 μL, 4.95 mg/mL, 1.5 mg, 10 nmol, 1.0 eq.) was diluted with PBS (297 μL) and dibenzocyclooctyne-PEG₄-Fluor 545 (DBCO-PEG₄-Fluor 545; 20 μL, 10 mM in DMSO, 200 nmol, 20.0 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate which was 98.6% monomeric.

1. A secondary functional moiety according to formula I 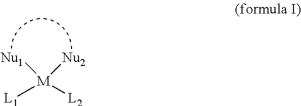
wherein
M is a transition metal complex,
one of the ligands L₁ or L₂ is iodide, bromide, or chloride and the other ligand is a primary functional moiety;
Nu is a nucleophilic group, wherein Nu₁ and Nu₂ can be the same groups or different groups and which together form a bidentate ligand, with the proviso that the bidentate ligand is not ethane-1,2-diamine.

2. The secondary functional moiety according to claim 1, wherein the bidentate ligand formed by Nu₁ and Nu₂ is represented by one of the following formulas:

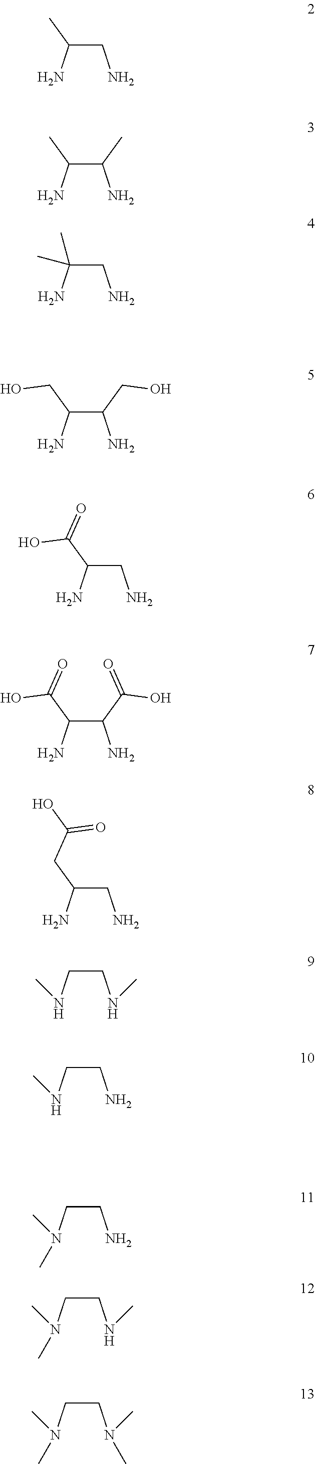

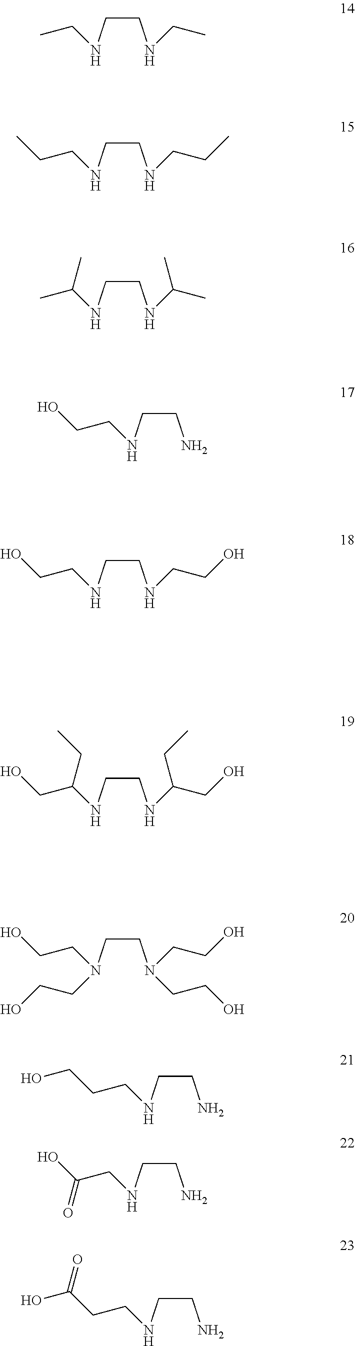

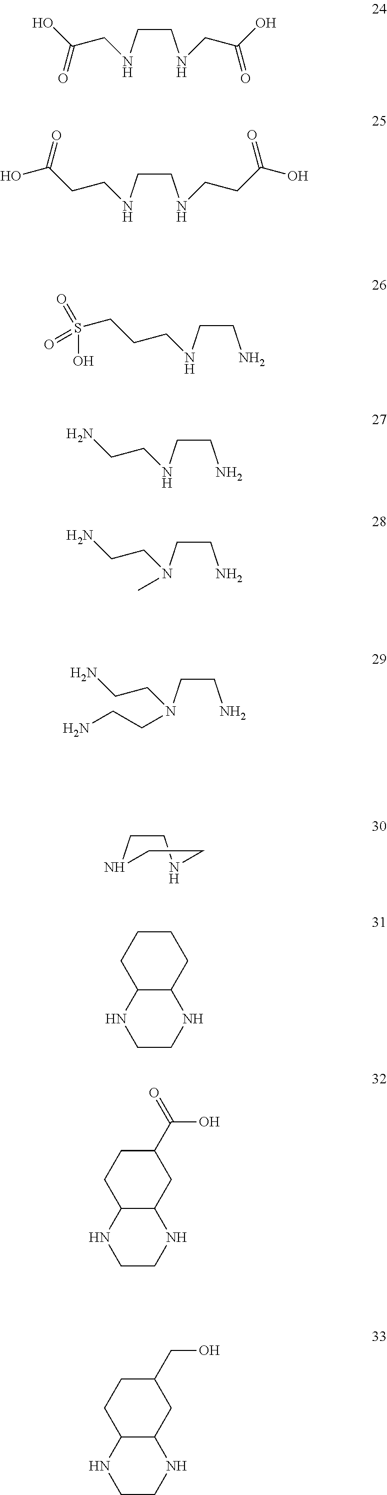

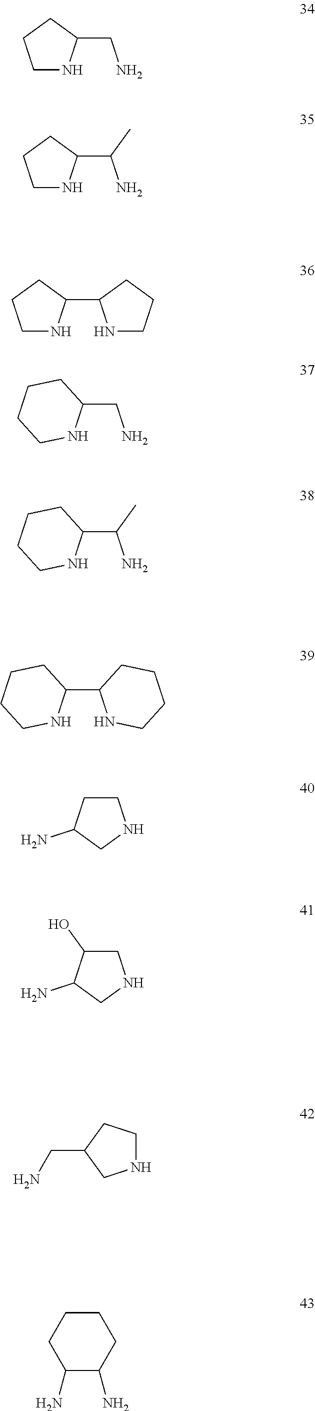

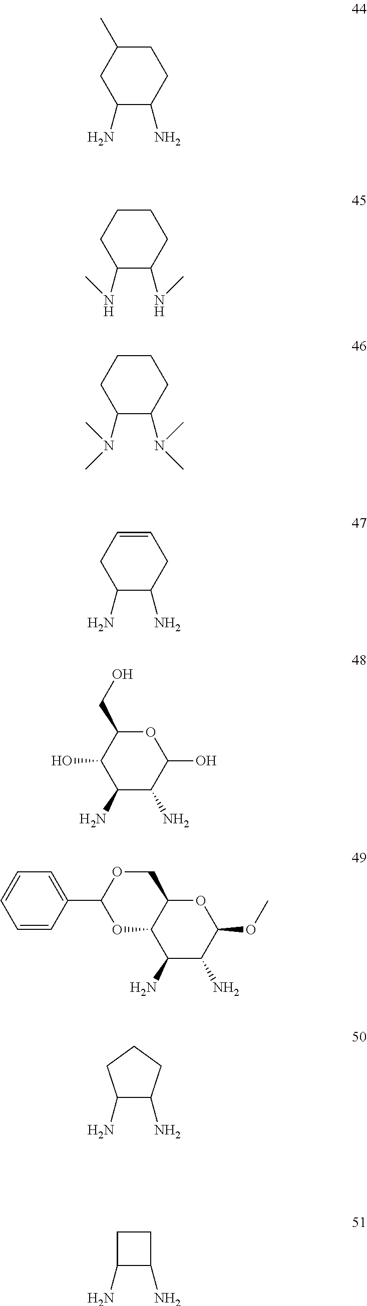

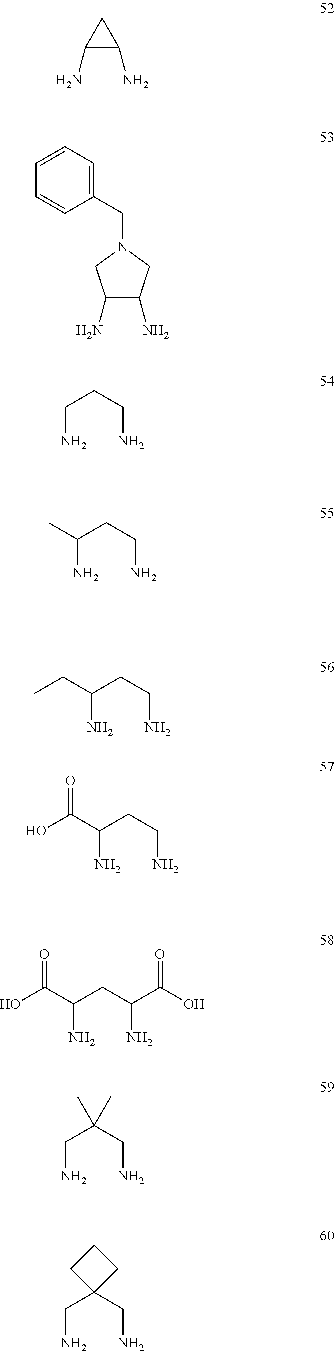

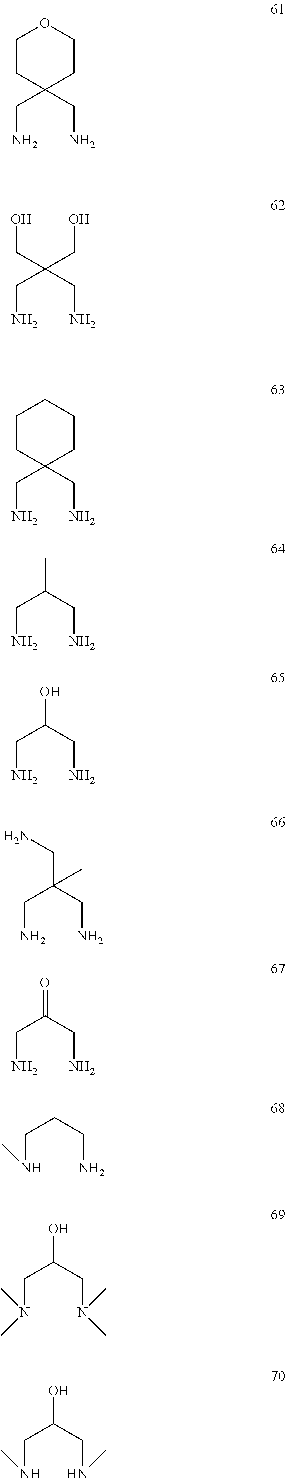

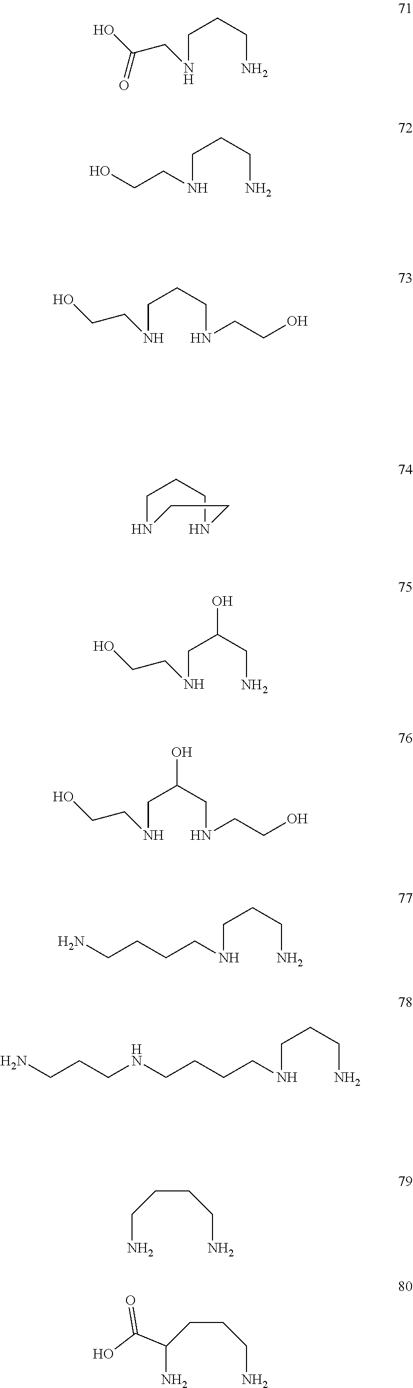

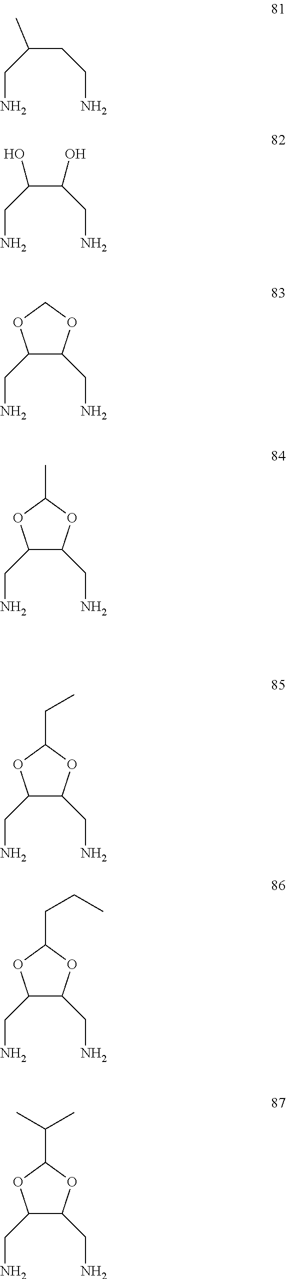

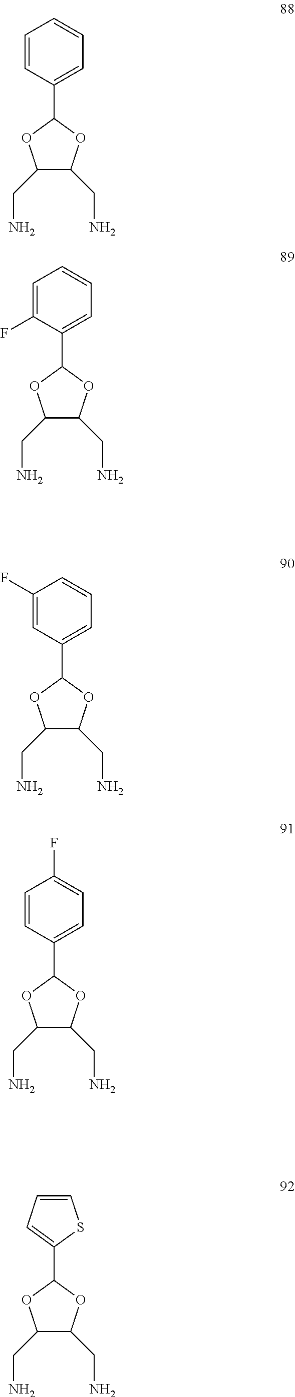 [SMILES: NCC1OC(c2ccccc2)OC1CN ; NCC1OC(c2ccccc2F)OC1CN ; NCC1OC(c2cccc(F)c2)OC1CN ; NCC1OC(c2ccc(F)cc2)OC1CN ; NCC1OC(c2cccs2)OC1CN]

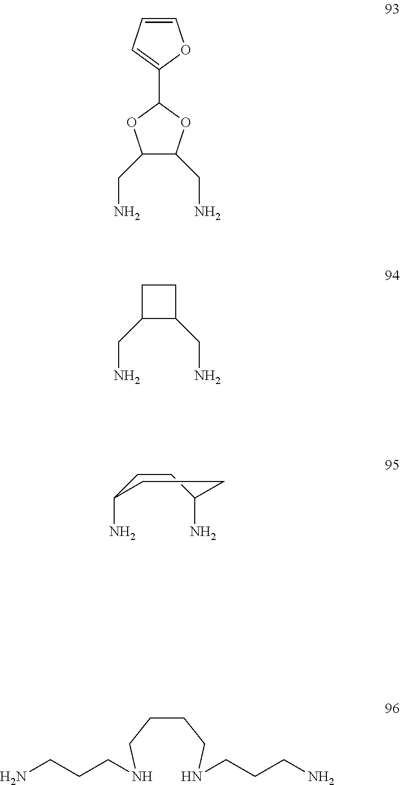 [SMILES: NCC1OC(c2ccco2)OC1CN ; NCC1CCC1CN ; NC1CCC(N)CC1 ; NCCCNCCCCNCCCN]

3. The secondary functional moiety of claim 1, wherein the bidentate ligand formed by Nu₁ and Nu₂ is represented by one of the following formulas:

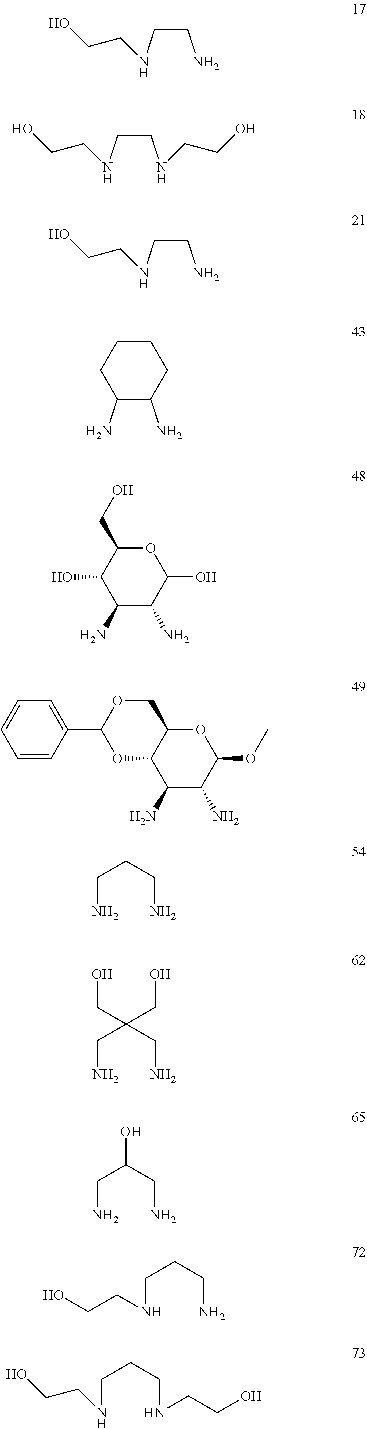

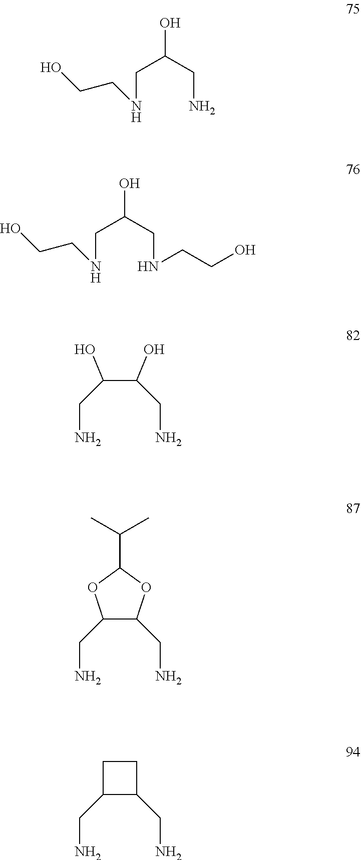

4. The secondary functional moiety of claim 1, wherein the bidentate ligand formed by Nu₁ and Nu₂ is represented by one of the following formulas:

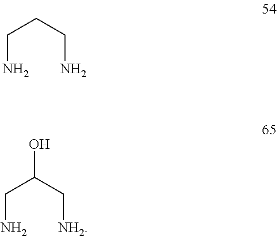 [SMILES: NCCCN ; NCC(O)CN]

5. The secondary functional moiety of claim 1, wherein the transition metal complex M is a platinum(II) complex.

6. The secondary functional moiety claim 1, wherein the primary functional moiety is selected from the group consisting of a therapeutic compound, a diagnostic compound, a chelating agent, a dye, a model compound, and a cytotoxic compound.

7. The secondary functional moiety of claim 6, wherein the primary functional moiety is a cytotoxic compound selected from the group consisting of auristatins, dolastatins, symplostatins, maytansinoids, tubulysins, HTI-286, calicheamycins, duocarmycins, pyrrolobenzodiazepines (PBDs), indolino-benzodiazepines (IGNs), camptothecin, anthracyclines, azonafides, amanitins, cryptophycins, rhizoxins, epothilones, spliceostatins, thailanstatins, colchicines, aplyronines, taxoids, methotrexate, aminopterin, vinca alkaloids, proteinaceous toxins, a fragment of Pseudomonas exotoxin-A, statins, ricin A, gelonin, saporin, interleukin-2, interleukin-12, viral proteins such as E4, f4, apoptin, NS1, and non-viral proteins, HAMLET, TRAIL, and mda-7.

8. The secondary functional moiety according to claim 6, wherein the primary functional moiety is a diagnostic compound containing a radionuclide, a PET-imageable agent, a SPECT-imageable agent, MRI-imageable agent, IRDye800CW, DY-800, ALEXA FLUOR 750, ALEXA FLUOR 790, indocyanine green, FITC, BODIPY, BODIPY FL, rhodamines or rhodamine B.

9. The secondary functional moiety of claim 1, wherein the transition metal complex is a platinum (II) complex and the primary functional moiety is an auristatin or auristatin F.

10. A cell targeting conjugate comprising:
a reacted secondary functional moiety according to claim 1, wherein the halide ligand L₁ or L₂ of the secondary functional moiety of formula I has been displaced by a cell binding moiety.

11. The cell targeting conjugate of claim 10, wherein the cell binding moiety is an antibody, a single-chain antibody, an antibody fragment, a monoclonal antibody, an engineered monoclonal antibody, a single-chain monoclonal antibody or monoclonal antibody or fragment thereof that specifically binds to a target cell, a chimeric antibody, a chimeric antibody fragment, a non-traditional protein scaffold, an affibody, anticalin, adnectin, darpin, Bicycle®, or folic acid derivative that specifically bind to the target cells.

12. The cell targeting conjugate of claim 10, wherein the cell binding moiety is an antibody selected from the group consisting of trastuzumab, cetuximab, rituximab, ofatumumab, obinutuzumab, brentuximab, anti-EGFRvIII antibody, and antibodies directed against intracellular targets of aberrant cells such as tumor cells such as anti-MAGE-HLA peptide complex antibody.

13. The cell targeting conjugate of claim 10, which is selected from the group consisting of:
trastuzumab-Pt((1R,2R)-cyclohexane-1,2-diamine)-auristatin F,
trastuzumab-Pt((1S,2S)-cyclohexane-1,2-diamine)-auristatin F,
trastuzumab-Pt((1R,2S)-cyclohexane-1,2-diamine)-auristatin F,
trastuzumab-Pt(N¹,N²-dimethylethane-1,2-diamine)-auristatin F,
trastuzumab-Pt(propane-1,3-diamine)-auristatin F,
trastuzumab-Pt(1,3-diaminopropan-2-ol)-auristatin F,
trastuzumab-Pt((1R,2R)-cyclobutane-1,2-diyl)dimethanamine)-auristatin F,
trastuzumab-Pt((3R,4R,5S,6R)-3,4-diamino-6-(hydroxymethyl)tetrahydro-2H-pyran-2,5-diol)-auristatin F,
trastuzumab-Pt((4aR,6R,7R,8R,8aS)-6-methoxy-2-phenylhexahydropyrano[3,2-d][1,3]dioxine-7,8-diamine)-auristatin F,
trastuzumab-Pt(2-((2-aminoethyl)amino)ethan-1-ol)-auristatin F, and
trastuzumab-Pt(2,2′-(ethane-1,2-diylbis(azanediyl))bis(ethan-1-ol))-auristatin F.

14. The cell targeting conjugate of claim 10, which is selected from the group consisting of:
anti-EGFRvIII antibody-Pt(1,3-diaminopropan-2-ol)-PNU-159682,
anti-MAGE-HLA peptide complex antibody-Pt(1,3-diaminopropan-2-ol)-alfa-amanitin,
MAGE-HLA peptide complex antibody-Pt(1,3-diaminopropan-2-ol)-PBD, and
brentuximab-Pt(1,3-diaminopropan-2-ol)-alfa-amanitin.

15. The cell targeting conjugate of claim 10, wherein the transition metal complex is a platinum (II) complex, the cell binding moiety is trastuzumab and the primary functional moiety is an auristatin or auristatin F.

16. A method of treating a mammalian subject for cancer, the method comprising:
utilizing the cell targeting conjugate to treat the subject.

17. The method according to claim 16, wherein the cancer is selected from the group consisting of colorectal cancer, breast cancer, pancreatic cancer, and non-small cell lung carcinomas.

18. The method according to claim 17, wherein the cancer is breast cancer having a low expression level of Her2.

19. A pharmaceutical composition comprising:
the cell targeting conjugate of claim 10, and
a pharmaceutically acceptable carrier.
